SEQUENCE LISTING (133 sequences)

SEQ ID NO: 1 - DNA, 24 bp, Artificial
  Designed oligonucleotide primer to amplify a portion of hprt sequence from CHO-K1
tctgcaggcttcctcctcacaccg

SEQ ID NO: 2 - DNA, 25 bp, Artificial
  Designed oligonucleotide primer to amplify a portion of hprt sequence from CHO-K1
tggaaatggcgttgccttgacatgt

SEQ ID NO: 3 - DNA, 25 bp, Artificial
  Designed oligonucleotide primer to amplify a portion of hprt sequence from CHO-K1
tggaaatggcgttgccttgacatgt

SEQ ID NO: 4 - DNA, 20 bp, Artificial
  Designed oligonucleotide primer to amplify a portion of hprt sequence from CHO-K1
caccttttccaaatcctcga

SEQ ID NO: 5 - DNA, 28 bp, Artificial
  Designed oligonucleotide primer to amplify a portion of hprt sequence from CHO-K1
agcttatgctctgatttgaaatcagctg

SEQ ID NO: 6 - DNA, 26 bp, Artificial
  Designed oligonucleotide primer to amplify a portion of hprt sequence from CHO-K1
cttcagtctgataaaatctacagtca

SEQ ID NO: 7 - DNA, 24 bp, Artificial
  Designed oligonucleotide primer to amplify a portion of hprt sequence from CHO-K1
aagacttgcccgagatgtcatgaa

SEQ ID NO: 8 - DNA, 24 bp, Artificial
  Designed oligonucleotide primer to amplify a portion of hprt sequence from CHO-K1
ccaagtgagtgattgaaagcacag

SEQ ID NO: 9 - DNA, 24 bp, Artificial
  Designed oligonucleotide primer to amplify a portion of hprt sequence from CHO-K1
tgtgtgtattcaagaatatgcatg

SEQ ID NO: 10 - DNA, 26 bp, Artificial
  Designed oligonucleotide primer to amplify a portion of hprt sequence from CHO-K1
gctgagaaaatttaacagtattttag

SEQ ID NO: 11 - DNA, 26 bp, Artificial
  Designed oligonucleotide primer to amplify a portion of hprt sequ ence from CHO-K1
caaatacaagcaagaatttcccagag

SEQ ID NO: 12 - DNA, 20 bp, Artificial
  Designed oligonucleotide primer to amplify a portion of hprt sequence from CHO-K1
ggacttgaacatctagggag

SEQ ID NO: 13 - DNA, 24 bp, Artificial
  Designed oligonucleotide primer to amplify a portion of hprt sequence from CHO-K1
acttaccacttaccattaaatacc

SEQ ID NO: 14 - DNA, 26 bp, Artificial
  Designed oligonucleotide primer to amplify a portion of hprt sequence from CHO-K1
gacaatctatcgaaggctcatagtgc

SEQ ID NO: 15 - DNA, 9638 bp, Cricetulus griseus
  Intron (1)..(1): CHO-K1 hypoxanthine phosphoribosyl transferase intron 1
gtgagccccgggactcttcagcagagcgggacagccaggcctaggcggctggctgctcagtggcgccgctcggggcccgttggcgggccttgggtgttggggcccgcggaagtccggggcgggggcggtcaccgcttcccggaatccggctgcgcgtccccagagggcggcgggcagcaggatggaatggcggggttcagggctgggtctcccctgggagccccgagacggttgactgcttgtgaggcaggcaggtggaaatggcgttgccttgacatgtgcggccaccgggagcccggctgggagaggcggagggcgacttggagaaagatacctcacggagaggggagaggggagagggcgggggagctttctcggggagacccttgctcttgaagttcggggcttggcggccagggagagcttcggggggcgcctctggaaggagctccagtaccctacacgacagcatgggagggcacgttcaggcctgggctttgcctctaattctgcagatgtcataattcgtatttgtggaatgcgcaattgctccattgaatccgcgttgcggccctgctactggaaaagcatgtctgttttcccacctgaggagactagtgaaaaggcggctcattttttttcccacggcttggggttggggttgagatacatttgcccttgatgctgtgaggaattttttttttaaatatggatttagttgttaggctaaaggacatttcccccccagtcagcccctgtattttgtctcttctcccattggctctatttttcgtaacctgacaagctgatcccccactctgctccacaaccttgcttgctaaaataacttaaagctcctgcctccgattaaatattttagcctgctttattgtgattattgcccctactactaattaaatgtcatatcattctttattaatatcactgtggtagatttcgtagttacctggttacttgattgtctctcccacttacacaaactaattattttcatttttctggaagggtcttactgtgtagctctgggtggttaggaattggcaatgtaaaccacccggctggtcatgaacttgtgacagtcctcttgactcttgcctcccaagtactgcaacgccatggcctgctgagttttttttttttcgtggtaggaactgaacccagagctttgttcccgataggtgagctgtctacctctgagccaaaggctaccgcccttaaacaagctttataggtcaggaatcaacattgtagctttagcatctagcacaatgcctggtttgtggggagcttccattaatagtatgttgagtgaattaataagcatttgagcctaaaaatgctgtctcatagtcatattagacagtgtggcttaaggtagaaactctgttttttttgtttgtttgtttttttggattttcgagacagggtttctctgtggctttggaggctgtcctggaactagctcttgtagtagaccaggctggtctcagaactcacagagatccacctgcctctgcctcccaagtgctgggattaaaggcctgtgtcaccaacgcccgttgaaacactctgttcttaatgacctggagtcctgctcttgattccccaagtaaaccaggccttggatttacagtcatcttgcttctacctcagtgaggggattacatgcttgcatgtgccatcttctggtttgaggagctggctgctggtgttgaaaggactgtaagacaatatgcaaataaacaagcatttcacagttcctgggtggaaacaggaataataaccatcattgtctttacatagatgatcctaattagttttcagaataaccctgtaacaatggtgtgttcacattatcagacataatagggggagagacccccctcaagtattcagccctggtttagattagcaccagaagcaaggataaattttaggcggtggagctctgccttaaaaaatagatctatctctggagcctatgggcaacagtctgctgttatttttatttggttactgaggagttctacagatgcctttaagttttgagatttggtacttgtagagtatagagctaattagttcttaatcttagaagtgactattcttagttttgtttgaacatcaagaaattcactatttttagcttgcaaattcctgttccttgtttttaagtttccttcccctaaattgtctgcatgttaggaataacaaacacaaaaacacttaactagtgcttactgtgtgctagttgcttttctaagtcctttaactgacttgatttttttcctaatttttctgtatatggctgttttgcctgcatgtatgtatgtaaaccacatgcttgccctgtgcctgtggagtccagaagagctcatcagatcctcaagaactggtatttcagatgattgtgaactgccaatggaggttctggaaatgaacccaggtcctctggaagagtaataagtgctcttattactgagccatcctccaacccctacctgacttactctttaaaaaaatttacacagaggagttaaggaactttgctaaatcattcagtgccattacatttgaacctgttctctggccccagattctacccttaaccagtaaatttaagctaggagctatattaaatgtttcttacatacctttctctgtcttaggcattgacaaatgtttttgcagacttcatctaatgtcaataccctaaagttacataatatggaccctgttgtgtagactaatatgattggtgacacaggtagaccaagtgaccatgcactagatctccttcatgaaactggcccctaggtgttcctgatagatataaaacacgctgcttgtaactatcatttatcaaaaagcttctggctttttgcaaactaatgtgaaacatcaggtatgtttcatatagcatatcgttgaatttcaaaatggcccagatagaagtgtgatttactaactgaacttttacctttctagatgatttttgttgttgtcttttttctgagacatggtttctctgtggctttggaggctgttctggaactaactcttgtagaccaggctggtcttgaactcacagagaaccagctgcctctgcctcccgagtgctgggactacaaccacaggacactagatgatttattttttattttttatttttttaaagtggcctgaaattgactatgtagcctaggccttaaactaacacattcctcctgctttaatattctaagttttaggattacaatcatgatccaccacacttggcttcagggtttggttttatttatttgtttttgtttatttggtttttcggagacagcatttatctgtgtaacagccctggctgtcctggaactagctctgtagatcaggctggccttgaactcacagagatatgcctggagtgctgggattaaaggcatgcaccaccacctggccctaaaggtattttgagatagggactttagttataggctggtctcccaattctcagtcatcctaccttaccctcctgagtgtgagattagaggtaaacatattccaccatgcctagtatgctactcagagagtgagggaaggggagggagagagggagtgagtttttgtgatctaactcaggatcttacatattaagcatatgctttattaccattgggctatacctctatccatctgatacttttttcgtttgttaagatttatttctattttgtatatatgagtgttttccctgtatgtatgtatgtattttttaaagattttatttattatgtatacattctacttcgatgtatatctgcacaccagaagagggcaccagatcttataatggatggttgtgagccaccatgtggttgctggtaattgaactcaggacctctggaagagcagctggtgctcttaacctctgagccatctctccagccctgtatgtatgtatgtgcatgattagcatgcctgtggaggtcagaagagagcacttgatctcctagaactggagttacagacaattgtgagcttccaggtgagtgctgggaaccgaatccatgtcctctgcaaagagcaacaattacacttaaacactgagccatttccagcctgttccctccctcagaattggttaaaatattatatatttttgcttgtcacttttattattatcaacagtgtttaaaataacactcatttaagtaagatgaaaattaactatttatgatcaccaaaaaatggaaacagttcctcttagcgatttctttctgtaaattttgtactgtgttacctatacatccatcttaattattttatgttccagctactgtagcaggtaggaaaagaactaatcatatcagaaaaatgtgccttatagttttattattgttgtttggggtggatggatttctttattagagggttggtttattttagtttgaggcaggttttctttatataggccaggctgagtctctttaaatgttgggactataggaatgcgctgccatgcccagctcttgaggtttctagtttattttgttttcaagtcagggtttctttatgtagccctggctgtcctggaactcactctgtagaccaggctggccttgaactcatagagatcctcctgccttgctgtgctgtgctggggttaaaggttggtgggtgtgtgtgtgtgtgtgtgtgtgtgtgtgtgtgtgtgtgtgtgtgtgtgtgtgtgtgtgttttgtgtgtctgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgagagagagagaggctggccttgaactcatagagatcctcgtgcgtgcgtgcgtgcgcgtgtgctgcctctctgtgtgtgtgtgtgtgtgtgtgtgccactactgcccagccattttattttattaattttatttatcatgtatacagtcttctgcctgcatgccagcagagggcaccagatcttattcatggtagttgtgagtcaccatgtggttgccgggaattgaactcaggacctctggaagagcagtcggtgcacttaaccactgagccatttctccagcccagccatcttatttttttactttaaactcttttagattaagggggaaaaacagctaatatgttttattattacaataactgactgtcatgaactgagttttcattttctttatttttatttttgtgcagagttggtaacagatacctttacaaggttttttaaatttcacccattttttttttaaaattcacttttattttttttgttttgttttatgagacaggctctctacatagccctggatgtcctggaactatgcagatcagcagccggcgttgaattcacagagatttgcctgcctttgcttctagtgctgggattaaagatgtatcatttgtcactacatttggcaaatgtttaaaaaaattaaattttaaatagaattatatccctttttcccttttcttcctccagcccttcccatttacgctccgtaaaatccctccagtgttcaccctctcaagttgatagcctctttttctttatcattgttaaacgcatatattcttgtttgtgtgtaaatggtttcaaggtttattacgtggcagtgaacatccggtaaggggctctttgtggaagcagccattcttcacccagcagttattagttacctgtactttgtctaggggtgggacccagagaaatttccaaaacttggaatcctcctgccttagttttctgagtgctgggattataggcatgcactgtcctgcctagctctagtagtttttacttatttttagtttgatatttgagacagcatctcattatacagccctggctgtccaggaactctgtgtaaaccaggctagcctcaggcctcaacagaaatttatcttcctctgccttctaagtgctgggattaaaggtatgcaccaatgcaccaccacaaacaccaagctttaaataatttttttaagagatgggatctggctgtgttgcccaggttacccctgccttaatcttctacgtagttaggatcccagtggaatgttttttattcttccataattagtcaatgatccatgtcatttttttctcccctatactaaaagggtactagaaatgtgttcagtctttctgtaattttaagttatgtggattcactaacattgatagatcacataaaagtgttaattactaccaataacagtaatattgagactcaggtggtattttcctgttggtgactgccaaattactaacatttaaataatctgctaacatgaattcagacacttcaaattgctatgaaaccatcttcaggtttttgttgtatgtttcagtatgaacatacctattattctactatgcacattagtaaaaggaaaaattatggtattcatgtccttacagatatggtagttataggacccatttgcctagagtggttataatatattcctgggatgaacactccactaccttatttatttttcttttagttttagcccttggaagggaaacaaacagtagatgtattatttgtaaactgataaaaaattgtaagataaacacagtatttaacttgttttcacatttaccagtgtctgtcaataaattattataattggtttaatcataaccagagagtataaattatctcaagaggatagcatatcatttactttaatatttttggaaaattttaatatatttgaattaagctagtgtctcactatgtagccaaggctggcgtcaaacttgagtcttctgagtgctgagatgataggtatgtgccaccatgctcagcaaaatcgctattatgtacacatttataataatggaagtcacataaaaattcttgtggtttacacataaaatttcttgttacacagatatattatcaaaatgatataaaaagggaaatgaacctaagctaatatgatctttggaatatcaattcatcatccacaactaggattgtttaggtctctttgtatttaaaaaatagcattgttcatgagtccaaacaagtggaaatgagttctgaaatgaactatgtgtgttgaacatggtagtaaagccctgtaatcacagcacttggaggcaaaggcaggaggatggggtcagtcatcctcagctacataatgagttcaaagccagcctgagctcatgcaactgtttcaaaagagaaaatgattgtgagttaagacatgtataatactaatacaggtgtagtgaaacatacttgtaatcccagtgcttggaaggcaaaggcatgaagattgccaagagttctaacccagcctaaactacattagtggtgagacaggaacaagaggaggtgagaataatagataaacagaaaaatgtagttttataactaataatcaacatgtcccaacttctataatggaaagaaattgttaggtgtggcctccacatatgtgcacctgctttcacacatgcatacacccacatgaatacaaacacaaatatctcaggtgataggagacataagacttgatttttgccttttggggaagaattgtatcaatatggtcttaataaactgactatgtggcccatacctgtgaaccccttcatttgggaagttgagggagagtgccctaggtgtgcagatactttgggctgtgttgtgagattcagattaccctggactacagagctgagactgaagtcatcaaaaacaagatttttactcttaaaataactataaatattatcaaaagcacaaaataaaaatcctctgacataaatctgttttaattagaaattaaaagttttagattagaaaccagttttgtgtgttgacagaaatagtattcctattttgaagtacttcctggtgttctgtttggattctttgaatacatgactgggagtagggtgagttggtgtatacctgttatctcagcactcaagaagcagaggcaggaggatcatcgcaagcttgaggcagcctgatctatatatctagttccaggacagctggagttacatagaagaccctgtctcaaacacataaaaagactgggatgaacttggcagaattcacattgaatttgtctttgaaacaaacaatcctttaagttcttaagttaccagtacaacctgtaaccaggcacagggccatttaagtaggtgcagtagtttgacaaattctgggtatgatgtggtttaaacttatggcagaggcagtgggtgggtgaaggggatgattaacctcaaggaagtcattgtggtattctaatcttgattctaaagtactttgaaacaatccaacttagtttttgtcagattttagtgcacacgttctgatgagtctggtaggtatgtttatattataatattgagaagtctgtcatcctgtctcaaaacatgaactaaattttgctttataaaacttataaaaatgcgttcataggtgggcaatggtggcacacacctttaatcccagcacttgggaggcagaggcaggtggatctctgagtttgagaccactgtggtctacagagcaagttccagacaggctccaaatctacacagagaaaccctgtctcaaacaaacaaacaaacaaaaaagctttcttgtaaaatactcgttgaatgatcctttttatgatacaaatgtccaagttccatttttgttagtaaaggtattctttttttttcttgagacagggtttctctgtggctttggaggctgtcctggaactagctcatgtagaccaggctggtctcgaagccacagagatccacctgcctctgcctcccaagtgctggaactaaaggcatgcgccaccaatgccaaggtgagtttttaaaggatcaattaggtttttggtgaccccccaccaccacacacacatatatactctgagataagcattcttaggatttggatggttaacaccagttttcattttctgtgtgagattttatttaatttacaagaatatactgcattatattaaatattttaggggattgggaagatgacacagtctataaagtgcatttcatgtcatcttcagaacctgagtttggaacccccagcacctagctaaaaagcaggacattcctataatcccagcaccagggaggcagagacagggggatggatccctggggcttcctgccaaactatactagccaatgagagacctggtctcaaaacataaatgaattttttagacccattttcaatctctggagttgacacacatatgcacttgcatttgcccccacaatgctcaaacatactcaatgtgtagagtagaaactgcgtagactagtctctgatgtttaaagtatttttgccacatttgtcatacctctttaaattgttatctttttctacttgtgacctgaaggaagaggattatagtttattacaaaaatataaaattaactgcccttcataccctctcatacccaaattcctctggcgaactaaggcttattttctaaattttgttaaatatggatatcttatgctctgatttgaaacagctgtctttctttttcag

SEQ ID NO: 16 - DNA, 2539 bp, Cricetulus griseus
  Intron (1)..(2539): CHO-K1 hypoxanthine phosphoribosyl transferase intron 2
gtaagtaagatcttaattgaaggggctggagagatggctcagcggttaagagcactgactgctcttccagaggttctgagttcaattcccagcaaccacatgatggctcacaaccatccctaatgagatctggtgccctcttctggcctgcagggacacatgcagacagaacagatcttaattgaagttttatgggtttattttagatgtgtctgtgtgtatgtttgtgataggcatgtatttgatacatatgtataatgtatatatttatgtaaaagctagagaaggacattgggtgccatactctgttgctctctgtcttgctcccttgagttggtgtctctcactgaacctggggacatgctgattcggttaggctctgtcccctattgctgaagtttcaagcagatgcggccacacctggtttttatatgggtattaaggatctgaatttaggtcctgatgcttgcacaagtgctcccaatggcctgcatgtatgtgcagcatctgcattccaggagaccacaaaagtgtgatgggtcctctggaactggagttgcaggccattgggagctactgtgtggctgggaactggacttagggcctctgcccaaacagcaaatgggcttaactctctccagccccaatattttctagttttaaagttctttttcacaaaaagaaaaacaccaggaagtcctactagttattcagtttgagatataaatagtggtactccataatgagtccagcaaatgttgaaacgaggttggaagtctgtaaatgagattatgattctgttggtttgctatacacatttcttttaaaatttttttataaattttatttaaattagaaacaatcttattttacatatcaatcccagttcactctcactcccatcctcccaagccccccaccaattctcccatccactccccaaggagggcgaggccttccatgagggatcatcaaaaaatctgtcacttggggcagggcctaggccctcctcccctgttatacacatttctttactgtatcactgtatgtaaagtatatatacacatacgagttatgtactatcatataatactaaatttttggactttttaatattagtacatctatagctatcacatgtttttatctttaaaaatttttataattgtgtgtgtgtgtgtgtgtgtgtgtgtgtgtgtgtgtgtgtgtgtgtgtgtatgtatgtgtatgtgtgtgtatgtgtgtaaactgttttctctttgagataggttcttactgtttagttcagtctggcttggaactttctgtatagcccagactgaccttaactcagtattcctcccccatctggagattacaggtatgagccaccatgcccagtcctgtttttatattgattttgaaaaagggtctcaaatagtttagactggcttccaactcacagtgtagctgaggatgacctgaagttctgatgctattgcttctactggtgtgccattatgcccagtttgcacagttatgaggataaaacctaggaccttgttcctgtgcatactaggcatagactctaccaattaagctacattcccagctcccccaggtttactttaaaggtgataatgctattctgttcatggatataccataactgatttaactctgtactgttggtagacacttaagatctccattattttgcacttagcaggagtttattagaggaaatatgatatatattggcacttgctcagctttaggcaagtagaaattctgggtcaagtaaaagatgtttattgaatattatcatactgccaaattaacctctaaaaaatttcataattttatactaccagtggtatgtgagttcatttttcctcacaatctcttctaatattttacattagagaaatgcaatttaaccaaaattcaccagtcttgttagctttgaacattgtatctttttatttgtatttttaaatttcatgatgttatagtatacctgttcttttatttgagacagaatcttattatgtagccctaagtgacctagtacttactctgctgttgtacagtatgtccctcaacccatacccttcctcctgcctcagcctccagagtgctagaattataggcatgtaccatggtgcctagcatgtacctggtttttaaaggtagctggaattataggcatgtaccatgatgcctagcatgtacctggtttttaaagttagtaagttttaattttggttgagctgtgtgttggtgtgtaagtataatctcagcacacagagttcgagacatgggttatgagttactatgcagcctgggctacatagagggatcctgtgtcaccttccccaacccccaaaatcttccctttgccatatggaaaacatcccccactttatttaatagtttgatttatgaagcaagattaccaattatggggacaaagaatgtgtcctgtggaagtttaagaagtgtttgttataaaaatataactatttggaatcttctattcctgattttatttttgtag

SEQ ID NO: 17 - DNA, 6665 bp, Cricetulus griseus
  Intron (1)..(6665): CHO-K1 hypoxanthine phosphoribosyl transferase intron 3
gtaagtataattcattcataatttaaaaaatatggcaatcctagttttgtatgtatttttgtttgtttgtttttactttgaaacagtgtttttctgggtagctttggagcctgtcttggaactctctgctgtagaccatgactgacctacagagatctacctgcctctgactcctgagtgctgagattaaacgagtgcaccaccacctgagcctcatcaactgaggcttatcccagttttttttagaattttagtttgagaccctctctaacttgctcagactatccttgaactcattacataggctgatctagaaattttagtcatcttgactcagtttctcaagtagctgggattacaggcaggtgctaccacttctggccataattgttgctgttattattcactggtactttacatcacaacactgtagttcaagaccaacatcaaagtgaatattgtttgttagtcacccaagatggatttgtacttactttgcatggtttttcttttgtcttattttgacctggattttgacttgttttatgatactgctcaggctagtatcaaactctgtggttcaaataatccttctgtcttagcctcccaaattctgggagtacaagcgtgtatcaccatacctgactgtgttttgatattttccaaaacaacttttaagaattactgcagagtacagtgacagagagagatggttcatcagtcacaagcacttgtagagaagactggggtttagttactggcacccaaaagatggctcacagctatgtagctccagttccaggggacctgatgtcctagcctccagataaaataaaaataagtaagttttacaagaattatgggactggtgaggtggactcagtagtcaaaggcactggctgctctttcagaggacctggttcaattcctagcacccacgtggtagttcacagttgtctttaactccagtcccagggatctgatgccatcttctggcctccaagggcaccagacactaacatggtacacaaacaaatatgcaacttgaacacccatacacatattttaaaaataaacaatttctgttgagacttggtgttttcctgactgtatatgctggtggcaaataatttctctggaactaagttgctttgagagtaatcctaaattatttgaagcaaagtctcattattaaggttaacaattatttctcagaaaacagaaaccacttttgacctttggccacgttacctttgtcagagtctgaagtgatgtagaatattgacaatatgatgaaacaaaatgaatgtaatctttacatgtaaattccatcattcacaattttgattgtgtggcttattttttttattttcattctctctctttttttttttgagactctatgtagctcttactgtcttggaactcactgtatagaccaggctggtcttgaactcagagacccaactgcctctacctcctgagtgctgggattagaggcatgtgccaccactgcctggctaaaatagatatttatttttaaaataaaactgcaggatattatagagaaacttaagtcacccatattctttttgttttttgtttttttttttgtttttggtttttccagacagggtttctctggctttggaggctgtcctggaactagctcttgtagaccaggctggtctcgaactcacagcctgcctctgcctcccgagtgctgggattaaaggcgtgggccaccaacgcctggctctctttttggttttttaagacaggatttctctatgtagctttggagcctatcctggcactcgatctggagaccagactggcctcaaactcacagatatctgcctgctgagtactgggattaaaggcgtgtgccaccaacgcctggctaagtcacccatattctatttttttaagttttatttttattttatatttatgagtgttttgcctacatgcatgtgtgtttaccatatgagtgcctcatgtctacaggtgccagcagaagacatcagatcccctgggactggagtttcagatgtttgtgagtcaacatgtaggtgctaggaactgaacttgggtactctgcaagaatagcacatacatttaactattgaaccatctctccagctcttgtcaccactttttgagtattttaaagtgttttctgtctgtatacatgtacacaatgctactatagtttatataatcagtgttatcccagatgtcatttaactaggtggaaaagaacctgggggttaccaaatatgtatttcattaaatgaaagagtctgctaattaaaacctctgtattttttgtgcttctaagaaagaaaatacctccagttatagtagcatggtaccatgaattatgttttcagaggcagtaaagtatgaatatatatcttaaaacagtgaaatatagtgttatatttggtgacattttttggtcccctatgctctataaccttcaggaaaatgaaacatttttacaagtatacaaatgtgtttcttctattttgagttttatttcaattttataaaattgcactaatagttgatgaaaataatgaggttttgggagaaggggtgttagcaaaccattattttaagttacatttttagatttgtaattaatttattttcagtgttgaatgaatccaagacttcaagtatgctaagcaattacgttgctacttagggattttcaaagatcttagggatggaaaattaggaataaacaggtagacaacgtagaaataaatgttaaaggctgtattttagacccttgagattctctgggaattaagagatttttatgtgtatgagtgttttgcctgcctgaagtctacagaggtcagaagagtgtctgactttctagaattggaattgcatacaattgagacctgccatgggtgtgcttggaattgaacctgggtggtttggaagccaatgctctgaaccacctctgggaatttttggttactgacagtgacacctaaatgaaggaacccttgggcactctgcttaatgaaatgatgaaaagggaaagaagagatggagccaatctggagagaactgtcctttcattcgttccagtcatgtctccatagagtttattctaatagttaaaggtgaggcctctctgagagaggcactaacttaaggatacagtcagttccagatacagggaggttgctgtggtttttctgcccactcaagctaggtaattgattcatggatagcatttgtaaacaatataggcatttatatataacaactattaaatgaacattttattagccccattcaaaaggaggctgttttccttggaaaaagtttcaataaaacataagctgaaacagtgaggtcagtgggactgtggaaatttgaacagcctttttttggtttttcgagacagggtttctctgtagctttggaggctgtcctggaactagctcttgtagaacaggctggtcttgaactcacagagatccacctgcctctgcctcccgagtgctggcattaaaggcgtgcgccaccacagcccggccttgaacagtcttaagagtgccaatgttctgtgtgctttggagttctgttgcctcggaccctatagccatagcctttggatcacagcttcattcttaggtatttactccttttgtcctttccttgaaaagggaattaattggtgttggactgtaataccatgtttctacacaagatgtcagtatttacctaggtttgttattactatgtagcactttagctgtaaactacagctgaaaacctataaactagtttcaggttttctacaattatttttctttttgtcattaattagttctgtgtgtttgagattctgggttttagattagagtaataatatgcaaaattacattaaaaagccaaacatttagtttacagtatgtactagtagtatgcttttatttattttgttttgtttctttcttcccacttccccttacacataccagacaaacattctaccaattgaattatcatttgttttgttttgttgaagtaagagtttctgtgtgtaatcttggctgtcctggaactggtgctatagaccaggttggcctagaactcacagagatccacctgcctctgattccccagggctgggattaaaggcatgagccaccaccacccagctatcttttttaatgatattttgagataggatcttgttaggtagcccagagtgatctcaaactctcaaccctgccttagcctccatcctggaacacgctctgtatgccatgctaggcttaaactcacagaggtctgctgtcctctgcctcccaagagtgttgagactaaagatgtgcactaccaccaccaccacctggcccaacatacttttgttgtgttttgtttttcaagacagggtttctctgagtgacagcccttgccatcctggatccacctttttctgcctccctagtgctgggattaatggcttgtaccaccaccatctagcctaacatacttttaaatacagaacaaaaatagttttataggtagcatatttgtctgctacttggtattattagttaatattgatccatataagtatacattaattttttgtgattctttttaatagttccatagtattattgttgtgtacattaacttgatttatttagttaatcccctgttaccacatgttgcctctttttgatgaaaaaagaatacagtgggctgggcgttggtggtgcacgcctttaatcccagcactagggaggcagaggccagcctggtctccagaatgagtgccaggataggctccaaagctacacatagaaaccgtgtctcgaaaaaccaaaaaaaaaaaaaaaatacagtgggtactttatgttttttgcatgtttctgaatgtttttgtgacattgattcttggaaatggggttgttagaccaaaatggtgtgcatgtttccagttttagtacacattgctgtattcttttcaaaagaggtcatattggtttctgtactccaaccaacaacttgagtatttgtttcccttcaatgtattactaggatttagttctgaaatgtggatgggtgaacactggaatcacatttttttagtagtgcctttttttgttttgtttttcaagacagcatctctctgtgtaaaagagctctggctgtcctagaacttgctctctgtaaaccagactggcctgaactcgtatagatccacctgcctctgcctttcgagtgctgggattaaaagctgagctgccaccattaggctttagtagttctattttaaaaagatatatttatcttactttatgtacatgtgtgaatgccagtatgcataccatgtgcatgctcgatgcttttgaggctagaagggggattagaacacttgaaactggagttacaagcaattataagctgccatgtggatgctgggaactgaacccaggtccattgcaagatcagcatgtgttcttaactgatgaactatctctccagtcccatctagtgggttcttattttgaagtataacatgcattaagaatattggttatagatattttcagttggggttcatgacctgttttttgccctttctagtaatgcaaaaaaaaaaaaaacaaaaacaaaaacaaaaagtatcttactacatttacttccaattcacttatagttttattttttgtatttagattttaatccatctggaatttatttttgtgcatggtgtgaggtagggaccatgctttatattttttctcaaatggataagtaattgaccagatgccatttgttgaataattcatcttttaattactgactcaagatgccatctttgtcatatactaaattgccatatgtttttgggtctgtttctggattttactttgttcctttaactttttgtcaatcactgtactgattgacacaagggaattactgtagctttatggtacttgtcattttcctagtagagaaagtccaggctttttatttatctgctttcaattcagttttctgtcttcatttcagcacatccaaataacagttattcccaccaactacagtgagtgttaagtgctctcttttgaaacccctataagggtttctgctaatttctatgagagcagagtgaaaacctaagaagtgtgttctaacagactggtactcagacctggtttctcagttctgaagatgacttgggtatgggctggactttgagggggcatatcctcagggttggatgtgatacataccagctccttcaacttgtgactcacaggaatgttacagggccagaagcagacacctgccatgctaataaattgtcctttgtggttgagaactgcctgattgatgatgagcagctatattatgctcactcagaaatggatgaactctatttttaggactcttcagaacattaaaaaccagcaatggccagttatgatttttaaattttactaatctctatttacctagtcattaatttctagaaacctaatacttataatacatgatttagaataccataatgttgtagccattagatagctaacttcttacatctaataattttttgtgtgtattcaagaatatgcatgtaaatgatgtttttttttaactag

SEQ ID NO: 18 - DNA, 4473 bp, Cricetulus griseus
  Intron (1)..(4473): CHO-K1 hypoxanthine phosphoribosyl transferase intron 4
gtaagtaccttaaagggaaagcaaagaatcatttttataccaagaatcaattagtaactgtgctttcaatcactcacttggagatactttaagtaagcaattattgtttaatacttgcaaagtacccttatatagttttatatatcttctggactatatagccaaatgacagcagaatctaatagtaacactaaaaatccacattgttaattactgcgttctgtaatacctctttgaaaatctaatttggtatccttcagaaagtacaaatggggagaaaggcagtcttttaaaaagttttatgccgggaggtggtggcacatgcctttaatcccagcactctgcccaggcagaagcagcctggtctacagagtaagttctaggacagccagggctacacagagaaaccctgtctcaaaaaaaaagttggggggggagtttcacatgtagttttatttttttaaagattttatttatttattatgtatacaacattctgcttccatgtatatctgcacaccagaagagggcaccagatctcttaacggatggttgtgagccaccatgtggttgctgggaattgaatcaggacccttggaagagcagtcggtgctcttaacctctgagccatctctccagccccgtagtttcatttttaaggtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtctgtgtgtctgtgtgtctgtgtgtatgtatgtgtatgacgtgcatgagtttgttgagttcaggagccagttctttccagcatgtagaattgagagttgggagtatgttaacatgatcaaaattggcaccaagtacctttactccctcagctatctcactttgtagcccatatcatcccaacatctcctcctgcctcagcctcccaagtgctgggattgcaagcatgagccactatactggactgaatatgttatactaagctgctagttatttacttcagatactgtgccttgtataaatgtattactttggtaaatttcacagtaatctggtaagggaagttatattctcattctgcagatcaggaaattaagcaatatagttacagtttggaaaaatacatgggattatgagacaactgtctcacaatctcaaagtcacacagccaggaagtagaagagcttgaatcctgacatcagagttctttcttggtctagtttttacattttagtgctcacagtttacttcgttgaaaatctgtaggggaactttaacataaaacacttgtgaaattgtatagtcctgtatgcggtcatggcaaactaatgttgtcatcagcggtgtttggaaacttcacacaacttgtaatctgttggttcattctctctcctgtccatttggtatacaactgccagacctccttccctttcattgtgccctgtccatttttagaagccattcaaaacctgcacttgtataatcagtttttagtgctagaaggggctttgccctcagtgccttctgtgatttagtttgatcttgtctttttcacctaatatttctgtcccactacacagctctgcgatcttaaacagatctctgcttctaaagtaactttatgacagttgcagtagattctgtttattgtggataaaatcaaggaataaaaattaagcggagttaaatcttagtggtgagattatgggtttattgaaacatttttcttttgtggaaatttatcttatgtgtgtacacatacagacatacacaaatattttcccattaatacttgagaatgtaggctttgtatctaaaatctacttttattcaaattattcctcttgtacttgtctttgttgattacttaagtcatttgactccttagccttattgttatcattattaaatagacatgtagcaacttgaaataaaactctcagtggtagcaattcttagttttggtaatggagcaatagcctagtacagttttgtattcattggacaaattttaatctttctggactttgttgatttatataagctgtcttatgctaaaatacatagttgtggtccagcaagatgtcttgatgcaggaagacacttgccactaagcctgatgacctgagttcaatacctctgacctccatattcaagctgtgttacaacctcccaaatgaaacataaagtaaatgtaaagaattaaaagatatacttatgactcaagcaaggtcattagaattcagtagttctgcctgtgtcctagaagcccatgttgtttcctagtctataattctgtaatgctacagcagcagtcactgccgctgtgaaagtgctttttgatgggactgtttgctatccttgaaatgttcaaggtagtggtgagaaaatggtgagaaaactgcaaagtaggtagggaaacaattactgactcatgagttaactatatattcagtttggccaaagatttgcatagtagtgtcatgcaatcttcattcttgccctgtaagggaatcatcatcctctttctatagagaaaggtattgaaatttagtcattatcaggtgacagaaccacaattggaataaataaatgccttagtccaatgtttcttacactgtgttaagatttatttcaatagctccattgctttttttctcagtatatgaatgtattttcagtttacttagaatttatatagacctcataataatatcacagttatgttattcatttgctctgtaacctgtacctttagattaccccaattttttatatttgttttatgttctatgttatcctgagctttaaagtgaccttttcttgtccatagaatgggctgcagttgtgagttgctttccagaagttctgggctaaccttgcttgaatattctattcttagacccatgcaggactgtcagatgctgccttctatataataaagtgctttgagacatgggcttattagtttttcttggttacagtttacttttaatgcataagctgtgattctccaatctttgattttcatgcccagtcactaaacagttttttaaggatttacccatagcatatgcatttgtaagttacgtatgaattttgctagtaatttatgttgtaaagtacttaaaactgtaggttaaactagaggaaatatgtcttcagttatttttctattgctttgaagacacaccgtgacccagacaacttataaaaaagaaagcatttaattcagagcttgtttacagtttcagagggtgagtctaggaccatcctggaggggaaccaggaggcagactggcagcatggcattggagcagtggccgagaacatatgttttatctgcaaattgggtgcagaaagtactagagactggggctgatgtggacttctgagacctcagggcctactcccagtgacaaacctcctccaacaaggccacattgactaatccttcccaaagcagttccaccagctagggaccaaacattcaaatgtaagagcctctgggggagggacattctcattcaaaccaccacaagatgaaaaagttgaatttcatgaaacccagtggatgctatatgtactctaccttgataactactaacttagaataatatagagggtgttataactctgatgttctggttgtttggcagagtgctttgagaactatataaacagtatagaataaatcttctgtgcagtgagtattgtgaacatgtaaaacgctgctttgtttctttaactagcataaacacaggaaggttgagtgaggattccggcatggtaaatcagaactataatcttagcatttggtagattgagacaggaatattgctgctagtttgaggccagctttggctatataatgagttctaggccagcctgggctacagaataaaaagagaatagattggaatgggtttaagggtttgtcgtaaaaggaaggattagtatttttccttagtgtagtcccccagatatgtgtacaaccattacactagctttagttgatgggaaaataaaattttaattgcatctctgttatgttaaatacatgatttttggaggcatttgtacagattacacttagaatattagagtcaagatacatgaaaggccagctggttcaggtttctcagtttacatatgggacacagatccagagaaatttcacatgttgcttaagatcttgcattcaggattaaaagcttataaaaaatgaacatatgggtcaaatattctttctaatagtatacttgtttcaagcaggatacagtataatcaaatacaagcaagaatttcccagagttatatgattttactgataattggaaatatatctcacttcattttctgattttcatttctcttttttcttctag

SEQ ID NO: 19 - DNA, 5683 bp, Cricetulus griseus
  Intron (1)..(1): CHO-K1 hypoxanthine phosphoribosyl transferase intron 5
gtaagtgctgcatttatttctaaaatactgttaaattttctagcttagtgtatactataggtaagccagggaaaaattctttctgttttaattggggggggggtgttttctaaagactaagagaagccatgtacagtaaggcatgcctggaatcccaaacactcaggaagctcttgaattccatttaggttagcctgggttacataatagagttcggaaactagagagttcttgtttttaaaataaaacaaaataaaaaaaacacaaacagaaaaggataagtgatgtattttccagctcatgaaaaaagtttctcttattgttttgattggtttcttttctttcttggggggatggggggctcgaggcagggtttctttgtggctttggagactgtcctggaactagctcttgtagaccaggctggtctacaagaacagagatctgcctgcctctgcctcccaagtgctgggattaaaggcctatgccaccaatgcccagctggttttttcttttaaaagtagctataactatatagaggggtatgtgtgtgtatgtgatttactgtgctaagttttattttatggaacatcataaggttcttatttcatcttaatatgagtatcaaatattcttagaaaaactatttatagttcatgtgttattttccatttgctattatgtctttgagtactttgtatgtaataccagtaaagtttatccagtgcttactttgccagctactactgtaaatgatgtagaaacaagcacaagaagggtaaacagattcccaaggtcaccagagagtgagtagagtaaagattcaaactatcactgtctgtagcctgttgcttttcaatgtgatccctaaaaaccaaaagcatcatccaagcaactactgactcacaatctttatgatgaaaaccagtgagcttttcttaagcaaactttgagctaagctttaagaaccaatgatttacttagaaactttctcattaaggccttctttgactgtgctacataaaaatcttgtatttccatgtcaacttcctatccctttcagtattttttcacttgccactgtctaacagcatatacatatacctctttaatagtctatggtctccctatttcatatctgtctgccttactgaactctatacacctgtcagtgatcatcctcctaccttgacgctgctcaattcttcagtaaacattttaatatatttttagtgtggcttcaagaacctcattctccattatttttcattttctctatactttccacattttccagaacatcgtttttctatcatttttctgcccctagttgcttactcgattttataatctctttgtttcctttatgcttttttgtaatatttttgtgcatgtgttttgtatgtctgagtgtatatgcatgtgtatgcaagtatgtatgcctttgcataactacagaggccagaagaggatgtgtgtcctttattgtgctgtctattcatttgagacagggtcctctctctgaaccaggggctcattttattagaagccagcaaaccccagcagtcttcctgcttcttgccttctcagacatgggctgcaggtgggaacaagatgcctagttttttgtttgtttttttgatttttcgagacagggtttctctctggctttgaaactagctcttgtagacctggtctcaaggctggtctcaaactcacagagatccacttgcatgtgctgggagtaaaggcatgtgcccaccactgcccatcagatgcctagtttttatgtgagtactgagatccaaactccagtgctcctggttcccattgagcatcctctgcagtccttattttgtttttttttaacaaaagaaattttattttttaagtggggctctaggaatcaaacccagaagatgctaaataaatgttctaccactaggcttcaccctcagttctttgttttgttgtttgtttgagaaaaaggtagctaagctaaatagtgcaggatatattcaactccatccacctgcctctacctcccaagtcaagtgctggcattacaggcatgtaccatcatctacctcccttctgcccttctctttctcattccatctctgctctcctccctctcctctttctctctcttgcctcctttcctctcacccttccttcagcattatcttggtttttgtttttttttaaagatttattttatttttattatgtatacagtgttctgtctgcatgttgacttgcacctcagaagagggaatcagatcccattgcagatggttgtgagccatcatgtggttgctgggaactgaattcgggaactctagaagagtagccagtgctcttaacctctgagccatctctccagcccccctagaaggaacttgttgtgcagctcaagatggcttttttcttctatgagacgaggtgtgtagccccatttggtctgagtctcacagtgtggaccaggatgacctcctctgccttcagaatgctaccattacagttgggtatcaccgtacctggcctccaagctggctttgaacccactgtgtagcttatgctgactttgaaccccttctgccttagcttctcacatgatggaattacaggtgtgcaccaccattgttccattagtatttttttaattttttctttttcccattttagttctttgagaatttggtatattgcattttgattatgttcacctccactccactccccccaaatctgctcccacttccctatttacccaacattgtatcttcttcttttctttaaatccaacaatacagttagtgctgtacattattcctggatgtgtggccttcactagagagtggtcaccccactaggagccacattgttaaagaaaactgactcctctttgcaacacctaccaattgctatttgcaattaaaggtggtggtagtggcacacgcctttaaacccagcactcgggaggcaggggcaggtagatttctgtgagtttgaggtcagcctggtctacagagcgagttccaggacagcctccaaagccacagagaaaccctgtcccaaaaagccaaaagaaattgatccttcactagaggtaggacatgatgccctcttccccctccatgctggaattttgtctggcctggccctaaacaggttttgtaatgctttctcaactgatatgaactcataagtgcaactgcagtactgtgtcagaaaacactgtttccttgaagttatctacgactttgtggctctttttctttaattaaaaaagtttgttgccaggtgatggtggtacacacctttaatcccagcactagggaggcagacacagaggcagaggcaggcagatatctgtgactacagacagagttccaggacagaaaaacccacacagagaaactctatcttgaaaaaaaaaatagtttgtatttgtttgcatgtgtaaacatgccacagtatgcacatggaggccagaagacaactttgaaattgcttcttctaccttttttaaaaagtttaaattagaaacaagtttctttacatgtcaatcctatgctggtttcccagctatcagtctggggtccataagcaaccccttctaccttttgcatggaatttggggattgaatttaggtcactgggcttatatggagaatggctttattcaccaatccatcttggttgcccttaattgttatttttaagagatttatttatttataatgtacaatgttctgcctacatgtctccctgccggccacaagagggcaccagatctcattatagatggtggtgagccatcatgtggttactgggtatgaactcaggatctcaggaagagtagccagtgctcttaacctctgagccatctctccaaccaccttacttttttcagtaatattataaaaacatcttcatctcgctacgcagaaatcagtccccttagcctttatttatttatttatttatttatttgtttgtttattttttggtttttcgagacagggtttctctgtggctttggaggctgtcctagaactagctcttgtagaccaggctggtctcaaactcagagatttgactgcctctgcctctcgagtgctgggtttaaaggtgtacactgccaccacccggctagccttcattttttatttcttccttccccttctctactttctaagatttgcctattgaggacatagcacaaatggaattacatagtaattaattgtactttgtgtctggcttttttcaattattttaagatagggtctcaccatgtggccctggctgattagctatgtaatcgaggctggctttgaaccacacaccccacaaataaataaacattcactcaggatgatagcactgtatagttacagtaattatccaaatgaggcatagttttctctctggctgttggccagggattgctctcatttacagagatcatccctcatgtcctacacagcctctcatagcatgcctgctcacttcttccactctgtactctagggggcggtaatttttgtgccatcagtattctgcctgccacaccctagaatcttggtttttcacaagtctctagccctgaatttctaatgtccacattgtcttacgtatttttttttcttttggttttttgagaaagggtttctctgtgtagcttttggagcctatcctggcactctctggagaccaggctggcctcgaactcatagagaaccgcctgcctctgcctcccgagtgctgggactaaaggcatgcaccaccaacgcctggctttgtcttacggatttttatttgttttctttttcttttctttcttgttgtttgttttttgtttgtttttgtttttgagacaggatttctctgtattattttggaacctgtcctggaactcactctgtataccaggctggcctcgaactcacagagatccacctgcctctgcttcccgagtgctgggattaaaggtgtgcactaccaacgcccgactttttatttgttttctgataccagttttggtaatactggagatctgcggctcagtggatcttatgcaggtcttttacccctgataaagtcattttatatagtgaaacttagccatttgaatgccttcaaaaataatacaaagcaccatttctcacagtattagagtttgagatgaaagagtccctatcttgaagttatggagaaaaatctaaaatacttttacagaattaagcatagtggcttatgtctgccatcctagctgtcaggagtctaaataggaggctcttgagtttaaggacactagcctgagctatttagctagacccggtgtggaaaaacaacaagaagatttacatgtatgtacattgcttacatgtacccatttcaattcattgtttttgacctcactatacctatgcagcctagttctgggaactgacattcccactacttgctgaaagccagatgggttgcaagttctagaatctctgtgtgtgaattgtgagaatttttttatgtgctgaaaaataatgacatcgtactgtggaataccagaacactgggattctgtatattctgtgtttagatagtgactctgattttaaaattatgctggttttacttaccacttaccattaaatacctcttttctttttaaaag

SEQ ID NO: 20 - DNA, 4534 bp, Cricetulus griseus
  Intron (1)..(4534): CHO-K1 hypoxanthine phosphoribosyl transferase intron 6
gtatgtatgccattttaaagtagaatgtttttttttcattgggagcaataagcaattgcttctttgtaaggagaaatgctttcagctctctttttatcttgaaaaggtgatgtaatcttaaaatttagtatactttgttattaaaacccttcattatatagtcaactacttgtctaatacagtatatacagggacttggattcagaaccacctcagacactaagctccctagatgttcaagtcctttgtaaaaaaaaaaaaagtcagatgggctggcaagaggactcagtgggtgaagtcacctgctgccaaccctgatgacctgagttcaaaccctgggacccacatggtagaaggagagaacggactcctagaaatttccctgtgacgtgtgtatcacaacacatgtcccccaacccacagacaagtaaatgtaatttaatttttttaaatgtcaagagtgttttcacagaacctatgcatattctcccttatactttaatacctattgcaatgtcatataaataattatgctgaatcatctaaggaaatatgtatagttttagtatagtttcttagactatttgtgtgtgtgtgtgtgtgtgtgtgtgtgtgtgtgtgtgtacatgcaggtgaatgcacacaggcacatatgcaaaggccagagcaggattggatatcttcctctattgtgtaatcagggtctctaactgaggtataagtttaaagttttagccatgcaggagtttggtgggtgagcttttgaatccctctctaacctcacagtactggagtcacaaacacatacagccatggccagatttttatgtgaatactgaagaggcaaactcaggtacctgtgctttaggaccaacttgatacacaaggtttagatacagaactctcacacatatgtagagtcaagtaacaattggttttgccacttgctatttatatcagagttgtcctgagtagcatgcaaggatggatctaggagcccccttgtgaaacagaaaccggcgatacccatagggttgggctctgaacatgtggatctcctgaagttgagtggtggataaggcatcctgttttgtgtcatcattgacattttacaatacctgctttttttctgaagcagtcacatatcttcctggccgtatctagatgtttggctttgctttcatagttctttctgtttttatccttagttacatagtttgatcctacaacagttgttatcgccatcttacagatgagaatgttgaagcgtagggaattgcccacagacagagctaataagctgtagagagatgatttgaatacagggtacctggctcagttgaggcttctgttattttaagttggggtttttgggttttttgcttttgtttttcgagacagtgtttctctgtggctttatcaggctggtctcgaactcacagagatccgcctgcctctctgcctcctaagtgctgggattaaaggtgtgctacaccaatgcccagtttaagttgtttttttttttattgttagaagtaacattgttttgttgttgttgaattttgctttgtttttactttacaatttcatttttattttcaaatctttgactaatctgtgttttttgtgtagaggaaagttcgggtctgtttttcttttttccctgaatgattaactggttcttgacattttttgaattacacttatttattgtggcagggatgtgggcatgtgccacatgcacttgtgtaggtcagaggatagcctgggggagttaggtctctaacttgttggtcgtaggtgggggtagaatacagcttggtagacttggcagcaatttccttacccactgaaccatctctgctgaccaattagctgccttttataaaccattttaaaggatcaaagtatcttgtaaagtctttgaatatcattttaataaagcctatttgagcagttcatcctcaaggactcttctgatagatggtgcaagtgtacctcagtgagtttttgtcttgctggaggtgctatatgctcaagagaatgggtaatttttgataatgtagttgtcctatacaggtgtgttttgtgtttttcccaaaataaaaattgaaaagctattatttgatatgaatatatgatagtatggccactctagaaaacaatttttttttgaaaactgtacatgtaactaccagaaggccttacaaattatactgggtacttatacctgagaagtaaaagtgcacattgatataaaaatttatataagttgatagctaggtatggtggttcacccgtgacgtcagcatttggaaagtggaggcaggaggtcacattgaatctaagacaagcagcctgagttttcatagctgtctcgtagcttcctactcaaatctgtaccagagtattcacaacaccatttgtgatagcccacaattaaaagcgacaaactgaacaaaactgtagtacacctataccatcatattcatcagtaaacaggaacaactattgattgatatacaacagttcagagaagtctaagtggaactgtaccaattatgactttcagtagtacataaagcaggtgttttgctctgtttaaactgcttacctgccccattttcccccaagactattataagcccccctcaaagaaaagtaataaaactgtatgattctacttaaattacctactttgaaatggcaaagtcatacagatgagaacagattaggggttgccaggggttaagggaatgcagaagggagccttgcatgtttcaaatatcttgattatggtagtgattagataattccacatgtgactaaaattgcctgaaccgcacaaaaagagatgagtttatataaaattggtgaaatctgtacatggtcacttagttcatcaatgcccatgtcctggtttgataatatgctgaaaaaggacactggagacctcccttttgtatgatttttttcatttgcaacttcctgtgaaaataagaaacattgaaagccattaactattttggtggtattagggattcaactcagggttgtatgcatacatgacaagcattcagataattagctttagagctgtatgtaattaattctgtaatcaaattacagttgcaaattcatgatttctataagtactcaataacagcaaagtaattttaaaaaatgaaactgctcaaagctattatgtttttagcaagagtcaaacagttgaaaatataattaactccaaatgatgtgagggacgttgatggtcccccgtggagggcttcactatccatggatgggggtgggttgggggaggtcggcagagagcataggaggatgggagggtgagggaatgggggactgatatgtaaataagagtgcttctaaaattaaaaaaggaaaatataattaactaaaaaatggagtaggtagctcagtgataatatacatcctttttttgctggggggagggggggatgagttcaagatggggtttctctgtggcttttgtggctgtcctgcaactagcttttgtagaccaagctggtctcgaactcacagagatccgcctgcctctgcctcccaagtgctgggattaaaggtgtgtgccaccagtgcccagctggtaaaatccattcttaacatgatcaagtctgtgggttcaatctccacagcaaaaaataatttaaaaaatggttcttttggggctggagagatggctctgcggttaagagcactgactgttcttccagaggtcctgagttcaattcccagcaaccacatagtggctcacaaccacgttgaatgagatctagtgccctctgctggcatgcaggcagaagactgtatacaaaataaataaataaataaataaataaataaataaataaataagtaaataaataagtaagtaaataaataagaattattttaaaaaatggttcttttttttgagacagggtttctctgtagcgttggagcctatcctggcactccctctggagaccaggctggcctcgaactcagagatccgcctgcctctgcctcccgagtgctgggattaaaggcatgcgccaccaacttttaacacacttgttttgtagcctaaagttcccatttgtgaggcagttattaagagtcttgatgaaagagcattttttacccacctcctttataagttgaggggttgtatttcaaggattctgaatctagatcccttatttttgaatctttgtgtgttggtaccttggcccattcattattctcagtagctgcacttcaaaggacatatgggtagttaggattccactctcagaagaagcatcttatcaggttccaggtggagatcagtcaggggccttgtcttgttgtaaactctgatgtgggtctccatttgtcttcctatgggtatgcaaaactgaccttattctaaatgcagatgatactatttaagaacactgatgatgttttcttttataatgttctattgtctttccatatgtcacatgtaatattttgtaattaacag

SEQ ID NO: 21 - DNA, 166 bp, Cricetulus griseus
  Intron (1)..(166): CHO-K1 hypoxanthine phosphoribosyl transferase intron 7
gtaagtagatgccctttgggtgatcattttatctatcatagctgaaaggtgactaaaattgcttagagttattttagagaacgtttgtaaagaaggcacatttgcaaatattagatttgaaagtactgttttctctgtctaaataataaactgtgattctttacag

SEQ ID NO: 22 - DNA, 976 bp, Cricetulus griseus
  Intron (1)..(976): CHO-K1 hypoxanthine phosphoribosyl transferase intron 8
gtaagtaatgtttctttttttctcgctctcatttttcaaaaaacccacataaaaatttaggaaaggaaagaattgttttctccttctagcacctcgtcatttgaccagactgatggttcccattagtcacataaagctgtagtcaagtacagacgtccttagaactggaacttggccaggctagggtgacacttcttgctggctgaaatagttgaacagccttaatttacaataactgcctcattgttatttcacatgataaatatagtcataaataagaaataaataattccatttaaatcttatgattgaccctacttcagatacctgcctcccactctggaggcaagaatccctcaggtgagtttatgaacatggtccaggagaaattaagaagactacgtggacagcattatctaaagcagttttgtttgtttgattggttttcaagatagagtttctctgtttaaccactctggttgtcctggaactcaatttgtagaccagaccacagagatctgcctgcctctgcctctcaagtgctgggattaaaggcccaaagtacagtttttatttcatttttttaatcacttaccctattttctttgagacaggatttatcttgtagtactggctgtcctgaaactcactctgtagaccagattggcctcgaactcacagagaatccatctctctctctattgggattaaaggcttttgccatcatcacctagccacactgatccctattttgatatgccttcaatgcccggctttatatgtttttcaaagttacataataagagaaagaaaaaagtacactaatggaacatatttctgctttgttttcaaaatgtgttctcttcaagttactggtccaaagtaccttgtttggtaggaacccagacaattctctaatgttgctcttacctctccagaatatatttttctaatgtgagtttcttttcttttgcag

SEQ ID NO: 23 - DNA, 40 bp, Artificial
  Designed oligonucleotide primer to amplify a portion of hprt sequ ence from HT1080
cctgcaggtcgcgattggtacttgttcagctttattcaag

SEQ ID NO: 24 - DNA, 41 bp, Artificial
  Designed oligonucleotide primer to amplify a portion of hprt sequ ence from HT1080
gtcgacaaggacgcgttctgataaaatctacagtcatagga

SEQ ID NO: 25 - DNA, 67 bp, Artificial
  Designed oligonucleotide primer to amplify a portion of hprt sequ ence from HT1080
gtcgacctctagctagagcttggcgtaatcatggtctccggagactgaagagctattgtgtgagtat

SEQ ID NO: 26 - DNA, 46 bp, Artificial
  Designed oligonucleotide primer to amplify a portion of hprt sequ ence from HT1080
acatgttctcttaagtcgcgaagtagtgttatgatgtatgggcata

SEQ ID NO: 27 - DNA, 35 bp, Artificial
  Designed oligonucleotide primer to amplify the E.coli origin and ampicillin resistance gene
acatgtgagcaaaaggccagcaaaaggccaggaac

SEQ ID NO: 28 - DNA, 40 bp, Artificial
  Designed oligonucleotide primer to amplify the E.coli origin and ampicillin resistance gene
cctgcagggacgtcaggtggcacttttcggggaaatgtgc

SEQ ID NO: 29 - DNA, 36 bp, Artificial
  Designed oligonucleotide to make a linker
cgcgtatctctagaataatcgatagaaagcttacag

SEQ ID NO: 30 - DNA, 36 bp, Artificial
  Designed oligonucleotide to make a linker
gatcctgtaagctttctatcgattattctagagata

SEQ ID NO: 31 - DNA, 30 bp, Artificial
  Designed oligonucleotide primer to amplify the SV40 promoter, SV40 polyA signal and neomycin resistance gene
cctttctagacttctgaggcggaaagaacc

SEQ ID NO: 32 - DNA, 79 bp, Artificial
  Designed oligonucleotide primer to amplify the SV40 promoter, SV40 polyA signal and neomycin resistance gene
cttatcgattcacacaaaaaaccaacacacagatgtaatgaaaataaagatattttattgtgggcgaagaactccagca

SEQ ID NO: 33 - DNA, 30 bp, Artificial
  Designed oligonucleotide primer to amplify an antibody variable region
taaaaggtgtccaggatgtgcagtttcagg

SEQ ID NO: 34 - DNA, 30 bp, Artificial
  Designed oligonucleotide primer to amplify an antibody variable region
gaggccgatgaaacagtgaccagagtccct

SEQ ID NO: 35 - DNA, 30 bp, Artificial
  Designed oligonucleotide primer to amplify an antibody constant region
catcggcctccaccaagggcccatcggtct

SEQ ID NO: 36 - DNA, 30 bp, Artificial
  Designed oligonucleotide primer to amplify an antibody constant region
ttaagcggccgctcatttacccggagacag

SEQ ID NO: 37 - DNA, 70 bp, Artificial
  Designed oligonucleotide primer to amplify an antibody constant region
ggtcgccaccatggagtttggactgagctgggttttccttgttgctattttaaaaggtgtccaggatgtg

SEQ ID NO: 38 - DNA, 30 bp, Artificial
  Designed oligonucleotide primer to amplify an antibody variable region
gcccttggtggaggccgatgaaacagtgac

SEQ ID NO: 39 - DNA, 30 bp, Artificial
  Designed oligonucleotide primer to amplify an antibody variable region
ttccggtaccggtcgccaccatggagtttg

SEQ ID NO: 40 - DNA, 28 bp, Artificial
  Designed oligonucleotide primer to insert a NotI site into the upstream of CMV promoter
atgcggccgcatgtcaatattggccatt

SEQ ID NO: 41 - DNA, 29 bp, Artificial
  Designed oligonucleotide primer to insert a NotI site into the upstream of CMV promoter
gacatgcggccgcatgggaggagaccggg

SEQ ID NO: 42 - DNA, 30 bp, Artificial
  Designed oligonucleotide primer to amplify a spacer
ttgcggccgcgaaaaagcctgaactcaccg

SEQ ID NO: 43 - DNA, 30 bp, Artificial
  Designed oligonucleotide primer to amplify a spacer
ttgcggccgcgacggtgtcgtccatcacag

SEQ ID NO: 44 - DNA, 60 bp, Artificial
  Designed oligonucleotide primer to amplify IGH
ccttggtaccgaagccgctagcgctaccggtcgccaccatggagtttggactgagctggg

SEQ ID NO: 45 - DNA, 30 bp, Artificial
  Designed oligonucleotide primer to amplify IGH
ccttagatcttcatttacccggagacaggg

SEQ ID NO: 46 - DNA, 30 bp, Artificial
  Designed oligonucleotide primer to amplify IGH
ccttacgcgtgacggtgtcgtccatcacag

SEQ ID NO: 47 - DNA, 30 bp, Artificial
  Designed oligonucleotide primer to amplify IGH
cttacgcgtaacgtctcgccctttggtctc

SEQ ID NO: 48 - DNA, 30 bp, Artificial
  Designed oligonucleotide primer to amplify an antibody variable region
cccaggtgccagatgtgacatcaagatgac

SEQ ID NO: 49 - DNA, 30 bp, Artificial
  Designed oligonucleotide primer to amplify an antibody variable region
gccacagttcgttttatttccaactttgtc

SEQ ID NO: 50 - DNA, 30 bp, Artificial
  Designed oligonucleotide primer to amplify an antibody constant egion
tggaaataaaacgaactgtggctgcaccat

SEQ ID NO: 51 - DNA, 29 bp, Artificial
  Designed oligonucleotide primer to amplify an antibody constant region
ttaagcggccgcctaacactctcccctgt

SEQ ID NO: 52 - DNA, 80 bp, Artificial
  Designed oligonucleotide primer to amplify an antibody constant region with secretion signal
ggtcgccaccatggacatgagggtccccgctcagctcctggggctcctgctgctctggctcccaggtgccagatgtgaca

SEQ ID NO: 53 - DNA, 30 bp, Artificial; identical to SEQ ID NO: 49
  Designed oligonucleotide primer to amplify an antibody variable region

SEQ ID NO: 54 - DNA, 30 bp, Artificial
  Designed oligonucleotide primer to amplify an antibody variable region
ccttggtaccggtcgccaccatggacatga

SEQ ID NO: 55 - DNA, 30 bp, Artificial
  Designed oligonucleotide primer to amplify a spacer
ttgcggccgcctgtgatggacgacaccgtc

SEQ ID NO: 56 - DNA, 30 bp, Artificial
  Designed oligonucleotide primer to amplify a spacer
ttgcggccgcctattcctttgccctcggac

SEQ ID NO: 57 - DNA, 58 bp, Artificial
  Designed oligonucleotide primer to amplify IGL
ccttggtaccgaagccgctagcgctaccggtcgccaccatggacatgagggtccccgc

SEQ ID NO: 58 - DNA, 30 bp, Artificial
  Designed oligonucleotide primer to amplify IGL
ccttagatctctaacactctcccctgttga

SEQ ID NO: 59 - DNA, 30 bp, Artificial
  Designed oligonucleotide primer to amplify IGL
ccttaagcttctattcctttgccctcggac

SEQ ID NO: 60 - DNA, 30 bp, Artificial
  Designed oligonucleotide primer to amplify IGH
cttaagcttaacgtctcgccctttggtctc

SEQ ID NO: 61 - DNA, 38 bp, Artificial
  Designed oligonucleotide primer to amplify a portion of hprt sequence
ttcctgcaggtcgcgaaggagtttattagaggaaatat

SEQ ID NO: 62 - DNA, 30 bp, Artificial
  Designed oligonucleotide primer to amplify a portion of hprt sequence
ttacgcgttgataaaatctacagtcatggg

SEQ ID NO: 63 - DNA, 28 bp, Artificial
  Designed oligonucleotide primer to amplify a portion of hprt sequence
ttggatccgactgaagagctactgtgta

SEQ ID NO: 64 - DNA, 34 bp, Artificial
  Designed oligonucleotide primer to amplify a portion of hprt sequence
ttacatgttcgcgaatcagatccctgggactgga

SEQ ID NO: 65 - DNA, 20 bp, Artificial
  Designed oligonucleotide primer to amplify a portion of hprt sequence
tgttcctgtgcatactaggc

SEQ ID NO: 66 - DNA, 24 bp, Artificial
  Designed oligonucleotide primer to amplify a portion of hprt sequence
ccagagaaattatttgccaccagc

SEQ ID NO: 67 - DNA, 41 bp, Artificial
  Designed oligonucleotide primer to amplify a portion of hprt sequence
ttcctgcaggtcgcgagtctgtgtgtatgtttgtgataggc

SEQ ID NO: 68 - DNA, 39 bp, Artificial
  Designed oligonucleotide primer to amplify a portion of hprt sequence
ttccatggtcgcgatgaaggttatagagcataggggacc

SEQ ID NO: 69 - DNA, 20 bp, Artificial
  Designed oligonucleotide primer to amplify a portion of hprt sequence
gacacatgcagacagaacag

SEQ ID NO: 70 - DNA, 20 bp, Artificial
  Designed oligonucleotide primer to amplify a portion of hprt sequence
gtttgctaacaccccttctc

SEQ ID NO: 71 - DNA, 28 bp, Artificial
  Designed oligonucleotide primer to amplify a portion of hprt sequence from CHO-K1
agaacgcgtccacacaatcagaaccaca

SEQ ID NO: 72 - DNA, 27 bp, Artificial
  Designed oligonucleotide primer to amplify a portion of hprt sequence from CHO-K1
gacgatctagaggtggttttcacaaca

SEQ ID NO: 73 - DNA, 20 bp, Artificial
  Designed oligonucleotide primer to amplify a portion of hprt sequence from CHO-K1
agggactttccattgacgtc

SEQ ID NO: 74 - DNA, 30 bp, Artificial; identical to SEQ ID NO: 47
  Designed oligonucleotide primer to amplify a portion of hprt sequence from CHO-K1

SEQ ID NO: 75 - DNA, 28 bp, Artificial
  Designed oligonucleotide primer to amplify a portion of hprt sequence from CHO-K1
agaaagcttccacacaatcagaaccaca

SEQ ID NO: 76 - DNA, 27 bp, Artificial
  Designed oligonucleotide primer to amplify a portion of hprt sequence from CHO-K1
gacgaattcgcggtggttttcacaaca

SEQ ID NO: 77 - DNA, 26 bp, Artificial
  Designed oligonucleotide primer to amplify a portion of hprt sequence from CHO-K1
tatgaattcgtcgccaccatggacat

SEQ ID NO: 78 - DNA, 30 bp, Artificial
  Designed oligonucleotide primer to amplify a portion of hprt sequence from CHO-K1
tgtaagctttaccacatttgtagaggtttt

SEQ ID NO: 79 - DNA, 28 bp, Artificial
  Designed oligonucleotide primer to amplify a portion of hprt sequence from CHO-K1
tttctagatagttatgagcccatgtccc

SEQ ID NO: 80 - DNA, 28 bp, Artificial
  Designed oligonucleotide primer to amplify a portion of hprt sequence from CHO-K1
tttctagacggtgaaatcctgtctctac

SEQ ID NO: 81 - DNA, 30 bp, Artificial
  Designed oligonucleotide primer to amplify a portion of hprt sequence from CHO-K1
ttggcgcgcctagttatgagcccatgtccc

SEQ ID NO: 82 - DNA, 28 bp, Artificial
  Designed oligonucleotide primer to amplify a portion of hprt sequence from CHO-K1
ttacgcgtcggtgaaatcctgtctctac

SEQ ID NO: 83 - DNA, 28 bp, Artificial
  Designed oligonucleotide primer to amplify a portion of hprt sequence from CHO-K1
ttagatcttagttatgagcccatgtccc

SEQ ID NO: 84 - DNA, 28 bp, Artificial
  Designed oligonucleotide primer to amplify a portion of hprt sequence from CHO-K1
ttagatctcggtgaaatcctgtctctac

SEQ ID NO: 85 - DNA, 38 bp, Artificial
  Designed oligonucleotide primer to amplify a portion of hprt sequence from CHO-K1
ttcctgcaggtcgcgatggaatcttctattcctgattt

SEQ ID NO: 86 - DNA, 34 bp, Artificial
  Designed oligonucleotide primer to amplify a portion of hprt sequence from CHO-K1
ttacatgttcgcgatcagcactcaggagtcagag

SEQ ID NO: 87 - DNA, 36 bp, Artificial
  Designed oligonucleotide primer to amplify a portion of hprt sequence from CHO-K1
ttcctgcaggtcgcgagatgcctagcatgtacctgg

SEQ ID NO: 88 - DNA, 39 bp, Artificial
  Designed oligonucleotide primer to amplify a portion of hprt sequence from CHO-K1
ttacatgttcgcgataagtacaaatccatcttgggtgac

SEQ ID NO: 89 - DNA, 40 bp, Artificial
  Designed oligonucleotide primer to amplify a portion of hprt sequence from CHO-K1
ttatgcattcgcgaatctcaggtgataggagacataagac

SEQ ID NO: 90 - DNA, 28 bp, Artificial
  Designed oligonucleotide primer to amplify a portion of hprt sequence from CHO-K1
tttgatcagactgaagagctactgtgta

SEQ ID NO: 91 - DNA, 36 bp, Artificial
  Designed oligonucleotide primer to amplify a portion of hprt sequence from CHO-K1
tttcatgaatcgcgaatcagcactcaggagtcagag

SEQ ID NO: 92 - DNA, 31 bp, Artificial Sequence
  Designed oligonucleotide primer to amplify a portion of hprt sequence from DG44
tcgcgagtctgtgtgtatgtttgtgataggc

SEQ ID NO: 93 - DNA, 28 bp, Artificial Sequence
  Designed oligonucleotide primer to amplify a portion of hprt sequence from DG44
acgcgttgataaaatctacagtcatggg

SEQ ID NO: 94 - DNA, 26 bp, Artificial Sequence
  Designed oligonucleotide primer to amplify a portion of hprt sequence from DG44
ggatccgactgaagagctactgtgta

SEQ ID NO: 95 - DNA, 31 bp, Artificial Sequence
  Designed oligonucleotide primer to amplify a portion of hprt sequence from DG44
tcgcgatgaaggttatagagcataggggacc

SEQ ID NO: 96 - DNA, 46 bp, Artificial Sequence
  Designed oligonucleotide primer to amplify the E.coli origin and ampicillin resistance gene
tcgcgacctgcaggtccggagcggccgccatgtgagcaaaaggcca

SEQ ID NO: 97 - DNA, 46 bp, Artificial Sequence
  Designed oligonucleotide primer to amplify the E.coli origin and ampicillin resistance gene
tcgcgacctgcaggtccggagcggccgcaggtggcacttttcgggg

SEQ ID NO: 98 - DNA, 36 bp, Artificial Sequence; identical to SEQ ID NO: 29
  Designed oligonucleotide to make a linker

SEQ ID NO: 99 - DNA, 36 bp, Artificial Sequence; identical to SEQ ID NO: 30
  Designed oligonucleotide to make a linker

SEQ ID NO: 100 - DNA, 26 bp, Artificial Sequence
  Designed oligonucleotide primer to amplify the SV40 promoter
tctagacttctgaggcggaaagaacc

SEQ ID NO: 101 - DNA, 26 bp, Artificial Sequence
  Designed oligonucleotide primer to amplify the SV40 promoter
acatgtcgaaacgatcctcatcctgt

SEQ ID NO: 102 - DNA, 22 bp, Artificial Sequence
  Designed oligonucleotide primer to amplify the mouse dhfr gene
ccatggttcgaccattgaactg

SEQ ID NO: 103 - DNA, 78 bp, Artificial Sequence
  Designed oligonucleotide primer to amplify the mouse dhfr gene
atcgattcacacaaaaaaccaacacacagatgtaatgaaaataaagatattttattttagtctttcttctcgtagact

SEQ ID NO: 104 - DNA, 30 bp, Artificial Sequence; identical to SEQ ID NO: 33
  Designed oligonucleotide primer to amplify an antibody variable region

SEQ ID NO: 105 - DNA, 30 bp, Artificial Sequence; identical to SEQ ID NO: 34
  Designed oligonucleotide primer to amplify an antibody variable region

SEQ ID NO: 106 - DNA, 30 bp, Artificial Sequence; identical to SEQ ID NO: 35
  Designed oligonucleotide primer to amplify an antibody constant region

SEQ ID NO: 107 - DNA, 30 bp, Artificial Sequence; identical to SEQ ID NO: 36
  Designed oligonucleotide primer to amplify an antibody constant region

SEQ ID NO: 108 - DNA, 70 bp, Artificial Sequence; identical to SEQ ID NO: 37
  Designed oligonucleotide primer to amplify an antibody with secretion signal

SEQ ID NO: 109 - DNA, 30 bp, Artificial Sequence; identical to SEQ ID NO: 38
  Designed oligonucleotide primer to amplify an antibody

SEQ ID NO: 110 - DNA, 30 bp, Artificial Sequence; identical to SEQ ID NO: 39
  Designed oligonucleotide primer to amplify an antibody

SEQ ID NO: 111 - DNA, 28 bp, Artificial Sequence
  Designed oligonucleotide primer to amplify the CHEF1-alpha promoter
agaacgcgtccacacaatcagaaccaca

SEQ ID NO: 112 - DNA, 27 bp, Artificial Sequence
  Designed oligonucleotide primer to amplify the CHEF1-alpha promoter
gacgatctagaggtggttttcacaaca

SEQ ID NO: 113 - DNA, 30 bp, Artificial Sequence; identical to SEQ ID NO: 48
  Designed oligonucleotide primer to amplify an antibody variable region

SEQ ID NO: 114 - DNA, 30 bp, Artificial Sequence; identical to SEQ ID NO: 49
  Designed oligonucleotide primer to amplify an antibody variable region

SEQ ID NO: 115 - DNA, 30 bp, Artificial Sequence; identical to SEQ ID NO: 50
  Designed oligonucleotide primer to amplify an antibody constant region

SEQ ID NO: 116 - DNA, 29 bp, Artificial Sequence
  Designed oligonucleotide primer to amplify an antibody constant region
ttaagcggccgcctaacactctcccctgt

SEQ ID NO: 117 - DNA, 80 bp, Artificial Sequence; identical to SEQ ID NO: 52
  Designed oligonucleotide primer to amplify an antibody with secretion signal

SEQ ID NO: 118 - DNA, 30 bp, Artificial Sequence; identical to SEQ ID NO: 49
  Designed oligonucleotide primer to amplify an antibody

SEQ ID NO: 119 - DNA, 30 bp, Artificial Sequence; identical to SEQ ID NO: 54
  Designed oligonucleotide primer to amplify an antibody

SEQ ID NO: 120 - DNA, 20 bp, Artificial Sequence
  Designed oligonucleotide primer to amplify a portion of hprt sequence
gacacatgcagacagaacag

SEQ ID NO: 121 - DNA, 20 bp, Artificial Sequence
  Designed oligonucleotide primer to amplify a portion of hprt sequence
gtttgctaacaccccttctc

SEQ ID NO: 122 - DNA, 20 bp, Artificial Sequence
  Designed oligonucleotide primer to amplify a portion of mouse-dhfr sequence
ctcaggaatggagaaccagg

SEQ ID NO: 123 - DNA, 26 bp, Artificial Sequence
  Designed oligonucleotide primer to amplify a portion of mouse-dhfr sequence
ttaaccatggttcgaccattgaactg

SEQ ID NO: 124 - DNA, 20 bp, Artificial Sequence
  Designed oligonucleotide primer to amplify a portion of hprt sequence
tgttcctgtgcatactaggc

SEQ ID NO: 125 - DNA, 24 bp, Artificial Sequence
  Designed oligonucleotide primer to amplify a portion of hprt sequence
ccagagaaattatttgccaccagc

SEQ ID NO: 126 - DNA, 9638 bp, Cricetulus griseus; identical to SEQ ID NO: 15

SEQ ID NO: 127 - DNA, 2539 bp, Cricetulus griseus; identical to SEQ ID NO: 16

SEQ ID NO: 128 - DNA, 6665 bp, Cricetulus griseus; identical to SEQ ID NO: 17

SEQ ID NO: 129 - DNA, 4473 bp, Cricetulus griseus; identical to SEQ ID NO: 18

SEQ ID NO: 130 - DNA, 5683 bp, Cricetulus griseus; identical to SEQ ID NO: 19

SEQ ID NO: 131 - DNA, 4534 bp, Cricetulus griseus; identical to SEQ ID NO: 20

SEQ ID NO: 132 - DNA, 166 bp, Cricetulus griseus; identical to SEQ ID NO: 21

SEQ ID NO: 133 - DNA, 976 bp, Cricetulus griseus; identical to SEQ ID NO: 22

(19) United States
(12) Patent Application Publication
(10) Pub. No.: US 2013/0034875 A1
(43) Pub. Date: Feb. 7, 2013

(54) DNA CONSTRUCT, AND PROCESS FOR PRODUCTION OF RECOMBINANT CHO CELL USING SAME

(71) Applicants: Shuji SONEZAKI, Fukuoka (JP); Yumi OGAMI, Fukuoka (JP); Yoshimasa YAMANA, Kanagawa (JP); Junya NARITA, Kanagawa (JP); Masako NAKAMURA, Fukuoka (JP)

(72) Inventors: Shuji SONEZAKI, Fukuoka (JP); Yumi OGAMI, Fukuoka (JP); Yoshimasa YAMANA, Kanagawa (JP); Junya NARITA, Kanagawa (JP); Masako NAKAMURA, Fukuoka (JP)

(73) Assignee: TOTO LTD., KITAKYUSHU-SHI (JP)

(21) Appl. No.: 13/441,196

(22) Filed: Apr. 6, 2012

Related U.S. Application Data
(63) Continuation-in-part of application No. 13/498,006, filed on Jul. 5, 2012.

(30) Foreign Application Priority Data
Oct. 1, 2009 (JP) 2009-229722
Mar. 8, 2011 (JP) 2011-050748
Mar. 8, 2012 (JP) 2012-051980

(51) Int. Cl. C12N 15/85; C12P 21/00; C12N 5/10

(52) U.S. Cl. 435/69.1; 435/320.1; 435/461; 435/358

(57) ABSTRACT

Disclosed is a DNA construct that is useful for efficient generation of recombinant CHO cells useful for the production of target proteins. The DNA construct is a construct comprising, from a 5′ end toward a 3′ end, a first homologous DNA fragment, a target protein gene, and a second homologous DNA fragment. The first and second homologous DNA fragments have homology allowing for homologous recombination with a part of a hypoxanthine-phosphoribosyltransferase enzyme (hprt) locus in a CHO cell genome and have a chain length of not less than 1 kbp.

### FIELD OF INVENTION

The present invention relates to a DNA construct useful for efficient generation of recombinant CHO cells and a process for generating recombinant CHO cells using the same.

### BACKGROUND ART

Various recombinant protein production systems using procaryotes or eucaryotes as host cells are known. In recombinant protein production systems using mammal cells as host cells, proteins derived from higher animals including humans can be subjected to post-translational modifications such as glycosylation, folding, and phosphorylation in a similar manner to those produced in vivo,

The post-translational modification is necessary for the reproduction of physiological activities of native proteins by recombinant proteins, and protein production systems using mammal cells as host are commonly used in recombinant protein production systems used in medicaments that are particularly required to have such physiological activities.

At the present time, two protein production systems, CHO-DHFR systems and GS-NS0 systems, may be mentioned as main protein production systems using mammal cells that are used in productions on a commercial scale. In these production systems, clones that can realize increased copy numbers of a plasmid vector integrated into chromosomes are screened by combining a selective marker contained in the plasmid vector with a proper drug selection process. In particular, in CHO-DHFR systems, cell clones, of which the expression level has been increased by a factor of several tens, can be screened by a two-stage screening process using a selective drug Methotrexate.

However, it is known that the above protein production systems suffer from problems, for example, that the expression level of the target protein is not simply always increased proportionally with the number of copies of the amplified plasmid vector and that a lot of time is taken for screening of cell clones having an increased expression level. Further, it is reported that, when, after screening of cell clones having an increased expression level, the culture of selected cell clones is continued in a medium free from a selective drug, a reduction or disappearance of the expression level is observed in most of the clones (PTL 1: JP 2002-541854T, NPL 1: Kim, N. S. (1998) Biotechnol. Bioeng., 60, 679-688).

Further, in target protein production systems using mammal cells, in general, a target protein is produced by introducing a vector containing a target protein gene into host cells, screening cell clones in which the vector has been integrated into chromosomes, and further culturing the cell clones under proper culture conditions.

The integration into chromosomes often occurs at random positions, and the target protein expression level varies depending upon the cell clones obtained. Further, some cell clones suffer from problems, for example, that they do not express the target protein. To overcome this drawback, a method is adopted in which a number of clones are selected according to a recombinant protein expression level, and favorable clones are selected. This screen process, however, is very troublesome and takes a lot of work. Various processes have been reported for avoiding such a troublesome task and quickly selecting favorable clones.

For example, a technique is disclosed in which a vector is integrated into a specific chromosomal position of mouse cells (PTL 2: JP 9 (1997)-510865A). Homologous recombinant cell clones are produced in a recombinant cell clone pool by a vector loaded with a sequence having a base sequence homologous to immunoglobulin γ2A locus. A target chromosomal position is previously identified as a position that, when a foreign gene is integrated, can provide a higher expression level than random integration. Accordingly, when homologous recombinant cell clones having a high expression level are present with given frequency in a recombinant cell clone pool as a screening object, work in screening according to the expression level can be reduced.

A technique for the utilization of a marking plasmid is also disclosed (PTL 3: JP 2001-516221A). Clones having a high expression level of a marker gene present within a marking plasmid are previously selected from a cell clone population obtained by random recombination of the marking plasmid. Next, target protein producing clones that have inherited an expression level of the marker gene are obtained by selecting cell clones in which site-specific recombination has occurred between the plasmid vector having the target protein gene and the randomly integrated marking plasmid sequence.

The above technique is advantageous in reducing work necessary for selecting clones having a high expression level. However, for example, when recombinant cells are continuously cultured without the addition of a selective drug, it is unpredictable whether or not the clones thus obtained can stably maintain the expression level for a long period of time.

In the production of medicinal proteins on a commercial scale, it is important that the expression of proteins is stably maintained at a high level. In particular, stably maintaining the expression level is important from the viewpoint of cost, as well as from the viewpoint of proving identity and safety as medicinal proteins. In order to use recombinant protein producing cells in production on a commercial scale, it is necessary to increase a culture scale of producing cell clones. It is estimated that, in general, at least about 60 times of cell divisions are necessary from clones immediately after the establishment (NPL 2:Brown, M. E. et al. (1992) Cytotechnology, 9, 231-236), and the expression level should be maintained at a constant level.

Further, selective drugs incur an increased culture cost and, at the same time, incurs an increase in cost involved in a purification process provided to avoid a possibility of mixing foreign matter into medicaments. Accordingly, the development of a technique for manufacturing of cell clones that can stably maintain the expression level without the addition of a selective drug has been strongly desired.

Despite the above circumstances, it cannot be said that satisfactory technical studies have been made on the stability of the target protein expression level. Up to now, in many processes for generating protein producing system, the selection of clones has been empirically made based on accumulated data on growth rate and productivity in long-term culture. This empirical clone selection method, however, can hardly realize the acquisition of cell clones that have a stable expression level (NPL 3:Barnes, L. M. et al. (2003) Biotechnology and Bioengineering, 81, 631-639).

A technique has recently been studied in which a target protein gene is specifically integrated into a target gene locus in a cell genome to acquire efficiently recombinant cells that can express a protein for a long period of time in a stable manner.

An hprt gene may be mentioned as one example of the target gene. The hprt gene is known as one of housekeeping genes located on the long arm of X chromosome, for example, in humans. Cells after knockout of the hprt gene are resistant to drugs 6-Thioguanime (6TG) and G418, and, thus, negative selection is easy.

Some of the present inventors have reported that recombinant cells that can stably express proteins in the absence of a selective drug for a long period of time have been acquired by introducing a target protein gene into an hprt locus of a human male-derived HT1080 cell line through a recombinant vector (PTL 5: JP 2007-325571A, NPL 4: Koyama Y Et Al., (2006) Biotechnology And Bioengineering, 95, 1052-1060). It is reported in an experiment of this document that about 10 clones of recombinant cells per 10⁷ cells were acquired by using an about 1-kbp first homologous DNA fragment and an about 1-kbp second homologous DNA fragment as homology arms.

Further, targeting to mouse ES cells is reported as another example of targeting to the hprt locus (PTL 6: JP 5 (1993)-507853A).

However, even when the target is an identical locus, a gene targeting frequency sometimes significantly varies depending upon the type of culture cells. For example, in Porter C. G. Itzhaki J. E, Eur. J. Biochem 218, 273-281 (NPL 5) and Annual Report of the Hiroshima University Research Institute for Radiation Biology and Medicine, vol. 44 (2003) (NPL 6), it is reported that ES cells and HT1080 cells are different from each other in gene targeting frequency and, in somatic cell-derived culture cells, the gene targeting frequency is very low.

On the other hand, CHO cells are utilized as host cells in antibody medicinal protein producing systems, and the construction of a high-level and stable protein producing system using CHO cells have been demanded.

However, there is no report that gene targeting has been done to hprt locus of CHO cells. There are only studies on the use of special cell lines that are deficient in an aprt gene on one chromosome of CHO cells (NPL 11: PNAS, 88, 9488-9502 (1991), NPL 12: Somatic Cell. Mol. Genet., 19, 363-375, NPL 13: PNAS, 86, 4574-4578 (1989)). In these experiments, a cell line in which an aprt gene is present only on one chromosome is used as a host, and 2.6 kbp to 4 kbp-homologous DNA fragments are used as homology arms. As a result, homologous recombinants of approximately several clones to 15 clones per 10⁷ cells are acquired.

There is no report on sequences of genome of an hprt locus of CHO cells except for exons. Accordingly, specific gene targeting to an hprt locus of CHO cells poses a problem that, at the outset, all base sequences other than exons should be analyzed and identified.

Further, CHO cells are female-derived cells and thus have two X chromosomes that have two hprt locuses. Accordingly, when a foreign gene is integrated into both hprt locuses of the chromosomes, CHO cells become resistant to selective drugs such as 6TG. The probability of recombination in such two chromosomes is generally lower than that in male-derived cells. For example, when the probability of recombination in one chromosome is presumed to be 10⁻⁶ while using the efficiency of recombination into an hprt locus of a male-derived HT1080 cell line in Koyama Y Et Al., (2006) Biotechnology And Bioengineering, 95, 1052-1060 (NPL 4) as a reference, the theoretical probability of simultaneous recombination in two chromosomes is 10⁻¹².

Further, in female-derived cells, one of two X chromosomes is inherited paternally and the other is inherited maternally. Accordingly, polymorpholism exists. Homology to genome of homology arms is important in the frequency of gene targeting, and a difference in base sequence sometimes leads to a significant lowering in gene targeting frequency (NPL 7: Datt, A. et al., (1997) Proc. Natl. Acad. Sci. U.S.A. 94, 9757-9762, NPL 8: Selva E. M. et al., (1995) Genetics. 139, 1175-1188, NPL 9: Riele H. et al., (1992) Proc. Natl. Acad. Sci. U.S.A. 89, 5128-5132, NPL 10: Deng C, D, Capecchi M, R (1992) Mol. Cell. Biol. 12, 3365-3371). Accordingly, in female-derived culture cells such as CHO cells, the influence of polymorpholism of X chromosomes can also render the frequency of acquisition of recombinant cells lower than that in male-derived culture cells.

Thus, the development of a technique for efficiently generating recombinant CHO cells that express a target protein gene is still demanded.

Further, dhfr deficient CHO cells are a cell line deficient in a DNA de novo synthesis capability and survive through the procurement of their own DNA by salvage of hypoxanthine and thymidine in the medium. An hprt gene function is indispensable for a salvage pathway. Accordingly, the integration of a vector into an hprt locus leads to a lethal state (NPL 17: Carcinogenesis, 20, 215-220 (1999)). This poses a problem that the hprt locus cannot be used as a target site for foreign gene integration.

A method has been disclosed that expresses a two function selectable fusion gene obtained by fusing a positive selection gene and a negative selection gene to each other. A dhfr gene is mentioned as an example of the positive selection gene, and an hprt gene is mentioned as an example of the negative selection gene (PTL 7: JP H06-504432A).

When this method is adopted, a dhfr-hprt fusion gene is mounted on a vector and is introduced into cells. Since, however, in dhfr gene deficient CHO cells, hprt on chromosomes is originally in an active state, the screening of recombinant cells can be carried out only based on hypoxanthine non-requirement. In CHO cells, random insertion occurs at a frequency of 4000 times that of homologous recombination (NPL 13: PNAS, 86, 4574-4578 (1989)). There is little or no likelihood that, even when a dhfr-hprt fusion gene together with a homology arm homologous to a chromosomal hprt locus is introduced into dhfr gene deficient CHO cells, cells subjected to homologous recombination with the hprt locus can be acquired.

Thus, the development of a technique that can realize screening of cells obtained by integrating a target protein gene into an hprt locus of dhfr gene deficient CHO cells is still demanded.

### CITATION LIST

### Patent Literature

[PTL 1] JP 2002-541854A
[PTL 2] JP 9 (1997)-510865A
[PTL 3] JP 2001-516221A
[PTL 4] WO 2004/022741A
[PTL 5] JP 2007-325571A
[PTL 6] JP 5 (1993)-507853A
[PTL 7] JP 6 (1994)-504432A

### Non Patent Literature

[NPL 1] Kim, N. S. (1998) Biotechnol. Bioeng., 60, 679-688.
[NPL 2] Brown, M. E. et al. (1992) Cytotechnology, 9, 231-236.
[NPL 3] Barnes, L. M. et al. (2003) Biotechnology and Bioengineering, 81, 631-639.

[NPL 4] Koyama Y Et Al., (2006) Biotechnology And Bioengineering, 95, 1052-1060
[NPL 5] Porter C. G. Itzhaki J. E, Eur. J. Biochem 218, 273-281
[NPL 6] Annual Report of the Hiroshima University Research Institute for Radiation Biology and Medicine, vol. 44 (2003)
[NPL 7] Datt, A. et al., (1997) PNAS, U.S.A. 94, 9757-9762
[NPL 8] Selva E. M. et al., (1995) Genetics. 139, 1175-1188
[NPL 9] Riele H. et al., (1992) Proc. Natl. Acad. Sci. U.S.A. 89, 5128-5132
[NPL 10] Deng C, D, Capecchi M, R (1992) Mol. Cell. Biol. 12, 3365-3371
[NPL 11] PNAS, 88, 9488-9502(1991)
[NPL 12] Somatic Cell. Mol. Genet., 19, 363-375
[NPL 13] PNAS, 86, 4574-4578(1989)
[NPL 14] Mol Gen. Genet., 212, 301-309(1988)
[NPL 15] Molecular Biology Reports, 31, 85-90(2004)
[NPL 16] Biotechnology and Bioengineering, 91, 1-11(2005)
[NPL 17] Carcinogenesis, 20, 215-220(1999)

### SUMMARY OF INVENTION

The present inventors have found that recombinant CHO cells can be generated with significantly high frequency by determining a complete DNA sequence including an intron in an hprt gene of CHO cells and further introducing a target protein gene into CHO cells by a specific DNA construct containing a homologous DNA fragment of the hprt gene. The present invention has been made based on such finding.

Accordingly, an object of the present invention is to provide a DNA construct useful for efficient generation of recombinant CHO cells and a process for generating recombinant CHO cells using the same.

According to one aspect of the present invention, there is provided a DNA construct comprising, from a 5′ end toward a 3′ end, a first homologous DNA fragment, a target protein gene, and a second homologous DNA fragment,

the first and second homologous DNA fragments having homology allowing for homologous recombination with a part of a hypoxanthine-phosphoribosyltransferase enzyme (hprt) locus in a CHO cell genome and having a chain length of not less than 1 kbp.

According to another aspect of the present invention, there is provided a process for generating recombinant CHO cells, the process comprising introducing the vector containing the DNA construct into CHO cells.

According to the present invention, recombinant CHO cells that express target protein genes can be acquired with significantly high frequency.

### BRIEF DESCRIPTION OF DRAWINGS

FIG. 1 is a typical diagram of an hprt locus of a CHO-K1 cell line in Example 1. Nos. 1 to 9 represent exons 1 to 9 of the hprt gene, respectively, and sequences between the exons represent introns 1 to 8.

FIG. 2 is a typical diagram of a vector for homologous recombination used in Example 2. FIG. 2 (A) shows a vector containing a homologous DNA fragment derived from an hprt gene of CHO cells. FIG. 2 (B) shows a vector containing a homologous DNA fragment derived from human HT1080 cells.

FIG. 3 (A) is a typical diagram showing a relationship among an hprt gene region of CHO cells, a vector for homologous recombination, and DNA as an index of genome PCR of recombinant CHO cells in Example 4. FIGS. 3 (B) and 3 (C) are diagrams showing the results of PCR in Example 4.

FIG. 4 (A) is a typical diagram showing a relationship among an hprt gene region of CHO cells, a vector for homologous recombination, and DNA as an index of Southern hybridization in Example 5. FIG. 4 (B) shows the results of Southern hybridization in Example 5.

FIG. 5 is a graph showing the amount of antibody produced by recombinant CHO cells acquired in Example 6.

FIG. 6 is a typical diagram of a vector for homologous recombination having a 2.5-kbp homologous DNA fragment used in Example 7.

FIG. 7 (A) is a typical diagram showing a relationship among a hprt gene region of CHO cells, a vector for homologous recombination, and DNA as an index of genome PCR of recombinant CHO cells in Example 7. FIGS. 7 (B) and (C) show the results of PCR in Example 7.

FIG. 8 (A) is a typical diagram showing a relationship among a hprt gene region of CHO cells, a vector for homologous recombination, and DNA as an index of Southern hybridization in Example 7. FIG. 8 (B) is a diagram showing the results of Southern hybridization in Example 7.

FIG. 9 (A) is a typical diagram for explaining details of genome PCR for the confirmation of remaining of a wild-type hprt gene in CHO cells. The drawing located left in FIG. 9 (A) shows PCR in wild-type CHO cells, the drawing located at the center of FIG. 9 (A) shows PCR in heterojunction-type recombinant CHO cells, and the drawing located right in FIG. 9 (A) shows PCR in homojunction-type recombinant CHO cells. FIG. 9 (B) shows the results of PCR in Example 8.

FIG. 10 is a typical diagram of a vector for homologous recombination that has a 2.5-kbp homologous DNA fragment and a CHO endogenous promoter used in Example 9.

FIG. 11 is a diagram showing the productivity of antibody in Example 9 where acquired recombinant CHO cells are subcultured for a long period of time.

FIG. 12 is a typical diagram showing a vector for homologous recombination used in Example 10 that has a 2.5-kbp homologous DNA fragment.

FIG. 13 is a typical diagram showing a CHO recombinant vector having MAR derived from a human hprt gene intron and a chromosome comprising the vector integrated into a CHO hprt locus used in Example 11. FIG. 13 (A) shows a vector having an MAR non-transcriptional structure and a chromosome structure after the integration. FIG. 13 (B) shows a vector having a structure that MAR is transcribed by transcription of an hprt locus and a chromosome structure after the integration. FIG. 13 (C) shows a vector having a structure that MAR is transcribed by a CMV promoter and a chromosome structure after the integration.

FIG. 14 is a graph showing stability in antibody production of CHO cells acquired by a CHO recombinant vector having MAR used in Example 11.

FIG. 15 is a typical diagram showing a CHO recombinant vector having a homology arm length of 200b×2 (A) and a CHO recombinant vector having a homology arm length of 500b×2 (B) used in Example 12.

FIG. 16 is a typical diagram showing a CHO recombinant vector having a homology arm length of 5 kb×2 used in Example 13.

FIG. 17 is a typical diagram of a dhfr deficient CHO cell hprt homologous recombination vector used in Example 14. FIG. 17 (A) shows a vector that has a homologous DNA fragment derived from an hprt gene of dhfr deficient CHO cells and has a dhfr gene as a selective marker gene. FIG. 17 (B) shows a vector that has a homologous DNA fragment derived from an hprt gene of dhfr gene deficient CHO cells, has a dhfr gene as a selective marker gene, and contains an antibody expression cassette.

FIG. 18 (A) is a typical diagram showing a relationship among an hprt gene region of dhfr gene deficient CHO cells, a homologous recombination vector, and DNA as an index of genome PCR of recombinant cells in Example 16. FIG. 18 (B) is a typical diagram for explaining details of genome PCR for the confirmation of remaining of a wild-type hprt gene in recombinant dhfr gene deficient CHO cells. The drawing located left in FIG. 18 (B) shows PCR in wild-type dhfr gene deficient CHO cells. The drawing located at the center of FIG. 18 (B) shows PCR in heterozygous-type recombinant cells, and the drawing located right in FIG. 18 (B) shows PCR in homozygous-type recombinant cells. FIG. 18 (C) is a diagram showing the results of PCR in Example 16.

FIG. 19 is a typical diagram of an hprt locus of dhfr deficient CHO cells. Nos. 1 to 9 represent exons 1 to 9 of the hprt gene, respectively, and sequences between the exons represent introns 1 to 8.

FIG. 20 (A) is a typical diagram showing a relationship among an hprt gene region of DXB11, a homologous recombination vector, and DNA as an index of genome PCR of recombinant cells in Example 17. FIG. 20 (B) shows the results of PCR in Example 17.

FIG. 21 is a graph showing the results of antibody production by a dhfr deficient CHO hprt recombinant antibody expression clone manufactured in Example 18.

FIG. 22 is a graph showing the results of a long-term stability test on the specific productivity of an antibody by a dhfr deficient CHO hprt recombinant antibody expression clone manufactured in Example 18.

### DESCRIPTION OF EMBODIMENTS

DNA Construct

One feature of the DNA construct according to the present invention is to comprise two homologous DNA fragments that have homology allowing for homologous recombination with a part of hprt locus in a CHO cell genome and each have a chain length of not less than 1 kbp. It is surprising that, according to the DNA construct, recombinant CHO cells can be acquired with significantly high frequency despite the fact that CHO cells have two X chromosomes. According to the DNA construct of the present invention, as demonstrated in Example 10 which will be described later, 130 recombinant cells are obtained per 1×10⁻⁷ cells. As described above, since the estimated probability of simultaneous recombination in hprt locuses of the two chromosomes is theoretically 10⁻¹², this result is a surprising fact.

In the DNA construct according to the present invention, a part of the hprt locus is a target region of homologous recombination. The adoption of a part of the hprt locus as the target region is advantageous in stably expressing the target protein gene on a high level. Further, the integration of the DNA construct into the target region is also preferred from the viewpoints of inhibiting the transcription and expression of the hprt gene to inactivate the function of the hprt gene and efficiently acquiring recombinant cells by negative selection using 6-TG or the like.

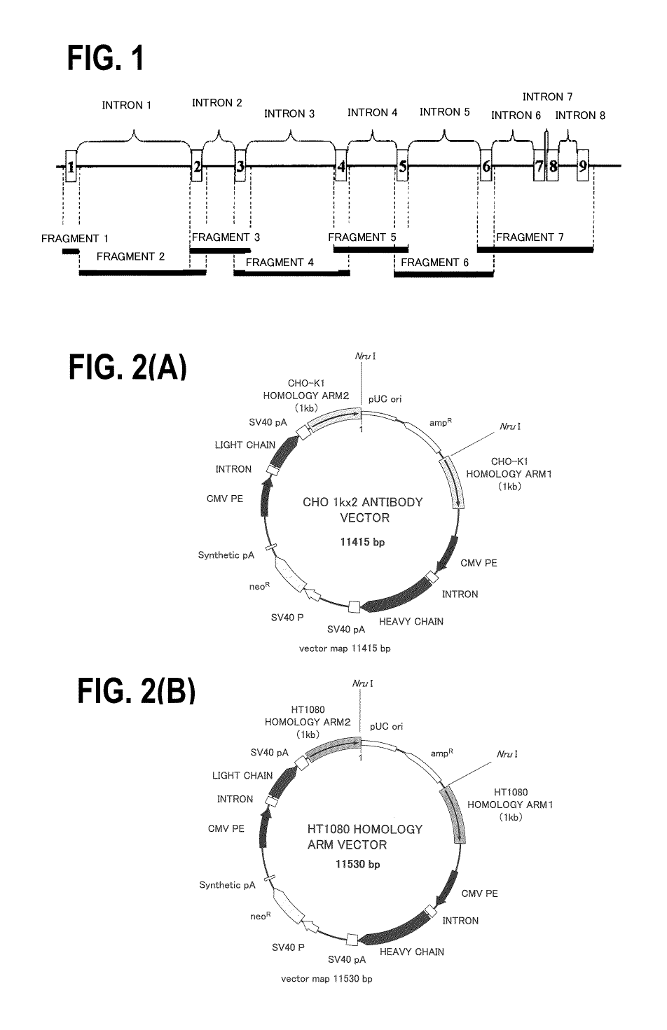

The target region in the present invention may be properly determined in the hprt locus as long as the expression of the target protein gene is not inhibited. When the chain length and the like of homologous DNA fragments necessary for homologous recombination are taken into consideration, the target region is preferably a region containing at least a part of introns of the hprt gene. For introns, the present inventors have now determined base sequences. Specifically, introns 1 to 8 shown in FIG. 1 which will be described later, may be mentioned that have a base sequence represented by any of SEQ ID Nos. 15 to 22.

The target region according to the present invention may contain the whole or a part of exons adjacent to the introns. Specifically, exons 1 to 9 shown in FIG. 1 may be mentioned as the exon. Base sequences thereof can be acquired by access to known database, for example, in National Center for Biotechnology Information of USA.

In the present invention, homologous DNA fragments homologous to a desired region in the hprt locus may be properly constructed by a person having ordinary skill in the art based on information about the arrangements of and their base sequences of introns and exons in the hprt locus shown in FIG. 1.

The DNA construct according to the present invention having the homologous DNA fragments can be integrated into a desired target region in the hprt locus. Embodiments of the integration include (1) a type in which exons are divided by the integration of the DNA construct, (2) a type in which one or more exons are deleted by the integration of the DNA construct, (3) a type in which one exon is amplified to two by the integration of the DNA construct and the DNA construct is inserted into between the two exons, (4) a type in which introns are divided by the integration of the DNA construct, (5) a type in which one or more introns are deleted by the integration of the DNA construct, (6) a type in which one intron is increased to two by the integration of the DNA construct and the DNA construct is inserted into between the two introns.

When the chain length necessary for homologous recombination is taken into consideration, as described above, the DNA fragment according to the present invention is preferably homologous to a region containing at least a part of introns of the hprt gene. Accordingly, in one embodiment of the present invention, the first homologous DNA fragment and the second homologous DNA fragment according to the present invention comprise a base sequence described in any of SEQ ID Nos. 15 to 22 or a partial sequence thereof.

The lower limit of the chain length of the partial sequence is 1 bp, and the upper limit can be properly regulated in a range of chain lengths of base sequences described in any of SEQ ID Nos. 15 to 22.

The homology between the homologous DNA fragments and the hprt locus may be properly determined by taking into consideration of the efficiency of homologous recombination but is preferably not less than 99.0%, more preferably not less than 99.9%, still more preferably 100%. The homology may be properly determined, for example, by analysis with DNA sequencer or the like.

Further, the homologous DNA fragments have a chain length of not less than 1 kbp, and this chain length is preferred in achieving homologous recombination of the DNA construct with significantly high frequency. The lower limit of the chain length of the homologous DNA fragments is preferably not less than 2.5 kbp. The upper limit of the chain length of the homologous DNA fragments is not more than 7.5 kbp, preferably not more than 5 kbp. The upper limit and the lower limit of the chain length of the homologous DNA fragments may be properly combined to define the chain length range of the homologous DNA fragments. Specifically, the chain length is preferably 1 kbp to 7.5 kbp, more preferably 1 kbp to 5 kbp, most preferably 2.5 kbp to 5 kbp. When the chain length is in the above-defined range, the frequency of acquisition of the recombinant cells can be kept good while achieving homologous recombination of the DNA construct with significantly high frequency.

In the DNA construct according to the present invention, the target protein gene preferably codes for a protein useful as a medicine. cDNA-derived sequences or structural genes containing natural introns derived from genome DNA may be suitably utilized as a target protein gene. Specific examples of target proteins include antibodies, enzymes, cytokines, hormones, coagulation factors, regulatory proteins, and receptors. Preferred are monoclonal antibodies, polyclonal antibodies, erithropoietins, and tissue-specific plasminogen activators or granulocyte colony activators.

Preferably, the target protein gene is integrated as an expression unit containing elements necessary for expression of such as promoter sequences and transcriptional termination signal sequences into the hprt locus. In one embodiment of the present invention, the target protein gene is disposed as an expression unit containing at least a promoter sequence and a transcriptional termination signal sequence in the DNA construct.

A construction may also be adopted in which the element necessary for expression may be endogenous in CHO cells. This embodiment falls within the scope of the present invention.

The promoter or the transcriptional termination signal may be properly determined depending, for example, upon the type and properties of the target protein gene. Examples of suitable promoter sequences include CMV promoters and SV40 promoters. Examples of suitable transcriptional termination signal sequences include BGH poly A signal sequences and SV40 poly A signal sequences.

For example, regulatory elements for efficient expression of a target gene (for example, enhancers, IRES (internal ribosome entry site) sequences, LoxP sequences, FRT sequences or other recombinant enzyme recognition sequences) may be properly selected and used as the element necessary for expression other than the promoter sequence and the transcriptional termination signal sequence. The regulatory element may be disposed at a proper position in the expression unit depending upon the properties. The element necessary for expression may be properly selected by taking into consideration, for example, the productivity of the target protein.

Preferably, the DNA construct comprises, in addition to the above elements, a positive selective marker gene. The positive selective marker gene may be properly disposed in the DNA construct as long as the homologous recombination is not inhibited. Examples of suitable positive selective marker genes include neomycin-resistant genes, hygromycin resistant genes, Zeocin resistant genes, dihydrofolate reductase genes, and glutamine synthase genes. The use of the positive selective marker gene is advantageous in that, in the selection of recombinant CHO cells, both positive selection and negative selection by inactivation of the hprt gene can be applied and, thus, false-positive clones can be significantly reduced.

Vector

The DNA construct according to the present invention can be integrated into the vector, followed by introduction into a CHO cell genome. The vector system is not particularly limited as long as the DNA construct can be integrated into the CHO cell genome by a homologous recombination reaction. Preferred are plasmid vectors, cosmid vectors, phage vectors, or artificial chromosome vectors.

The DNA construct according to the present invention and the vector comprising the DNA construct are suitably constructed by a combination of a restriction enzyme cleaving reaction and a ligation reaction. A DNA construct and a vector comprising the DNA construct can be constructed, for example, by incorporating a restriction enzyme recognition sequence at both ends of each constituent unit, performing a cleaving reaction with a restriction enzyme for the recognition sequence, removing unnecessary DNA sequences (for example, operating sequences within E. coli) by taking-off of gel, and performing a ligation reaction of the resultant unit.

The vector DNA constructed by the ligation reaction is purified, for example, by phenol-chloroform extraction and can be grown in host cells such as E. coli. or yeasts selected while taking into consideration of, for example, the type of vectors.

Preferably, the vector according to the present invention mounts a CHF1α promoter operatively ligated to the target protein gene. The expression of a foreign genes integrated into the CHO hprt locus occurs through the inactivation of the promoter. When a vector has the structure that causes expression of the target protein gene by the CHEF1α promoter endogenous in CHO, stable expression in the CHO hprt locus is possible.

Preferably, in the vector according to the present invention, after the integration of the vector into a chromosome, a nucleus/matrix adhesion region (MAR) derived from the first intron of the human hprt gene is maintained in such structure that MAR per se is transcribed by transcription that originally occurs at the integration site. When MAR of the human hprt gene intron is mounted on the vector in the above structure, recombinant CHO having expression stability can be efficiently generated.

The present applicants have previously reported that a foreign gene integrated into an hprt locus is stably expressed in an HT1080 cell line, a human-derived cell line (PTL 4: WO 2004/022741 and NPL 5: Porter C. G. Itzhaki J. E, Eur. J. Biochem 218, 273-281). Further, it is known that a stabilizing factor called a nucleus/matrix adhesion region (MAR) is present in the first intron of the human hprt gene (NPL 14). From these facts, it is estimated that the stabilization of expression in the human hprt gene is realized by the contribution of MAR present around the integration site.

By the way, the hprt gene is a gene that is common in many mammals including CHO. In the hprt locus of CHO, it is estimated that difficulties are encountered in selecting integration cells. Accordingly, examples of cases where the hprt locus has been used as vector integration sites are not known. It is common for a person having ordinary skill in the art to consider that, when a foreign gene is integrated into the hprt locus of CHO, stable expression is possible as in human cells. However, the intron sequence of the hprt locus of CHO is utterly different from that of the hprt gene of human, and a surprising fact that MAR is absent in introns has been elucidated for the first time by the present inventors. In fact, as demonstrated in Example 11, the foreign gene integrated into the hprt locus of CHO could not be stably expressed.

From the above results, when a foreign gene is integrated into the hprt locus of CHO, a person having ordinary skill in the art would consider that, in CHO, stable expression is possible as in human cells by acquiring MAR of the human hprt gene intron, mounting MAR on a CHO recombinant vector, and integrating the vector into the hprt locus of CHO. In line with this way of thinking, related art are known that partial stabilization has been realized by mounting MAR on a vector and randomly integrating the vector into a chromosome of CHO or holding the vector as an extrachromosomal plasmid (NPL 14: Mol Gen. Genet., 212, 301-309 (1988) and NPL 15: Molecular Biology Reports, 31, 85-90 (2004)). In these related art, a structure in which MAR is mounted on the vector at its position where MAR per se is not transcribed (NPL 14) or a structure in which MAR per se is also transcribed by a promoter disposed with an expectation of the target protein gene transcription (NPL 15) is adopted.

However, as demonstrated in Example 11 which will be described later, unexpectedly, in the hprt locus of CHO, these structures offer no significant effect and thus are unsatisfactory for long-term stable expression. It has been found that, in order to achieve stable expression in the hprt locus of CHO, as demonstrated in Example 11, a necessary structure is that MAR mounted on the vector is transcribed only by transcription that originally occurs at the vector integrated site.

Recombinant CHO Cells/Generation Process

The recombinant CHO cells according to the present invention can be suitably generated by introducing the vector into CHO cells.

Accordingly, the recombinant CHO cells according to the present invention comprise an exogenous target protein gene integrated into hprt locus. In a preferred embodiment of the present invention, in the recombinant CHO cells, the function of the hprt gene is inactivated. The recombinant CHO cells are advantageous in stably producing target proteins such as antibodies on a high level.

Introduction of Vector

Commonly used methods are suitable for the introduction of the vector. Examples of such methods include a calcium phosphate method, an electroporation method, a microinjection method, DEAE-dextran method, a method using a liposome reagent, and a lipofection method using a cationic lipid. When the vector is cyclic, a method may also be adopted in which the vector is linearized by a conventional method and the linearized vector is then introduced into cells.

Depending upon properties of the vector or the target protein, in consideration of acquisition efficiency and the like of recombinant cells, a site-specific introduction system utilizing a recombinant enzyme such as a Cre/LoxP system or an Flp/FRT system may also be properly applied. This embodiment also falls within the scope of the present invention.

Screening of Cell Line

In the generation process according to the present invention, after the introduction, screening of recombinant CHO cells is preferably carried out. The step of screening can be carried out by negative selection based on inactivation of the hprt gene. When a positive marker gene has been introduced into recombinant CHO cells, screening of cells can be carried out with high accuracy by a combination of the negative selection with the positive selection.

In addition to the above screening methods, for example, a promoter trap method and a poly A trap method may be properly used in combination.

Further, in the generation method according to the present invention, culture under serum-free conditions is possible by acquiring recombinant CHO cells and then naturalizing the recombinant CHO cells to a chemical defined medium or the like. Conditions for the naturalization may be properly determined depending upon the state of recombinant cells.

Process for Producing Target Protein

According to another aspect of the present invention, there is provided a process for producing a target protein, the process comprising providing the recombinant CHO cells and culturing the cells to produce a target protein. According to this process, the target protein can be acquired in an efficient and stable manner.

The medium used in the step of culture may be properly selected from conventional media depending upon the state of recombinant CHO cells but is preferably serum-free medium. When the culture cost and the purification cost are taken into consideration, preferably, the medium is not supplemented with a selective drug. Examples of suitable media include chemical defined media.

Conventional culture methods such as batch culture method, fed batch culture method, and reflux culture method are applicable as the culture method.

Various methods used in the generation of recombinant CHO cells are described in more detail, for example, in F. M. Ausubel, Current Protocols in Molecular Biology, John Wiley & Sons, New York, (1989), the disclosure of which is incorporated by reference herein.

Embodiment where selective marker gene is dihydrofolate reductase gene (dhfr)

DNA Construct

One feature of the DNA construct according to the present invention is to comprise two homologous DNA fragments that have homology allowing for homologous recombination with a part of hprt locus in a CHO cell genome and each have a chain length of not less than 1 kbp. It is surprising that, according to the DNA construct, recombinant CHO cells can be acquired with significantly high frequency despite the fact that CHO cells have two X chromosomes. According to the DNA construct of the present invention, as demonstrated in Example 16 which will be described later, 4 recombinant cells are obtained per 2×10⁵ cells. Since the estimated probability of simultaneous recombination in hprt locuses of the two chromosomes is theoretically 10⁻¹² in the state of the art at the time of filing of the present application, this result can be said to be a surprising fact.

Further, another feature of the DNA construct according to the present invention is to comprise a dihydrofolate reductase gene (dhfr) as a positive selective marker gene. The dhfr gene may be properly disposed in the DNA construct as long as the homologous recombination is not inhibited. The use of the dhfr gene is advantageous in that lethality attributable to a combination of a de novo synthesis capability with inactivation of the hprt gene can be avoided and, further, in that, in the selection of recombinant CHO cells, both positive selection using hypoxanthine and thymidine non-requirement as an index and negative selection by inactivation of the hprt gene can be applied and, thus, false-positive clones can be significantly reduced.

In the DNA construct according to the present invention, a part of the hprt locus is a target region of homologous recombination. The adoption of a part of the hprt locus as the target region is advantageous in stably expressing the target protein gene on a high level. Further, the integration of the DNA construct into the target region is also preferred from the viewpoints of inhibiting the transcription and expression of the hprt gene to inactivate the function of the hprt gene and efficiently acquiring recombinant cells by negative selection using 6-TG or the like.

The target region in the present invention may be properly determined in the hprt locus as long as the expression of the target protein gene is not inhibited. When the chain length and the like of homologous DNA fragments necessary for homologous recombination are taken into consideration, the target region is preferably a region containing at least a part of introns of the hprt gene. For introns, the present inventors have now determined base sequences. Specifically, introns 1 to 8 having a base sequence represented by any of SEQ ID Nos. 126 to 133 that are shown in FIG. 16 and will be described later, may be mentioned.

The target region according to the present invention may contain the whole or a part of exons adjacent to the introns. Specifically, exons 1 to 9 shown in FIG. 16 may be mentioned as the exon. Base sequences thereof can be acquired by access to known database, for example, in National Center for Biotechnology Information of USA.

Embodiments of the integration include (1) a type in which exons are divided by the integration of the DNA construct, (2) a type in which one or more exons are deleted by the integration of the DNA construct, (3) a type in which one exon is amplified to two by the integration of the DNA construct and the DNA construct is inserted into between the two exons, (4) a type in which introns are divided by the integration of the DNA construct, (5) a type in which one or more introns are deleted by the integration of the DNA construct, (6) a type in which one intron is amplified to two by the integration of the DNA construct and the DNA construct is inserted into between the two introns.

When the chain length necessary for homologous recombination is taken into consideration, as described above, the DNA fragment according to the present invention is preferably homologous to a region containing at least a part of introns of the hprt gene. Accordingly, in one embodiment of the present invention, the first homologous DNA fragment or the second homologous DNA fragment according to the present invention comprises a base sequence described in any of SEQ ID Nos. 126 to 133 or a partial subsequence thereof.

The lower limit of the chain length of the partial sequence is 1 bp, and the upper limit can be properly regulated in a range of chain lengths of base sequences described in any of SEQ ID Nos. 126 to 133.

The homology between the homologous DNA fragments and the hprt locus may be properly determined by taking into consideration of the efficiency of homologous recombination but is preferably not less than 99.0%, more preferably not less than 99.9%, still more preferably 100%. The homology may be properly determined, for example, by analysis with DNA sequencer or the like.

Further, the homologous DNA fragments have a chain length of not less than 1 kbp, and this chain length is preferred in achieving homologous recombination of the DNA construct with significantly high frequency. The chain length of the homologous DNA fragments may be properly regulated while taking into consideration, for example, recombination efficiency and is more preferably not less than 2.5 kbp. The upper limit of the chain length of the homologous DNA fragments is preferably not more than 7.5 kbp, most preferably not more than 5 kbp, from the viewpoint of avoiding the inhibition of efficient introduction of the target protein gene. The upper limit and the lower limit of the chain length of the homologous DNA fragments may be properly combined to define the chain length range of the homologous DNA fragments. Specifically, the chain length is preferably 1 kbp to 7.5 kbp, more preferably 1 kbp to 5 kbp, most preferably 2.5 kbp to 5 kbp. When the chain length is in the above-defined range, the frequency of acquisition of the recombinant cells can be kept good while achieving homologous recombination of the DNA construct with significantly high frequency.

In the DNA construct according to the present invention, the target protein gene preferably encodes for a protein useful as a medicine. cDNA-derived sequences or structural genes containing natural introns derived from genome DNA may be suitably utilized as a target protein gene. Specific examples of target proteins include antibodies, enzymes, cytokines, hormones, coagulation factors, regulatory proteins, and receptors. Preferred are monoclonal antibodies, polyclonal antibodies, erythropoietins, and tissue-specific plasminogen activators or granulocyte colony activators.

Preferably, the target protein gene is integrated as an expression unit containing elements necessary for expression such as promoter sequences and transcriptional termination signal sequences into the hprt locus. In one embodiment of the present invention, the target protein gene is disposed as an expression unit containing at least a promoter sequence and a transcriptional termination signal sequence in the DNA construct. A construction may also be adopted in which the element necessary for expression may be endogenous in CHO cells. This embodiment falls within the scope of the present invention.

The promoter or the transcriptional termination signal may be properly determined depending, for example, upon the type and properties of the target protein gene. Examples of suitable promoter sequences include CMV promoters and SV40 promoters. Examples of suitable transcriptional termination signal sequences include BGH poly A signal sequences and SV40 poly A signal sequences.

For example, regulatory elements for efficient expression of a target gene (for example, recombinant enzyme recognition sequences such as enhancers, IRES (internal ribosome entry site) sequences, LoxP sequences, FRT sequences and the like) may be properly selected and used as the element necessary for expression other than the promoter sequence and the transcriptional termination signal sequence. The regulatory element may be disposed at a proper position in the expression unit depending upon the properties. The element necessary for expression may be properly selected by taking into consideration, for example, the productivity of the target protein.

Vector

The DNA construct according to the present invention can be integrated into the vector, followed by introduction into a dhfr gene deficient CHO cell genome. The vector system is not particularly limited as long as the DNA construct can be integrated into the cell genome by a homologous recombination reaction. Preferred are plasmid vectors, cosmid vectors, phage vectors, or artificial chromosome vectors.

The DNA construct according to the present invention and the vector comprising the DNA construct are suitably constructed by a combination of a restriction enzyme cleaving reaction and a ligation reaction. A DNA construct and a vector comprising the DNA construct can be constructed, for example, by incorporating a restriction enzyme recognition sequence at both ends of each constituent unit, performing a cleaving reaction with a restriction enzyme for the recognition sequence, removing unnecessary DNA sequences (for example, operating sequences within E. coli) by taking-off of gel, and performing a ligation reaction of the resultant unit.

The vector DNA constructed by the ligation reaction is purified by, for example, phenol-chloroform extraction and can be grown in host cells such as E. coli. or yeasts selected while taking into consideration, for example, the type of vectors.

In the vector according to the present invention, preferably, a CHEF1α promoter is operatively ligated to and mounted on the target protein gene. The expression of a foreign genes integrated into the CHO hprt locus occurs through the inactivation of the promoter. When a vector structure that causes expression of the target protein gene by the CHEF1α promoter endogenous in CHO is adopted, stable expression in the CHO hprt locus is possible.

dhfr Deficient CHO Cell Line

The cell line which is the subject matter of the present invention is a dhfr gene deficient Chinese hamster cell line, and examples thereof include DG44 and DXB11.

Recombinant dhfr Deficient CHO Cells/Generation Process

The recombinant cells of dhfr deficient CHO according to the present invention can be suitably generated by introducing the vector into dhfr deficient CHO cells. Accordingly, the recombinant cells of dhfr deficient CHO according to the present invention comprise an exogenous target protein gene integrated into an hprt locus. In a preferred embodiment of the present invention, the function of the hprt gene in the recombinant dhfr deficient CHO cells is inactivated. The recombinant dhfr deficient CHO cells are advantageous in stably producing target proteins such as antibodies on a high level.

Introduction of Vector

Commonly used methods are suitable for the introduction of the vector. Examples of such methods include a calcium phosphate method, an electroporation method, a microinjection method, DEAE-dextran method, a method using a liposome reagent, and a lipofection method using a cationic lipid. When the vector is cyclic, a method may also be adopted in which the vector is linearized by a conventional method and the linearized vector is then introduced into cells.

Depending upon properties of the vector or the target protein, in consideration of acquisition efficiency and the like of recombinant cells, a site-specific introduction system utilizing a recombinant enzyme such as a Cre/LoxP system or an Flp/FRT system may also be properly applied. This embodiment also falls within the scope of the present invention.

Screening of Cell Line

In the generation process according to the present invention, after the introduction, screening of recombinant cells is preferably carried out. The step of screening can be carried out by negative selection based on inactivation of the hprt gene. When a dhfr gene has been introduced as a positive marker gene into recombinant cells, screening of cells can be carried out with high accuracy by a combination of the negative selection with the positive selection using hypoxanthine and thymidine non-requirement.

In addition to the above screening methods, for example, a promoter trap method and a poly A trap method may be properly used in combination.

Further, in the generation method according to the present invention, culture under serum-free conditions is possible by acquiring recombinant cells and then habituating the recombinant cells to a chemical defined medium or the like. Conditions for the habituation may be properly determined depending upon the state of recombinant cells.

Process for Producing Target Protein

According to another aspect of the present invention, there is provided a process for producing a target protein, the process comprising providing the recombinant cells and culturing the cells to produce a target protein. According to this process, the target protein can be acquired in an efficient and stable manner.

The medium used in the step of culture may be properly selected from conventional media depending upon the state of recombinant cells and is preferably a serum-free medium. When the culture cost and the purification cost are taken into consideration, preferably, the medium is not supplemented with a selection drug. Examples of suitable media include chemical defined media and the like.

Conventional culture methods such as batch culture, fed batch culture, and reflux culture are applicable as the culture method.

Various methods used in the generation of recombinant cells are described in more detail, for example, in F. M. Ausubel, Current Protocols in Molecular Biology, John Wiley & Sons, New York, (1989), the disclosure of which is incorporated by reference herein.

### EXAMPLES

The present invention is further illustrated by the following Examples that are not intended as a limitation of the invention.

In the following experiments, conditions for reactions such as reactions by restriction enzymes, PCR reactions, and ligation reactions were set by reaction conditions recommended by manufacturers or methods described in Molecular Cloning 2nd Edition; Sambrook et al., Cold Spring Harbor Laboratory Press. For various plasmid vector DNAs obtained, DNA sequences were determined with a DNA sequencer (310 Genetic Analyser Applied Bio Systems, Inc.). Homology arms 1 and 2 correspond to first and second homologous DNA fragments according to the present invention, respectively.

### Example 1

### Acquisition of Information on hprt Locus DNA Sequence of CHO Cells

A CHO-K1 cell line, a Chinese hamster oocyte cell line, obtained from JCRB Cell Bank (Cell No; JCRB9018) was cultured in a CO₂ incubator (37° C., 5% CO₂) using an AMEM medium (composition; Advanced MEM (GIBCO), 5% [v/v] FBS, 1× GlutaMAX (GIBCO)). The culture solution thus obtained was centrifuged to obtain CHO-K1 cell pellets. The pellets were treated with DNA Isolation Kit for Cells and Tissues (Roche Diagnostics K.K.) to obtain genome DNA. Seven DNA fragments of hprt locus of CHO-K1 cells were obtained by PCR (KOD-Plus ver.2, TOYOBO) using the genome DNA as a template. The fragments were as shown in FIG. 1. PCR primers that had been used for the amplification of the 7 fragments were prepared by reference to primer sequences described in Zu Z et al. Mutat Res. 1993. 288(2): 237-48. The primer sequences were as follows.

Fragment 1 sense primer:
(SEQ ID No. 1)
5′- tctgcaggct tcctcctcac accg -3′
Fragment 1 antisense primer:
(SEQ ID No. 2)
5′- acatgtcaag gcaacgccat ttcca -3′
Fragment 2 sense primer:
(SEQ ID No. 3)
5′- tggaaatggc gttgccttga catgt -3′
Fragment 2 antisense primer:
(SEQ ID No. 4)
5′- caccttttcc aaatcctcga -3′
Fragment 3 sense primer:
(SEQ ID No. 5)
5′- agcttatgct ctgatttgaa atcagctg -3′
Fragment 3 antisense primer:
(SEQ ID No. 6)
5′- cttcagtctg ataaaatcta cagtca -3′
Fragment 4 sense primer:
(SEQ ID No. 7)
5′- aagacttgcc cgagatgtca tgaa -3′
Fragment 4 antisense primer:
(SEQ ID No. 8)
5′- ccaagtgagt gattgaaagc acag -3′
Fragment 5 sense primer:
(SEQ ID No. 9)
5′- tgtgtgtatt caagaatatg catg -3′
Fragment 5 antisense primer:
(SEQ ID No. 10)
5′- gctgagaaaa tttaacagta ttttag -3′
Fragment 6 sense primer:
(SEQ ID No. 11)
5′- caaatacaag caagaatttc ccagag -3′
Fragment 6 antisense primer:
(SEQ ID No. 12)
5′- ggacttgaac atctagggag -3′
Fragment 7 sense primer:
(SEQ ID No. 13)
5′- acttaccact taccattaaa tacc -3′
Fragment 7 antisense primer:
(SEQ ID No. 14)
5′- gacaatctat cgaaggctca tagtgc -3′

DNA sequencing was carried out for the 7 fragments (BigDye Terminator v3.1, Applied Biosystems, Inc.).

As shown in FIG. 1, DNA sequences of

intron 1 located between exon 1 and exon 2 (SEQ ID No. 15),

intron 2 located between exon 2 and exon 3 (SEQ ID No. 16),

intron 3 located between exon 3 and exon 4 (SEQ ID No. 17),

intron 4 located between exon 4 and exon 5 (SEQ ID No. 18),

intron 5 located between exon 5 and exon 6 (SEQ ID No. 19),

intron 6 located between exon 6 and exon 7 (SEQ ID No. 20),

intron 7 located between exon 7 and exon 8 (SEQ ID No. 21), and

intron 8 located between exon 8 and exon 9 (SEQ ID No. 22)

of hprt locus in a CHO-K1 cell line were determined.

### Example 2

### Construction of Targeting Vector

In order to integrate an antibody gene into the hprt locus, a vector (a CHO 1k×2 antibody vector) including an antibody gene and represented by FIG. 2(A) was constructed by the following method.

Further, for comparison, a vector (HT1080 homology arm vector) that has the same construction as the CHO 1k×2 antibody vector except for the substitution of a homologous DNA fragment derived from an hprt gene sequence in CHO cells for a homologous DNA fragment derived from a human hprt gene sequence and is represented by FIG. 2(B) was constructed.

Acquisition of DNA Fragment Homologous to Human hprt Gene

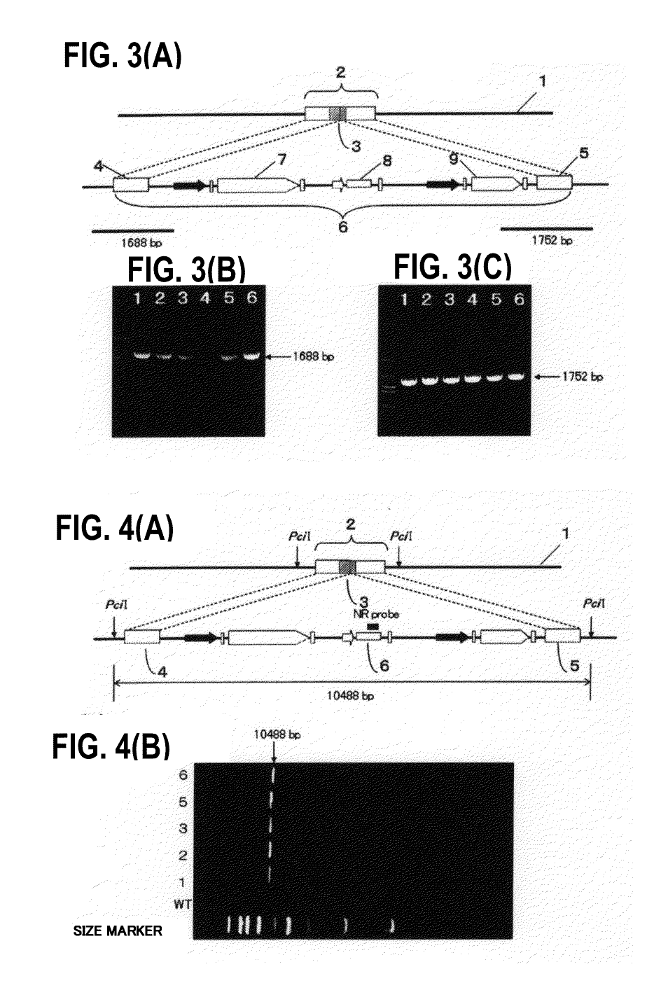

An HT1080 cell line, a human-derived cell line, obtained from JCRB Cell Bank (catalog number: IF050354) was treated with a GFX Genomic Blood DNA Purification Kit (Amersham Biosciences) to obtain genome DNA. Next, DNA fragments (HA1 and HA2) homologous to an hprt gene as a target were cloned by a PCR reaction (KOD-Plus-, TOYOBO) using the genome DNA as a template. As shown in FIG. 3 which will be described later, HA1 and HA2 were set to sequences homologous to a region including exon 3 in the hprt gene. The following primer sequences were used in the PCR reaction.

HA1 sense primer.
(SEQ ID No. 23)
5′-CCTGCAGGTCGCGATTGGTACTTGTTCAGCTTTATTCAAG-3′
HA1 antisense primer:
(SEQ ID No. 24)
5′-GTCGACAAGGACGCGTTCTGATAAAATCTACAGTCATAGGA-3′
HA2 sense primer:
(SEQ ID No. 25)
5′-GTCGACCTCTAGCTAGAGCTTGGCGTAATCATGGTCTCCGGAGA
CTGAAGAGCTATTGTGTGAGTAT-3′
HA2 antisense primer:
(SEQ ID No. 26)
5′-ACATGTTCTCTTAAGTCGCGAAGTAGTGTTATGATGTATGGGCA
TA-3′

In the PCR reaction, a recognition site of restriction enzymes Sse 8387I and Nru I was added to the 5′ end of the HA1 sense primer. Likewise, a recognition site of Sal I and Mlu I was added to the 5′ end of the HA1 antisense primer, a recognition site of Sal I and Acc III was added to the 5′ end of the HA2 sense primer, and a recognition site of Pci I and Nru I was added to the 5′ end of the HA2 antisense primer.

A DNA sequence containing an on sequence, which is a replication origin within E. coli (Escherichia coli), and an ampicillin-resistant gene was cloned by a PCR reaction from DNA of a pQBI25 plasmid vector (Wako Pure Chemical Industries, Ltd.). The following primer sequences were used in the PCR reaction. In the PCR reaction, a recognition site of restriction enzymes Pci I and Sse 8387I was added to the 5′ end and the 3′ end of the sense primer.

Ecoli sense primer:
(SEQ ID No. 27)
5′-ACATGTGAGCAAAAGGCCAGCAAAAGGCCAGGAAC-3′
Ecoli antisense primer:
(SEQ ID No. 28)
5′-CCTGCAGGGACGTCAGGTGGCACTTTTCGGGGAAATGTGC-3′

HA1, HA2, and a DNA sequence containing an on sequence and an ampicillin-resistant gene were cloned by PCR reaction, respectively. These three DNA sequences were cleaved with restriction enzymes Pci I, Sse 8387I, and Sal 1, and subjected to a ligation reaction to obtain a pHA12 plasmid vector. The pHA12 plasmid vector was then cleaved with restriction enzymes Mlu 1 and Acc III to obtain DNA sequence 1 in which HA2, the DNA sequence containing the on sequence and the ampicillin resistance gene, and HA1 were linked in that order from the 5′-end.

Further, a DNA fragment containing a restriction enzyme Bam HI recognition site was obtained from pcDNA3,1-Hygro(+) (Invitrogen). Restriction enzymes Mlu I and Acc III sites were added to the end, followed by PCR amplification to obtain DNA sequence 2. This fragment was linked to DNA sequence 1 to obtain a p23HA12 plasmid vector.

Construction of Basic Vector

The p23HA12 plasmid vector was cleaved with Mlu I and Bam] HI. A DNA fragment obtained by subjecting linker 2-s oligo and linker 2-a oligo to annealing was inserted thereinto to obtain a basic vector II.

Linker 2-s oligo:
(SEQ ID No. 29)
5′-CGCGTatcTCTAGAataATCGATagaAAGCTTacaG-3′
Linker 2-a oligo
(SEQ ID No. 30)
5′- GATCCtgtAAGCTTtctATCGATtatTCTAGAgatA-3′

A DNA sequence 3 containing an SV40 promoter, a poly A signal sequence, and a neomycin-resistant gene (an expression cassette containing SV40, neo R, and synthetic poly A) was amplified by a PCR reaction from DNA of a pQBI15 plasmid vector (Wako Pure Chemical Industries, Ltd.). The following primer sequences were used in the PCR reaction. In the PCR reaction, a recognition site of a restriction enzyme Xba I was added to the 5′ end of the sense primer, and a recognition site of synthetic poly A and Cla I was added to the 3′ end of the sense primer.

neo R sense primer:
(SEQ ID No. 31)
5′-ccttTCTAGActtctgaggcggaaagaacc-3′
neo R antisense primer:
(SEQ ID No. 32)
5′-cttATCGATtCACACAAAAAACCAACACACAGATGTAATGAAAAT
AAAGATATTTTATTgtgggcgaagaactccagca-3′

The basic vector II was cleaved with restriction enzymes Xba I and Cla I, and DNA sequence 3 was ligated thereto to obtain a basic vector+Neo R plasmid.

Construction of Antibody Heavy Chain Expression Cassette

A variable region was amplified by PCR with a primer set of IGH-3A21s and IGH-3A21a1 using a plasmid holding a heavy chain variable region (“Cloning of cDNA and Characterization of Anti-RNase A Monoclonal Antibody 3A21”: Journal of Fermentation and Bioengineering. Vol. 82, No. 3, pp. 312-314, 1999) as a template to obtain DNA sequence 4.

IGH-3A21s primer:
(SEQ ID No. 33)
5′- TAAAAGGTGTCCAGGATGTGCAGTTTCAGG -3′
IGH-3A21a1 primer:
(SEQ ID No. 34)
5′- GAGGCCGATGAAACAGTGACCAGAGTCCCT -3′

RNA extracted from RPMI8226 culture cells with ISOGEN (NIPPON GENE CO., LTD.) was used as a template, and RT-PCR was performed with One-step RT-PCR kit (QIAGEN). A constant region was amplified by PCR with a primer set of IGH-Cs and IGH-Ca using the resultant mRNA as a template to obtain DNA sequence 5.

IGH-Cs primer:
(SEQ ID No. 35)
5′- CATCGGCCTCCACCAAGGGCCCATCGGTCT -3′
IGH-Ca primer:
(SEQ ID No. 36)
5′- TTAAGCGGCCGCTCATTTACCCGGAGACAG -3′

PCR amplification was then performed with a primer set of IGH-LS and IGH-Va using DNA sequence 4 as a template to obtain DNA sequence 6 with a secretion signal added thereto.

IGH-LS primer:
(SEQ ID No. 37)
5′-GGTCGCCACCATGGAGTTTGGACTGAGCTGGGTTTTCCTTGTTGC
TATTTTAAAAGGTGTCCAGGATGTG-3′
IGH-Va primer:
(SEQ ID No. 38)
5′-GCCCTTGGTGGAGGCCGATGAAACAGTGAC -3′

Assemble PCR was performed with a primer set of IGH-Vs and IGH-Ca using DNA sequence 5 and DNA sequence 6 as a template to obtain DNA sequence 7 comprising both the sequences linked to each other.

IGH-Vs primer:
(SEQ ID No. 39)
5′-TTCCGGTACCGGTCGCCACCATGGAGTTTG-3′

A pmaxGFP plasmid (amaxa) was subjected to a PCR reaction for the introduction of mutation with a NotI-site-s/NotI-site-a primer set using Prime STAR MAX (Takara Shuzo Co., Ltd.) to obtain a pmaxGFP+NotI plasmid containing a NotI site introduced just before the CMV promoter.

NotI-site-s primer:
(SEQ ID No. 40)
5′-ATGCggccgcATGTCAATATTGGCCATT -3′
NotI-site-a primer:
(SEQ ID No. 41)
5′-GACATgcggccGCATGGGAGGAGACCGGG -3′

A 0.5-kbp fragment was then amplified with a NotI-spacerA-s/NotI-spacerA-a primer set using pcDNA3.1-hygro(+) (Invitrogen) as a template and was inserted into the NorI site of pmaxGFP+NotI to obtain a pmaxGFP+spacerA plasmid.

NotI-spacerA-s primer:
(SEQ ID No. 42)
5′-ttGCGGCCGCgaaaaagcctgaactcaccg -3′
NotI-spacer A-a primer:
(SEQ ID No. 43)
5′-ttGCGGCCGCgacggtgtcgtccatcacag -3′

A heavy chain was amplified with a KpnI-IGH-s/BglII-IGH-a primer set using DNA sequence 7 as a template, followed by replacement with GFP in pmaxGFP+spacerA to obtain a pmax+spacerA+IgH plasmid.

KpnI-IGH-s primer:
(SEQ ID No. 44)
5′-ccttGGTACCGAAGCCGCTAGCGCTACCGGTCGCCACCaTGGAGT
TTGGACTGAGCTGGG-3′
BgIII-IGH-a primer:
(SEQ ID No. 45)
5′-ccttAGATCTtcatttacccggagacaggg-3′

PCR amplification was performed with a MluI-spA(IgH)-s2/MluI-CMV-IgHL-SVpA-a primer set using pmax+spacerA+IgH as a template. The amplification product was cloned into a pCR-BluntII-TOPO vector (Invitrogen), followed by cleaving with a restriction enzyme Mlu I to obtain DNA sequence 8 (heavy chain cassette).

MluI-spA(IgH)-s2 primer:
(SEQ ID No. 46)
5′-ccttACGCGTgacggtgtcgtccatcacag-3′
MluI-CMV-IgHL-SVpA-a primer:
(SEQ ID No. 47)
5′-cttACGCGTAACGTCTCGCCCTTTGGTCTC-3′

Construction of Antibody Light Chain Expression Cassette

Likewise, a plasmid holding a light chain variable region was provided as a template, and the variable region was amplified by PCR with a primer set of IGL-3A21s1 and IGL-3A21a1 to obtain DNA sequence 9.

IGL-3A21s1 primer:
(SEQ ID No. 48)
5′-CCCAGGTGCCAGATGTGACATCAAGATGAC-3′
IGL-3A21a1 primer:
(SEQ ID No. 49)
5′-GCCACAGTTCGTTTTATTTCCAACTTTGTC-3′

RNA extracted from RPMI8226 culture cells with ISOGEN was provided as a template, and RT-PCR was performed with One-step RT-PCR kit. A constant region was amplified by PCR with a primer set of IGL-Cs and IGL-Ca using the resultant mRNA as a template to obtain DNA sequence 10.

IGL-Cs primer:
(SEQ ID No. 50)
5′-TGGAAATAAAACGAACTGTGGCTGCACCAT-3′
IGL-Ca primer:
(SEQ ID No. 51)
5′-TTAAGCGGCCGCCTAACACTCTCCCCTGT-3′

PCR amplification was performed with a primer set of IGL-LS and IGL-3A21a using DNA sequence 9 as a template to obtain DNA sequence 11 with a secretion signal added thereto.

IGL-LS primer:
(SEQ ID No. 52)
5′-GGTCGCCACCATGGACATGAGGGTCCCCGCTCAGCTCCTGGGGCTC
CTGCTGCTCTGGCTCCCAGGTGCCAGATGTGACA-3′
IGL-3A21a primer:
(SEQ ID No. 53)
5′-GCCACAGTTCGTTTTATTTCCAACTTTGTC-3′

Assemble PCR was performed with a primer set of IGL-Vs and IGL-Ca using DNA sequence 10 and DNA sequence 11 as a template to obtain DNA sequence 12 comprising both products linked to each other.

IGL-Vs primer:
(SEQ ID No. 54)
5′-CCTTGGTACCGGTCGCCACCATGGACATGA-3′

A 0.5-kbp fragment was then amplified with a NotI-spacerB-s/NotI-spacerB-a primer set using pcDNA3.1-hygro(+) (Invitrogen) as a template and was inserted into the NorI site of pmaxGFP+NotI to obtain a pmaxGFP+spacerB plasmid.

NotI-spacerB-s primer:
(SEQ ID No. 55)
5′-ttGCGGCCGCctgtgatggacgacaccgtc-3′
NotI-spacerB-a primer:
(SEQ ID No. 56)
5′-ttGCGGCCGCctattcctttgccctcggac-3′

A light chain was then amplified with a KpnI-IGH-s/BglII-IGH-a primer set using DNA sequence 12 as a template, followed by replacement with GFP in pmaxGFP+spacerB to obtain a pmax+spacerB+IgL plasmid.

KpnI-IGL-s primer:
(SEQ ID No. 57)
5′-ccttGGTACCGAAGCCGCTAGCGCTACCGGTCGCCACCatggaca
tgagggtccccgc-3′
BgIII-IGL-a primer:
(SEQ ID No. 58)
5′-ccttAGATCTctaacactctcccctgttga -3′

PCR amplification was performed with a HindIII-spB(IgL)-s2/Hin3-CMV-IgHL-SVpA-a primer set using pmax+spacerB+IgL as a template. The amplification product was cloned into a pCR-BluntII-TOPO vector, followed by cleaving with a restriction enzyme Hind III to obtain DNA sequence 13 (light chain cassette).

HindIII-spB(IgL)-s2 primer:
(SEQ ID No. 59)
5′-ccttAAGCTTctattcctttgccctcggac-3′
Hin3-CMV-IgHL-SVpA-a primer:
(SEQ ID No. 60)
5′-cttAAGCTTAACGTCTCGCCCTTTGGTCTC-3′

A basic vector+neo R plasmid was cleaved with a restriction enzyme Hind III and was inserted into DNA sequence 13 (light chain cassette) to obtain a basic vector+neoR+IgL plasmid.

Acquisition of DNA Fragment Homologous to hprt Gene of CHO Cells

Genome DNA of CHO-K1 cells was provided as a template, and DNA fragments (CHA1 and CHA2) homologous to an hprt gene as a target were cloned by a PCR reaction (KOD-Plus-Ver2, TOYOBO). As shown in FIG. 3 which will be described later, CHA1 and CHA2 were set to sequences homologous to a region including exon 3 in the hprt gene. The following primer sequences were used in the PCR reaction.

SseNru-CHA1-1k-s primer:
(SEQ ID No. 61)
5′-ttCCTGCAGGTCGCGAaggagtttattagaggaaatat-3′
MluI-CHA1-r primer:
(SEQ ID No. 62)
5′-ttACGCGTtgataaaatctacagtcatggg-3′
Bam-CHA2-s primer:
(SEQ ID No. 63)
5′-ttGGATCCgactgaagagctactgtgta-3′
PciNru-CHA2-1k-r primer:
(SEQ ID No. 64)
5′-ttACATGTTCGCGAatcagatccctgggactgga-3′

Acquisition of CHO 1k×2 Antibody Vector

A CHA2 sequence amplified with a Bam-CHA2-s/PciNru-CHA2-1k-r primer set was cleaved with restriction enzymes Bam HI and Pci I, followed by replacement with the HA2 sequence in the basic vector+neoR+IgL plasmid. Further, a CHA1 sequence amplified with an SseNru-CHA1-1k-s/MluI-CHA1-r primer set was cleaved with restriction enzymes Sse8387I and Mlu I, followed by replacement with the HA1 sequence. Next, DNA sequence 8 (heavy chain cassette) was inserted into the restriction enzyme Mlu I site to obtain a CHO 1k×2 antibody vector shown in FIG. 2 (A).

Acquisition of HT1080 Homology Arm Vector

DNA sequence 8 (heavy chain cassette) was inserted into a restriction enzyme Mlu I site in the basic vector+neoR+IgL plasmid to obtain an HT1080 homology arm vector shown in FIG. 2 (B).

### Example 3

### Introduction of Vector into Cells

Linearization of Vector

The CHO 1k×2 antibody vector and the HT1080 homology arm vector were purified with Endofree Plasmid Maxi kit (QIAGEN) and were cleaved with Nru I, followed by dissolution in sterilized water to a concentration of 2 g/L, and the solutions were used in the following transfection experiment.

Transfection and Screening

CHO-K1 cells were adjusted with a nucleofection T solution (amaxa) to 1×10⁶ cells and were mixed with 2 μg of a linearized plasmid vector. Next, the mixed solution thus obtained and Nuceofector II (amaxa) were used to apply voltage with program U-023. The transfection was performed three times for each vector. The transfected cells were seeded in a 96 well plate at 350 cells/well and cultured in an incubator under conditions of 37° C. and 5% CO₂ (medium: Advanced MEM (GIBCO) supplemented with 5% FBS, 1× Glutamax (GIBCO)), and, 2 days after the transfection, G418 (Invitrogen) was added (final concentration; 500 μg/mL).

After 4 days from the transfection, G418 (final concentration; 500 μg/mL) and 6-TG (final concentration; 50 μM) (Wako Pure Chemical Industries, Ltd.) were added, followed by culture for 6 days. After the culture, all the wells were confirmed, and G418/6TG-resistant colonies were isolated. The results were as shown in Table 1.

The results were as shown in Table 1. The number of G418/6TG-resistant clones obtained by the CHO 1k×2 antibody vector was 6 clones per 3×10⁶ cells. On the other hand, in the HT1080 homology arm vector, G418/6TG-resistant clones were not obtained.

TABLE 1

| | | Number of | Number of |
|---|---|---|---|
| | Number of | integrated | integrated |
| | G418/6TG- | clones/number of | clones/number of |
| | resistant | clones as | clones as |
| | clones | determined by | determined by |
| Introduced | acquired (per | genome PCR | Southern |
| vector | 3 × 10⁶ cells) | analysis | hybridization analysis |
| CHO 1 kb × | 6 | 5/6 | 5/5 |
| 2 antibody | | | |
| vector | | | |
| HT1080 | 0 | NOT TEST | NOT TEST |
| homology | | | |
| arm vector | | | |

### Example 4

### Analysis by Genome PCR

Genome DNA was extracted with DNA Isolation kit (Roche) from clones that were obtained by the transfection of the CHO 1k×2 antibody vector and were resistant to G418 and 6TG. Recombination regiospecific to target hprt locus was confirmed by the following PCR reaction using the genome DNA as a template.

FIG. 3 (A) is a typical diagram for explaining details of an analysis of a homologous recombination reaction by genome PCR.

A target region (2) in homologous recombination of hprt gene (1) is set so as to contain exon 3 (3). CHA1 (4), CHA2 (5) and an antibody heavy chain sequence (7), a neomycin-resistant gene (8), and an antibody light chain sequence (9) held between CHA1 (4) and CHA2 (5) in the vector DNA (6) are integrated into this region by homologous recombination.

DNA (1688 bp DNA) containing CHA1 (4) and a part of the antibody heavy chain sequence (7) and DNA (1752 bp DNA) containing CHA2 (5) and a part of the antibody light chain sequence (9) can be acquired from the genome only by homologous recombination and thus can be an index for homologous recombination.

Accordingly, a 1688 bp DNA shown in FIG. 3 (A) was set as an index for homologous recombination between CHA1 and the target region, and the DNA was detected by a PCR reaction with primer CHPRTs/NotI-spacerA-s as shown below. On the other hand, 1752 bp DNA shown in FIG. 3 (A) was set as an index for homologous recombination between CHA2 and the target hprt locus, and the DNA was detected by a PCR reaction with primer IGL-Cs/CHA2-seq-a1 as shown below.

CHPRTs primer:
(SEQ ID No. 65)
5′-TGTTCCTGTGCATACTAGGC-3′
CHA2-seq-a1 primer:
(SEQ ID No. 66)
5′-CCAGAGAAATTATTTGCCACCAGC-3′

Next, the PCR amplification products thus obtained were analyzed by electrophoresis on 1.0% agarose gel. The results were as shown in FIGS. 3 (B) and (C).

In FIGS. 3 (B) and (C), numbers 1 to 6 represent clones obtained with the CHO 1k×2 antibody vector. Both the 1688 bp and 1752 bp PCR amplification products were confirmed in 5 clones among the acquired 6 clones, indicating that a homologous recombination reaction took place.

### Example 5

### Analysis by Southern Hybridization

The integration of the CHO 1k×2 antibody vector into the target hprt locus by regiospecific recombination in 5 clones acquired in Example 4 was confirmed by the following Southern hybridization.

FIG. 4 (A) is a typical diagram for explaining details of Southern hybridization in Example 5.

As shown in FIG. 4 (A), a target region (2) in homologous recombination of hprt gene (1) is set so as to contain exon 3 (3), and homology arm 1 (CHA1) (4) and homology arm 2 (CHA2) (5) are set so as to be homologous to two adjacent regions in the target region. Pci I restriction enzyme sites are located so as to hold the target region (2) therebetween and are absent in the target region (2). On the other side, the Pci I restriction enzyme site is absent in the CHO 1k×2 antibody vector. A neomycin-resistant gene sequence (6) in the CHO 1k×2 antibody vector was designed so that an NR probe can be hybridized.

As shown in FIG. 4 (A), when 10488 bp DNA has been detected, it is determined, from the position of the Pci I restriction enzyme sites and the chain length of the DNA sequence, that the CHO 1k×2 antibody vector has been integrated into the hprt locus of the clone.

Manufacturing of NR Probe

An NR probe having a sequence complementary to a neomycin-resistant gene in the CHO 1k×2 antibody vector was synthesized according to the following procedure. At the outset, the entire length of a neomycin-resistant gene coding sequence in the CHO 1k×2 antibody vector was amplified by PCR, followed by TA cloning into a pGEM T plasmid vector (Promega). Next, a DIG (Digoxigein) labeled probe was prepared with a PCR DIG probe synthesis kit (Roche, primer: M13 Forward/Reverse primer).

Preparation of Membrane

Genome DNA of each cell clone was extracted from the 6TG resistant colony and was cleaved with a Pci I restriction enzyme. The cleaved genome DNA (5 μg) was electrophoresed on 0.6% agarose gel and blotted onto a nylon membrane (Hybond N+ membrane, Amaersham Biosciences). The membrane thus obtained was incubated at 80° C. for 2 hr to immobilize DNA on the membrane.

Hybridization

The membrane was hybridized with the NR probe. In this case, prehybridization, hybridization and probe detection were carried out according to a DIG Application Manual (Roche).

As shown in FIG. 4 (B), in all of the analyzed 5 clones, a 10488 bp DNA fragment was detected by the NR probe.

### Example 6

### Production of Protein by Recombinant Cells

Culture of Recombinant Clone and Sampling of Medium

At the outset, the recombinant clone was seeded in an amount of 2×10⁵ cells per plate and was cultured in 10 mL of Advanced MEM (Invitrogen) containing G418 (final concentration; 500 μg/mL), 6-TG (final concentration; 50 μM), and FBS (Japan Bio Serum) (5%) in the presence of 5% CO₂ at 37° C. On the fifth day from the start of the culture, the medium was collected. The collected medium was used for the following ELISA analysis.

Quantitative Determination by ELISA

The amount of IgG in the collected medium was determined by measuring the absorbance at 450 nm with Human IgG EIA Kit (precoated) (Takara Shuzo Co., Ltd.).

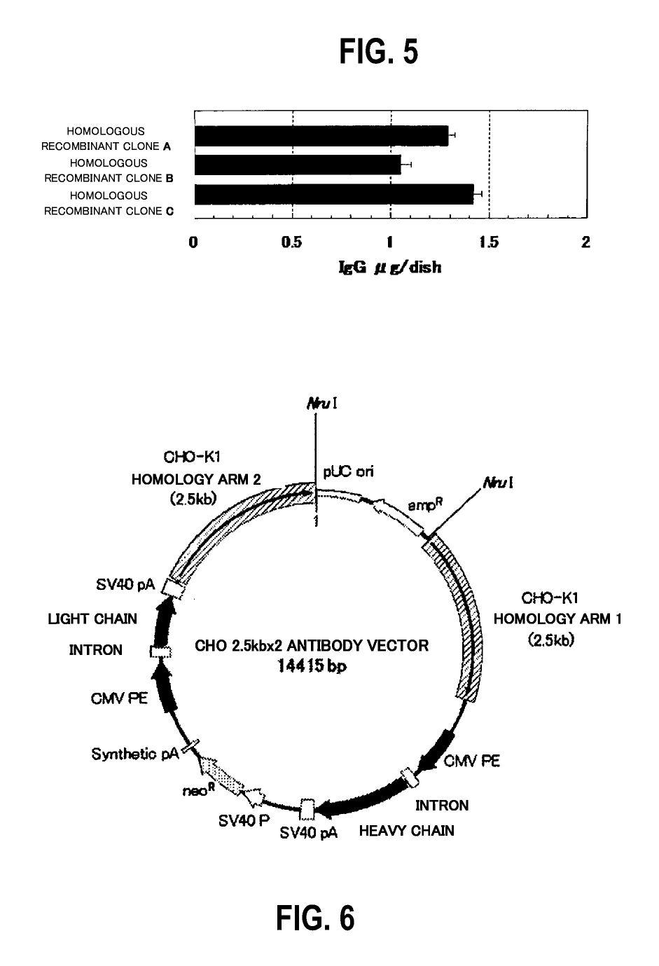

The results were as shown in FIG. 5. In the measured 3 clones, the amount of IgG per plate was 1.29±0.18 μg/dish on average, and the CV value was 14.2%.

### Example 7

### Homologous Recombination with Vector Having Homologous DNA Fragment Having Chain Length of 2.5 kbp

Acquisition of 2.5-kbp DNA Fragment Homologous to hprt Gene in CHO Cells

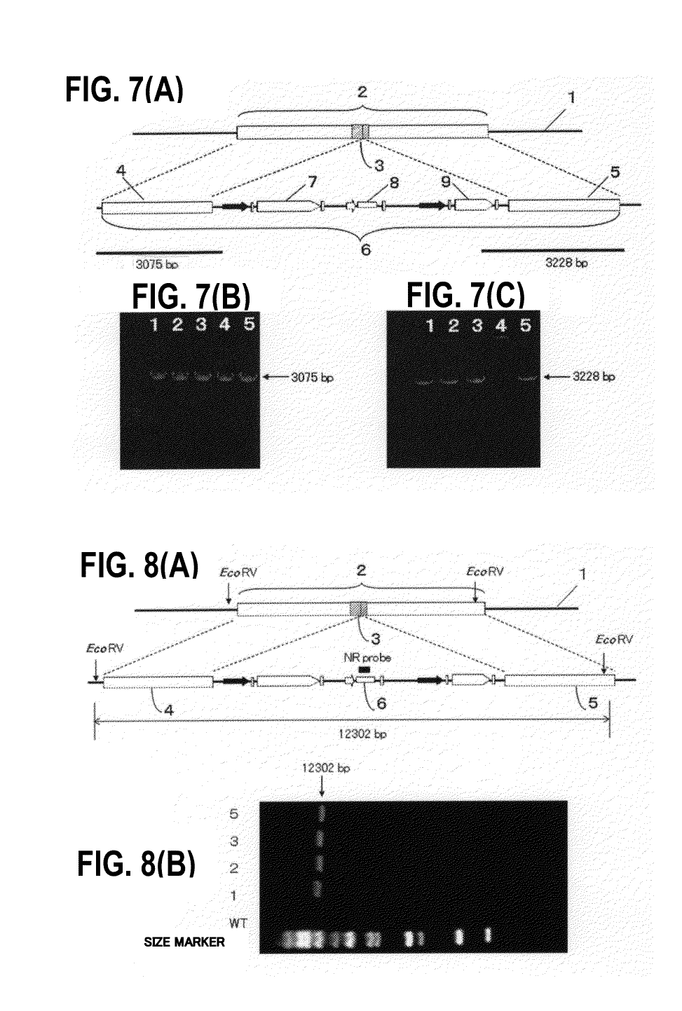

In order to manufacture a CHO 2.5 k×2 antibody vector that has a homologous DNA fragment having a chain length of 2.5 kbp and is shown in FIG. 6, DNA fragments homologus to target hprt gene (CHA1-2.5 and CHA2-2.5) were cloned by a PCR reaction (KOD-Plus-Ver2, TOYOBO) using genome DNA of CHO-K1 cells as a template. As shown in FIG. 7 (A) which will be described later, CHA1-2.5 and CHA2-2.5 were set to a sequence homologous to a region containing exon 3 of the hprt gene. MluI-CHA1-r primer and Bam-CHA2-s primer described in Example 2 and the following primers were used as primer sequences in the PCR reaction.

SseNru-CHA1-2.5k-s primer:
(SEQ ID No. 67)
5′-ttCCTGCAGGTCGCGAgtctgtgtgtatgtttgtgataggc-3′
NcoNru-CHA2-2.5k-r primer:
(SEQ ID No. 68)
5′-ttCCATGGTCGCGAtgaaggttatagagcataggggacc-3′

Acquisition of CHO 2.5 k×2 Antibody Vector

The CHA2-2.5 sequence amplified with the Bam-CHA2-s/NcoNru-CHA2-2.5 k-r primer set was cleaved with restriction enzymes Bam HI and Nco I, followed by replacement with the HA2 sequence in the basic vector+neo R+IgL plasmid. Further, the CHA1-2.5 sequence amplified with the SseNru-CHA1-2.5 k-s/MluI-CHA1-r primer set was cleaved with restriction enzymes Sse8387I and Mlu I, followed by replacement with the HA1 sequence. Next, DNA sequence 8 (heavy chain cassette) was inserted into the restriction enzyme Mlu I site to obtain a CHO 2.5 k×2 antibody vector shown in FIG. 6.

Acquisition of CHO 2.5 k×2 Antibody Vector Integrated Clone

The CHO 2.5 k×2 antibody vector was purified and linearized in the same manner as in Example 3. The linearized vector was dissolved in sterilized water to a concentration of 2 g/L, followed by introduction into 1×10⁶ cells.

Next, screening with a selective drug was carried out in the same manner as in Example 3. As a result, G418/6TG-resistant colonies of 5 clones were isolated.

Analysis by Genome PCR

Genome DNA was extracted from clones resistant to G418 and 6TG obtained by the transfection of the CHO 2.5 k×2 antibody vector, and recombination regiospecific to the target hprt locus was confirmed by the following PCR reaction using the genome DNA as a template.

FIG. 7 (A) is a typical diagram for explaining details of an analysis of a homologous recombination reaction by genome PCR.

A target region (2) of homologous recombination in an hprt gene (1) is set so as to contain exon 3 (3). CHA1-2.5 (4), CHA2-2.5 (5), an antibody heavy chain sequence (7), a neomycin-resistant gene (8), and an antibody light chain sequence (9) held between CHA1-2.5 (4) and CHA2-2.5 (5) in a vector DNA (6) are integrated into this region by homologous recombination.

DNA (3075-bp DNA) containing CHA1-2.5 (4) and a part of the antibody heavy chain sequence (7) and DNA (3228-bp DNA) containing CHA2-2.5 (5) and a part of the antibody light chain sequence (9) can be acquired from the genome only by homologous recombination and thus can be an index for homologous recombination.

Accordingly, a 3075-bp DNA shown in FIG. 7 (A) was set as an index for homologous recombination between CHA1-2.5 and the target region, and the DNA was detected by a PCR reaction with primer CHA1-seq-s11/NotI-spacerA-s. On the other hand, 3228-bp DNA shown in FIG. 7 (A) was set as an index for homologous recombination between CHA2-2.5 and the target hprt locus, and the DNA was detected by a PCR reaction with primer IGL-Cs/CHA2-seq-a4.

CHA1-seq-s11 primer:
(SEQ ID No. 69)
5′-GACACATGCAGACAGAACAG-3′
CHA2-seq-a4 primer:
(SEQ ID No. 70)
5′-GTTTGCTAACACCCCTTCTC-3′

Next, the PCR amplification products thus obtained were analyzed by electrophoresis on 1.0% agarose gel. The results were as shown in FIGS. 7 (B) and (C).

In FIGS. 7 (B) and (C), numbers 1 to 5 represent clones obtained with the CHO 2.5 k×2 antibody vector. Both the 3075-bp and 3228-bp PCR amplification products were confirmed in 4 clones among the acquired 5 clones, indicating that a homologous recombination reaction took place.

Analysis by Southern Hybridization

The regiospecific integration of the CHO 2.5 k×2 antibody vector into the target hprt locus in 4 clones acquired in Example 7 was confirmed by the following Southern hybridization.

FIG. 8 (A) is a typical diagram for explaining details of Southern hybridization in Example 7

As shown in FIG. 8 (A), a target region (2) in homologous recombination of hprt gene (1) is set so as to contain exon 3 (3), and homology arm 1 (CHA1-2.5) (4) and homology arm 2 (CHA2-2.5) (5) are set so as to be homologous to two adjacent regions in the target region. Only one Eco RV restriction enzyme site is present around the target region (2), and only one Eco RV restriction enzyme site is present within the target region. On the other hand, in the CHO 2.5 k×2 antibody vector, only one Eco RV restriction enzyme site is present in CHA2-2.5. A neomycin-resistant gene sequence (6) in the CHO 2.5 k×2 antibody vector was designed so that an NR probe can be hybridized.

As shown in FIG. 8 (A), when 12302-bp DNA has been detected, it is determined, from the position of the Eco RV restriction enzyme sites and the chain length of the DNA sequence, that the CHO 2.5 k×2 antibody vector has been integrated into the hprt locus of the clone.

Manufacturing of Probe

An NR probe having a sequence complementary to a neomycin-resistant gene in the CHO 2.5 k×2 antibody vector was provided in the same manner as in Example 5.

Preparation of Membrane

Genome DNA of each cell clone was extracted from the 6TG resistant colony and was cleaved with an Eco RV restriction enzyme. The cleaved genome DNA was subjected to a Southern hybridization analysis in the same manner as in Example 5.

As shown in FIG. 8 (B), in all of the analyzed 4 clones, a 12302-bp DNA fragment was detected by the NR probe.

### Example 8

### Confirmation of Homologous Recombination in Two Chromosomes of CHO Cells

Confirmation of Remained Wild-Type hprt Gene by Genome PCR

Whether or not a wild-type hprt gene remains was confirmed by the following PCR reaction using genome DNA extracted from homologous recombination clones of the CHO 1k×2 antibody vector and the CHO 2.5 k×2 antibody vector as a template.

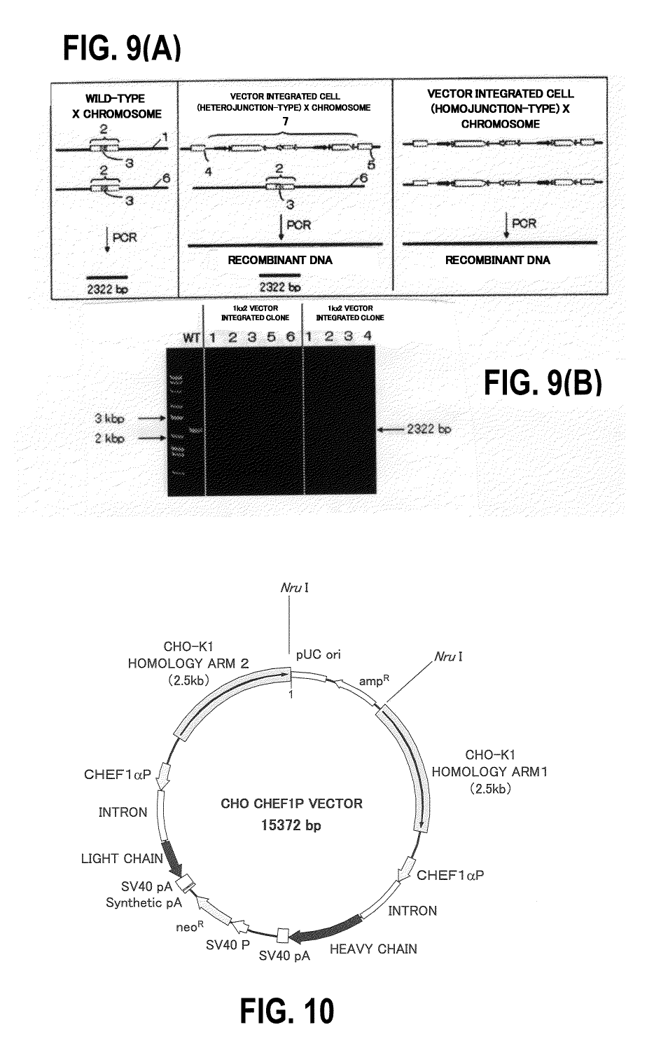

FIG. 9 (A) is a typical diagram for explaining details of genome PCR for the confirmation of remained wild-type hprt gene.

At the outset, the left drawing in FIG. 9 (A) shows PCR in wild-type CHO cells having two X chromosomes. A target region (2) in homologous recombination of an hprt gene (1) is set so as to contain exon 3 (3). In vector non-introduced wild-type cells, DNA (2322 bp) of the target region can be amplified by a PCR reaction, and, thus, the remaining of the wild-type hprt gene can be confirmed using this as an index.

The drawing located at the center of FIG. 9 (A) shows PCR in heterojunction-type homologous recombinant cells comprising a vector integrated into an hprt locus of one X chromosome. In one chromosome, vector DNA (7) (containing CHA1(4), CHA2(5), and an antibody heavy chain, a neomycin-resistant gene, and an antibody light chain sequence held between CHA1(4) and CHA2(5)) is integrated into the target region (2) by homologous recombination. On the other hand, another chromosome holds a target region, and DNA (2322 bp) in the target region can be amplified by a PCR reaction, making it possible to confirm the remaining of the wild-type hprt gene.

On the other hand, as shown in the drawing located right in FIG. 9 (A), in homojunction-type homologous recombinant cells comprising a vector integrated into both X chromosomes, only recombinant DNA is expressed, and DNA (2322 bp) in the target region is not amplified by the PCR reaction.

Based on the foregoing understanding, the 2322-bp DNA shown in FIG. 9 (A) was set as the index, and, for the DNA detection, a PCR reaction was carried out with primer CHPRTs/CHA2-seq-a1.

Next, the PCR amplification product thus obtained was analyzed by electrophoresis on 1.0% agarose gel. The results were as shown in FIG. 9 (B).

In FIG. 9 (B), WT represents wild-type cells, 1k×2 vector integrated clones (1, 2, 3, 5, and 6) represent CHO 1k×2 antibody vector integrated clones acquired in Example 4, and 2.5 k×2 vector integrated clones (1 to 4) represent CHO 2.5 k×2 antibody vector integrated clones acquired in Example 7. 2322-bp PCR amplification products were confirmed from the wild-type cells, and, thus, the presence of the wild-type hprt gene was confirmed. On the other hand, in all the analyzed recombinant clones, any 2322-bp PCR amplification product was not confirmed, indicating that the recombinant cells were of a homojunction type in which a vector was integrated into both X chromosomes.

From the results, it was confirmed that the use of the vector according to the present invention allowed recombinant clones comprising a target protein gene integrated into both hprt locuses of two X chromosomes to be acquired with high frequency.

### Example 9

### Long-Term Stable Expression of Antibody by Recombinant Vector Clones Using CHO Endogenous Promoter

Manufacturing of CHO CHEF1P Vector

A CHEF1α promoter was amplified with an EF1aP-MluI-F/EF1aP-XbaI-R primer set using a CHO-K1 genome DNA as a template.

EF1aP-MluI-F primer:
(SEQ ID No. 71)
5′-AGAACGCGTCCACACAATCAGAACCACA-3′
EF1aP-XbaI-R primer:
(SEQ ID No. 72)
5′-GACGATCTAGAGGTGGTTTTCACAACA-3′

Further, an IgH gene was amplified with a CMV-seq-s/MluI-CMV-IgHL-SVpA-a primer set using a CHO 2.5 k×2_H-neo-L plasmid as a template.

CMV-seq-s primer:
(SEQ ID No. 73)
5′-AGGGACTTTCCATTGACGTC-3′
MluI-CMV-IgHL-SVpA-a primer:
(SEQ ID No. 74)
5′-cttACGCGTAACGTCTCGCCCTTTGGTCTC-3′

Next, the CHEF1αP and IgH gene thus obtained were cleaved with XbaI and NheI, respectively, followed by ligation. Thereafter, a band of the CHEF1αP+IgH gene was subjected to gel extraction, and the CHEF1αP+IgH gene was cloned into a TOPO vector (Invitrogen), followed by cleaving with MluI to obtain a CHEF1αP+IgH gene cassette.

On the other hand, a CHEF1α promoter was amplified with an EF1aP-HindIII-F/EF1aP-HindIII-R primer set using CHO-K1 genome DNA as a template.

EF1aP-HindIII-F primer:
(SEQ ID No. 75)
5′-AGAAAGCTTCCACACAATCAGAACCACA-3′
EF1aP-HindIII-R primer:
(SEQ ID No. 76)
5′-GACGAATTCGCGGTGGTTTTCACAACA-3′

An IgL gene was amplified with an IgLser-EcoRI-F/IgLser-HindIII-R primer set using a CHO 2.5 k×2_H-neo-L plasmid as a template.

IgLser-EcoRI-F primer:
(SEQ ID No. 77)
5′-TATGAATTCGTCGCCACCATGGACAT-3′
IgLser-HindIII-R primer:
(SEQ ID No. 78)
5′-TGTAAGCTTTACCACATTTGTAGAGGTTTT-3′

The CHEF1αP and IgL gene thus obtained were cleaved with EcoRI, followed by ligation. Thereafter, a band of the CHEF1αP+IgL gene was subjected to gel extraction, and the CHEF1αP+IgL gene was cloned into a TOPO vector, followed by cleaving with HindIII to obtain a CHEF1αP+IgL gene cassette.

The CHO 2.5 k×2_H-neo vector was cleaved with HindIII and was dephosphorylated, and the CHEF1αP+IgL gene cassette was inserted thereinto to obtain an H-neo-EF1L vector. Next, after MluI cleaving and dephosphorylation, the CHEF1αP+IgH gene cassette was inserted thereinto to obtain a CHO CHEF1P vector shown in FIG. 10.

Acquisition of CHO CHEF1P Vector Recombinant CHO Cells

Linearization, introduction into CHO cells, and screening of the constructed CHO CHEF1P vector were carried out in the same manner as in Example 3 to acquire CHO CHEF1P vector recombinant CHO cells.

Confirmation of Long-Term Stability in Antibody Production of CHO CHEF1P Vector Clone

The acquired CHO CHEF1P vector recombinant CHO cells were subcultured (selective drug-free medium: Advanced MEM (GIBCO) supplemented with 5% FBS, 1× Glutamax (GIBCO)) under conditions of 7° C. and 5% CO₂ and were used in a timely manner for the measurement of the amount of antibody production.

Cells during subculture were peeled off, were seeded at 2×10⁶ cells in a 100-mm dish, and were cultured under conditions of 37° C. and 5% CO₂ overnight. The cultured cells were peeled off and adjusted to 3×10⁵ cells/ml. The cells were seeded in a 12-well plate in an amount of 1.5 ml per well and were cultured. After the elapse of 24 hr from the start of the culture, the medium was entirely removed. A fresh medium (1.5 ml) was dispensed, followed by culture for 24 hr. The whole quantity of the medium was collected, and the amount of IgG was measured by ELISA assay. Cells adhered on the bottom of the wells were peeled off, and the number of cells was counted.

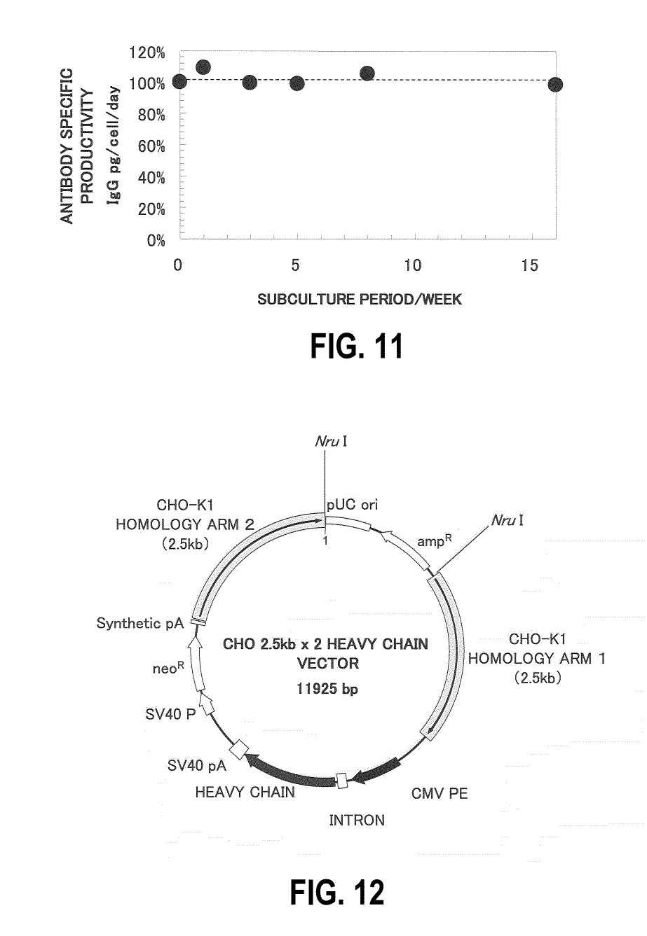

The results were as shown in FIG. 11. The antibody productivity of CHO cells comprising a CHO CHEF1P vector integrated into an hprt locus was kept constant even after subculture for 16 weeks under selective drug-free conditions, and the productivity was not lowered.

### Example 10

### Acquisition of Homologous Recombinant by Vector that has Homologous DNA Fragment Having Chain Length of 2.5 kbp and Expresses Antibody Heavy Chain

Manufacturing of Recombinant Vector that Expresses Antibody Heavy Chain

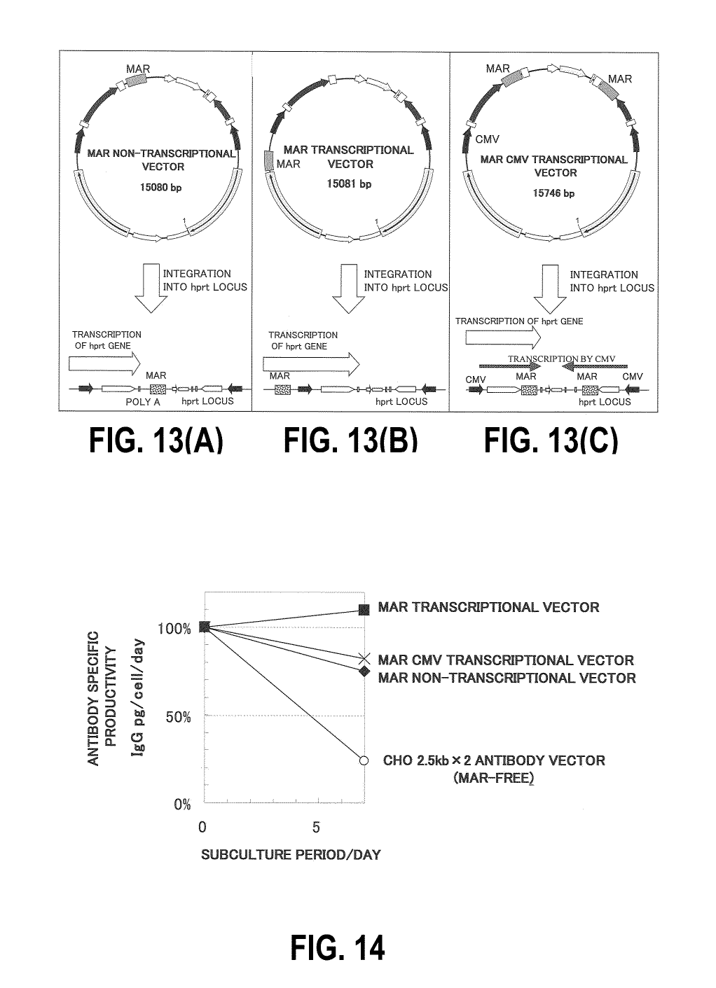

The CHO 2.5 k×2 antibody vector prepared in Example 7 was cleaved with a restriction enzyme Hind III and was subjected to slef-ring closing to prepare a CHO 2.5 kb×2 heavy chain vector shown in FIG. 13.

Acquisition of CHO 2.5 kb×2 Heavy Chain Vector Recombinant CHO Cells

Linearlization, introduction into CHO cells, and screening of the constructed CHO 2.5 kb×2 heavy chain vector were carried out in the same manner as in Example 3 to acquire CHO 2.5 kb×2 heavy chain vector recombinant CHO cells. As a result, 130 recombinant CHO cells per 10⁷ cells were acquired.

### Example 11

### Acquisition of Stable Expression CHO Cells by Vector that has MAR Derived from Human hprt Locus Intron and Expresses Antibody Heavy Chain

Manufacturing of MAR Non-Transcriptional Vector

MAR (hprtMAR1) of a human hprt gene intron was amplified with a XbaI-hprtMAR-s/XbaI-hprtMAR-a primer set using the genome DNA of an HT1080 cell line, a human-derived cell line, provided in Example 2 as a template.

XbaI-hprtMAR-s primer:
(SEQ ID No. 79)
5′-ttTCTAGAtagttatgagcccatgtccc-3′
XbaI-hprtMAR-a primer:
(SEQ ID No. 80)
5′-ttTCTAGAcggtgaaatcctgtctctac-3′

The CHO 2.5 k×2 antibody vector prepared in Example 7 was cleaved with an restriction enzyme XbaI, and hprtMAR1 was inserted thereinto to obtain an MAR non-transcriptional vector shown in FIG. 13 (A).

Manufacturing of MAR Transcriptional Vector

MAR (hprtMAR2) of a human hprt gene intron was amplified with an Ascl-hprtMAR-s/MluI-hprtMAR-a primer set using genome DNA of an HT1080, a human-derived cell line, as a template.

AscI-hprtMAR-s primer:
(SEQ ID No. 81)
5′-ttGGCGCGCCtagttatgagcccatgtccc-3′
MluI-hprtMAR-a primer:
(SEQ ID No. 82)
5′-ttACGCGTcggtgaaatcctgtctctac-3′

The CHO 2.5 k×2 antibody vector prepared in Example 7 was cleaved with a restriction enzyme MluI, and hprtMAR2 was inserted thereinto. Next, the product was cleaved with a restriction enzyme MluI, and the antibody heavy chain expression cassette of Example 2 was inserted thereinto to obtain an MAR transcriptional vector shown in FIG. 13 (B).

Manufacturing of MAR CMV Transcriptional Vector

MAR (hprtMAR3) of a human hprt gene intron was amplified with a BglII-hprtMAR-s/BglII-hprtMAR-a primer set using genome DNA of an HT1080, a human-derived cell line, as a template.

BglII-hprtMAR-s primer:
(SEQ ID No. 83)
5′-ttAGATCTtagttatgagcccatgtccc-3′
BglII-hprtMAR-a primer:
(SEQ ID No. 84)
5′-ttAGATCTcggtgaaatcctgtctctac-3′

The pCR-BluntII-TOPO+antibody heavy chain expression cassette prepared in Example 2 was cleaved with a restriction enzyme BglII, and hprtMAR3 was inserted thereinto. The product was taken off with a restriction enzyme MluI to obtain an antibody heavy chain +MAR expression cassette.

The pCR-BluntII-TOPO+antibody light chain expression cassette prepared in Example 2 was cleaved with a restriction enzyme BglII, and hprtMAR3 was inserted thereinto. The product was taken off with a restriction enzyme HindIII to obtain an antibody light chain +MAR expression cassette.

The CHO 2.5 k×2 antibody vector prepared in Example 7 was cleaved with a restriction enzyme MluI, and an antibody heavy chain +MAR expression cassette was inserted thereinto. Next, the product was cleaved with a restriction enzyme HindIII, and an antibody light chain +MAR expression cassette was inserted thereinto to obtain an MAR CMV transcriptional vector shown in FIG. 13 (C).

Acquisition of MAR Vector Recombinant CHO Cells

Linearization, introduction into CHO cells, and screening of the constructed MAR non-transcriptional vector, MAR transcriptional vector and MAR CMV transcriptional vector were carried out in the same manner as in Example 3 to obtain recombinant CHO cells.

Confirmation of Stability in Antibody Production of Vector Clones that have MAR Derived from Human hprt Locus Intron and Expresses Antibody Heavy Chain

The analysis of antibody productivity of recombinant CHO clones acquired by MAR vector shown in FIGS. 13 (A), (B), and (C) was carried out in the same manner as in Example 9.

The results were as shown in FIG. 14. As is apparent from the results, clones that could maintain antibody productivity were only clones with a vector into which MAR of human hprt is transcribed only by transcription of the hprt gene at the vector integration site being integrated thereinto. For clones acquired by a vector having a human hprtMAR non-transcriptional structure and a vector having a structure that the target protein gene and MAR are simultaneously transcribed by a CMV promoter, the antibody productivity was lowered during culture for 7 days.

### Example 12

### Acquisition of Homologous Recombinant by Vectors that have Homologous DNA Fragments having Chain Lengths of 200b and 500b and Express Antibody Heavy Chain

Manufacturing of CHO 200b×2 Vector and CHO 500b×2 Vector

Homologous DNA fragments (CHA1-200 and CHA2-200) of target hprt gene were cloned by a PCR reaction respectively with an SseNru-CHA1-200-s/MluI-CHA1-r primer set and a Bam-CHA2-s/PciNru-CHA2-200-r primer set using genome DNA of CHO-K1 cells as a template. CHA1-200 and CHA2-200 were set to a sequence homologous to a region containing exon 3 of an hprt gene. The following primer sequences were used in the PCR reaction.

SseNru-CHA1-200-s primer:
(SEQ ID No. 85)
5′-ttCCTGCAGGTCGCGAtggaatcttctattcctgattt-3′
PciNru-CHA2-200-r primer:
(SEQ ID No. 86)
5′-ttACATGTTCGCGAtcagcactcaggagtcagag-3′

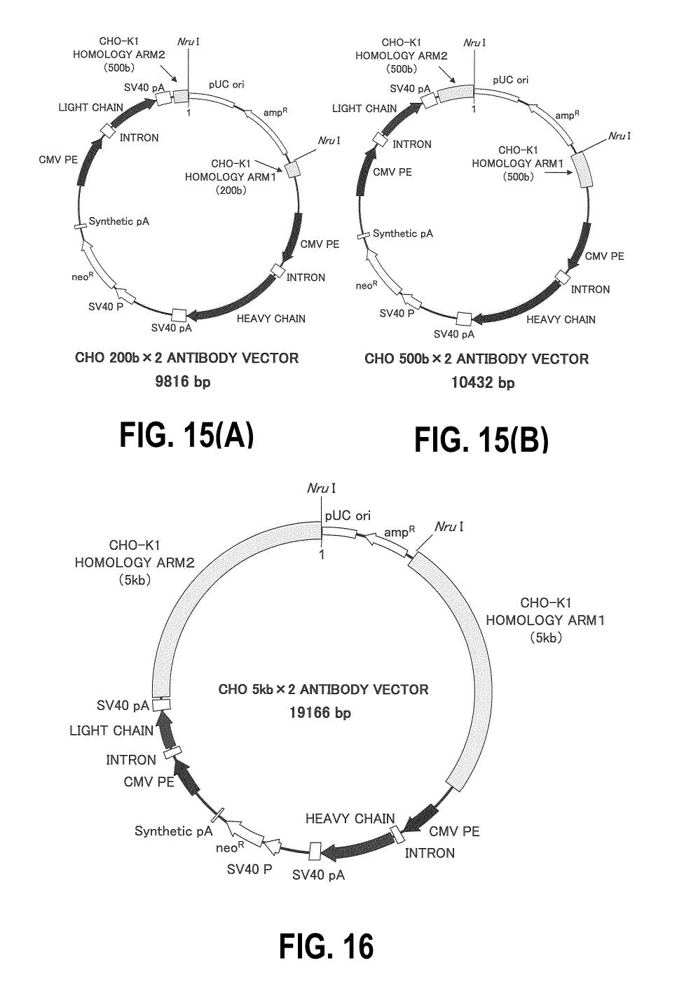

The basic vector+neoR+IgL plasmid of Example 2 was cleaved with restriction enzymes Bam HI and Pci I, followed by replacement with the CHA2-200 fragment. The product was then cleaved with restriction enzymes Sse8387I and Mlu I, followed by replacement with the CHA1-200 fragment. Finally, DNA sequence 8 (heavy chain cassette) was inserted into the restriction enzyme Mlu I site to obtain a CHO 200b×2 vector having a homology arm length of 200b shown in FIG. 15 (A).

On the other hand, homologous DNA fragments of a target hprt gene (CHA1-500 and CHA2-500) were cloned by a PCR reaction respectively with an SseNru-CHA1-500-s/MluI-CHA1-r primer set and a Bam-CHA2-s/PciNru-CHA2-500-r primer set.

SseNru-CHA1-500-s primer:
(SEQ ID No. 87)
5′-ttCCTGCAGGTCGCGAgatgcctagcatgtacctgg-3′
PciNru-CHA2-500-r primer:
(SEQ ID No. 88)
5′-ttACATGTTCGCGAtaagtacaaatccatcttgggtgac-3′

Next, the basic vector+neo R+IgL plasmid of Example 2 was cleaved with restriction enzymes Bam HI and Pci I, followed by replacement with the CHA2-500 fragment. Next, the product was cleaved with restriction enzymes Sse8387I and Mlu I, followed by replacement with the CHA1-500 fragment. Finally, DNA sequence 8 (heavy chain cassette) was inserted into the restriction enzyme Mlu I site to obtain a CHO 500b×2 vector having a homology arm length of 500b shown in FIG. 15 (B).

Acquisition of CHO 200b×2 Vector and CHO 500b×2 Vector Recombinant CHO Cells

Linearization, introduction into CHO cells, and screening of the constructed CHO 200b×2 vector and CHO 500b×2 vector were carried out in the same manner as in Example 3.

Despite the fact that the introduction operation was carried out twice, recombinant CHO cells were not acquired at all in any of the CHO 200b×2 vector and the CHO 500b×2 vector.

### Example 13

### Acquisition of Homologous Recombinant by Vector that has Homologous DNA Fragment Having Chain Length of 5 kb and Expresses Antibody Heavy Chain

Manufacturing of CHO 5 kb×2 Vector

Homologous DNA fragments of a target hprt gene (CHA1-5k and CHA2-5k) were cloned by a PCR reaction respectively with an NsiNru-CHA1-5k-s/MluI-CHA1-r primer set and a BclI-CHA2-s/BspNru-CHA2-5k-r primer set using genome DNA of CHO-K1 cells as a template. CHA1-5k and CHA2-5k were set to a sequence homologous to a region containing exon 3 of the hprt gene. The following primer sequences were used in the PCR reaction.

NsiNru-CHA1-5k-s primer:
(SEQ ID No. 89)
5′-ttATGCATTCGCGAAtctcaggtgataggagacataagac-3′
BclI-CHA2-s primer:
(SEQ ID No. 90)
5′-ttTGATCAgactgaagagctactgtgta-3′
BspNru-CHA2-5k-r primer:
(SEQ ID No. 91)
5′-ttTCATGAaTCGCGAAtcagcactcaggagtcagag-3′

Next, the basic vector+neo R+IgL plasmid of Example 2 was cleaved with restriction enzymes Bam HI and Pci I, followed by replacement with the CHA2-5k fragment. Next, the product was cleaved with restriction enzymes Sse8387I and Mlu I, followed by replacement with the CHA1-5k fragment. Finally, DNA sequence 8 (heavy chain cassette) was inserted into the restriction enzyme Mlu I site to obtain a CHO 5 kb×2 vector having a homology arm length of 5 kb shown in FIG. 16.

Acquisition of CHO 5 kb×2 Vector Recombinant CHO Cells

Linearization, introduction into CHO cells, and screening of the constructed CHO 5 kb×2 vector were carried out in the same manner as in Example 3.

As a result, recombinant CHO cells of 7 clones per 1×10⁶ cells were acquired. The frequency of acquisition with the vector having an homology arm length of 2.5 kb of Example 7 was 4 clones per 1×10⁶ cells, indicating that the homomogy arm length of 5 kb provided better results.

In the following Examples (14 to 18), embodiments of the present invention where the vector further comprises a selective marker gene that is a dihydrofolate reductase gene (dhfr) will be described.

### Example 14

### Construction of dhfr Deficient CHO hprt Recombinant Vector

Construction of dhfr Deficient CHO hprt Recombinant Basic Vector

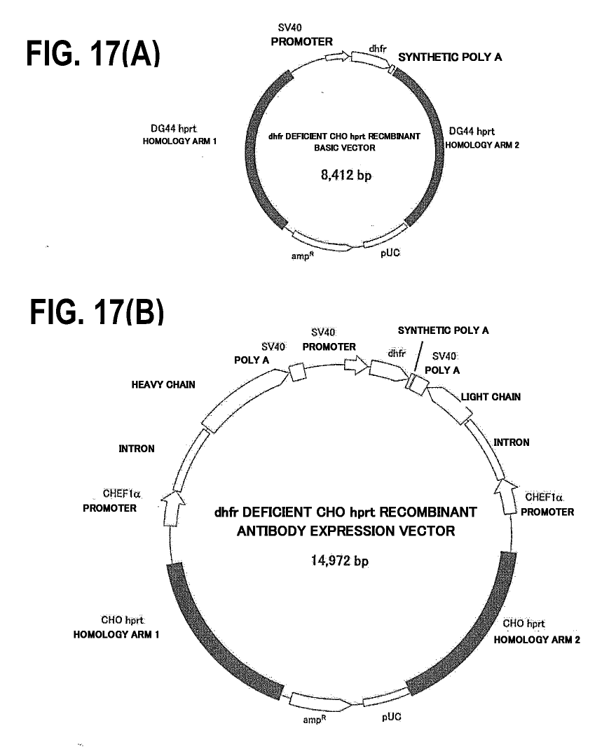

A vector (a dhfr deficient CHO hprt recombinant basic vector) shown in FIG. 17 (A) was constructed by the following method.

A DG44 cell line, a dhfr deficient Chinese hamster oocyte cell line, obtained from Invitrogen was cultured in a CO₂ incubator (37° C., 5% CO₂) using an AMEM medium (composition; Advanced MEM (GIBCO), 5% [v/v] FBS, 1× GlutaMAX (Invitrogen), 1×HT Supplement (Invitrogen)). The culture solution thus obtained was centrifuged to obtain DG44 cell pellets. The pellets were treated with DNA Isolation Kit for Cells and Tissues (Roche Diagnostics K.K.) to obtain genome DNA. Two DNA fragments of hprt locus of DG44 cells were obtained by PCR (KOD-Plus ver.2, TOYOBO) using the genome DNA as a template. Primers having the following sequences were used.

Homology arm 1 sense primer:
(SEQ ID No. 92)
5′-TCGCGAgtctgtgtgtatgtttgtgataggc-3′
Homology arm 1 antisense primer:
(SEQ ID No. 93)
5′-ACGCGTtgataaaatctacagtcatggg-3′
Homology arm 2 sense primer:
(SEQ ID No. 94)
5′-GGATCCgactgaagagctactgtgta-3′
Homology arm 2 antisense primer:
(SEQ ID No. 95)
5′-TCGCGAtgaaggttatagagcataggggacc-3′

In the PCR reaction, a recognition site of a restriction enzyme Nru I was added to the 5′ end of the homology arm 1 sense primer. Likewise, a recognition site of Mlu I was added to the 5′ end of the homology arm 1 antisense primer, a recognition site of Bam HI was added to the 5′ end of the homology arm 2 sense primer, and a recognition site of Nru I was added to the 5′ end of the homology arm 2 antisense primer.

A DNA sequence containing an on sequence, which is a replication origin within E. coli (Escherichia coli), and an ampicillin-resistant gene was cloned by a PCR reaction from DNA of a pQBI25 plasmid vector (Wako Pure Chemical Industries, Ltd.). The following primer sequences were used in the PCR reaction. In the PCR reaction, a recognition site of a restriction enzyme Nru I was added to the 5′ end of the sense primer and the antisense primer.

Ecoli sense primer:
(SEQ ID No. 96)
5′-TCGCGACCTGCAGGTCCGGAGCGGCCGCcatgtgagcaaaaggcc
a-3′
Ecoli antisense primer:
(SEQ ID No. 97)
5′-TCGCGACCTGCAGGTCCGGAGCGGCCGCaggtggcacttttcggg
g-3′

Next, a DNA linker obtained by annealing linker s oligo and linker a oligo was provided.

Linker s oligo:
(SEQ ID No. 98)
5′-CGCGTatcTCTAGAataATCGATagaAAGCTTacaG-3′
Linker a oliqo:
(SEQ ID No. 99)
5′-GATCCtgtAAGCTTtctATCGATtatTCTAGAgatA-3′

Homology arm 1, homology arm 2, and a DNA sequence containing an on sequence and an ampicillin-resistant gene were respectively cloned by a PCR reaction. These three DNA sequences were cleaved with restriction enzymes Nru I Mlu I and Bam HI, followed by a ligation reaction with the DNA linker to obtain a pHA12 plasmid vector.

Manufacturing of dhfr Expression Cassette

An SV40 promoter fragment was amplified by a PCR reaction from DNA of a pQBI15 plasmid vector (Wako Pure Chemical Industries, Ltd.). The following primer sequences were used for the PCR reaction. In the PCR reaction, a recognition site of a restriction enzyme Xba I was added to the 5′ end of the sense primer, and a recognition site of Pci I was added to the 5′ end of the antisense primer.

SV40 sense primer:
(SEQ ID No. 100)
5′-TCTAGActtctgaggcggaaagaacc-3′
SV40 antisense primer:
(SEQ ID No. 101)
5′-ACATGTcgaaacgatcctcatcctgt-3′

A dhfr fragment was then amplified by a PCR reaction from DNA of a pSV2-dhfr plasmid (ATCC, 37146). The following primer sequences were used for the PCR reaction. In the PCR reaction, a recognition site of a restriction enzyme Nco I was added to the 5′ end of the sense primer, and a poly A signal sequence and a recognition site of Cla I were added to the 5′ end of the antisense primer.

dhfr sense primer:
(SEQ ID No. 102)
5′-CCatggttcgaccattgaactg-3′
dhfr antisense primer:
(SEQ ID No. 103)
5′-ATCGATtCACACAAAAAACCAACACACAGATGTAATGAAAATAAAG
ATATTTTATTttagtctttcttctcgtagact-3′

The SV40 promoter fragment and the dhfr fragment that had been amplified by the PCR were cleaved with Pci I and Nco I, respectively, followed by a ligation reaction to ligate the cleaved fragments together to obtain a dhfr gene cassette.

Next, a pHA12 plasmid was cleaved with restriction enzymes Xba I and Cla I, and the dhfr gene cassette was inserted hereinto to obtain a dhfr deficient CHO hprt recombinant basic vector.

Construction of dhfr Deficient CHO hprt Recombinant Antibody Expression Vector

An antibody gene-containing vector (dhfr deficient CHO hprt recombinant antibody vector) shown in FIG. 17 (B) was constructed by the following method in order to integrate an antibody gene into an hprt locus.

Construction of Antibody Heavy Chain Expression Cassette

A variable region was amplified by PCR with a primer set of IGH-3A21s and IGH-3A21a1 using a plasmid holding a heavy chain variable region (“Cloning of cDNA and Characterization of Anti-RNase A Monoclonal Antibody 3A21”: Journal of Fermentation and Bioengineering. Vol. 82, No. 3, pp. 312-314, 1999) as a template to obtain a variable region fragment.

IGH-3A21s primer:
(SEQ ID No. 104)
5′-TAAAAGGTGTCCAGGATGTGCAGTTTCAGG-3′
IGH-3A21a1 primer:
(SEQ ID No. 105)
5′-GAGGCCGATGAAACAGTGACCAGAGTCCCT-3′

RNA extracted from RPMI8226 culture cells with ISOGEN (NIPPON GENE CO., LTD.) was used as a template, and RT-PCR was performed with one-step RT-PCR kit (QIAGEN). A constant region was amplified by PCR with a primer set of IGH-Cs and IGH-Ca using the resultant mRNA as a template to obtain a constant region fragment.

IGH-Cs primer:
(SEQ ID No. 106)
5′-CATCGGCCTCCACCAAGGGCCCATCGGTCT-3′
IGH-Ca primer:
(SEQ ID No. 107)
5′-TTAAGCGGCCGCTCATTTACCCGGAGACAG-3′

Next, PCR amplification was then performed with a primer set of IGH-LS and IGH-Va using the variable region fragment as a template to obtain a variable region fragment with a secretion signal added thereto.

IGH-LS primer:
(SEQ ID No. 108)
5′-GGTCGCCACCATGGAGTTTGGACTGAGCTGGGTTTTCCTTGTTGCT
ATTTTAAAAGGTGTCCAGGATGTG-3′
IGH-Va primer:
(SEQ ID No. 109)
5′-GCCCTTGGTGGAGGCCGATGAAACAGTGAC-3′

Assemble PCR was performed with a primer set of IGH-Vs and IGH-Ca using the variable region fragment with a secretion signal added thereto and the constant region fragment as a template to obtain an antibody heavy chain sequence comprising both the sequences linked to each other.

IGH-Vs primer:
(SEQ ID No. 110)
5′-TTCCGGTACCGGTCGCCACCATGGAGTTTG-3′

Next, a CHEF1αpromoter was amplified with an EF1aP-MluI-F/EF1aP-XbaI-R primer set using the DG44 genome DNA as a template.

EF1aP-MluI-F primer:
(SEQ ID No. 111)
5′-AGAACGCGTCCACACAATCAGAACCACA-3′
EF1aP-XbaI-R primer:
(SEQ ID No. 112)
5′-GACGATCTAGAGGTGGTTTTCACAACA-3′

The CHEF1αpromoter, the antibody heavy chain sequence, and the SV40 poly A were ligated together, and an Mlu I recognition sequence was added to both ends to obtain an antibody heavy chain expression cassette.

Construction of Antibody Light Chain Expression Cassette

Likewise, a plasmid holding a light chain variable region was provided as a template, and the variable region was amplified by PCR with a primer set of IGL-3A21s1 and IGL-3A21a1 to obtain a variable region fragment.

IGL-3A21s1 primer:
(SEQ ID No. 113)
5′-CCCAGGTGCCAGATGTGACATCAAGATGAC-3′
IGL-3A21a1 primer:
(SEQ ID No. 114)
5′-GCCACAGTTCGTTTTATTTCCAACTTTGTC-3′

RNA extracted from RPMI8226 culture cells with ISOGEN was provided as a template, and RT-PCR was performed with One-step RT-PCR kit. A constant region was amplified by PCR with a primer set of IGL-Cs and IGL-Ca using the resultant mRNA as a template to obtain a constant region fragment.

IGL-Cs primer:
(SEQ ID No. 115)
5′-TGGAAATAAAACGAACTGTGGCTGCACCAT-3′
IGL-Ca primer:
(SEQ ID No. 116)
5′-TTAAGCGGCCGCCTAACACTCTCCCCTGT-3′

PCR amplification was then performed with a primer set of IGL-LS and IGL-3A21a using DNA sequence 9 as a template to obtain a variable region fragment with a secretion signal added thereto.

IGL-LS primer:
(SEQ ID No. 117)
5′-GGTCGCCACCATGGACATGAGGGTCCCCGCTCAGCTCCTGGGGCTC
CTGCTGCTCTGGCTCCCAGGTGCCAGATGTGACA-3′
IGL-3A21a primer:
(SEQ ID No. 118)
5′-GCCACAGTTCGTTTTATTTCCAACTTTGTC-3′

Assemble PCR was performed with a primer set of IGL-Vs and IGL-Ca using the variable region fragment with a secretion signal added thereto and the constant region fragment as a template to obtain an antibody light chain sequence comprising both the products linked to each other.

IGL-Vs primer:
(SEQ ID No. 119)
5′-CCTTGGTACCGGTCGCCACCATGGACATGA-3′

The CHEF1α promoter, the antibody light chain sequence, and the SV40 poly A were ligated together, and an Hind III recognition sequence was added to both ends to obtain an antibody light chain expression cassette.

A dhfr deficient CHO hprt recombinant basic vector was cleaved with a restriction enzyme Mlu I, and an antibody heavy chain expression cassette was inserted thereinto. The product was cleaved with a restriction enzyme Hind III, and an antibody light chain cassette was inserted thereinto to obtain a dhfr deficient CHO hprt recombinant antibody expression vector.

### Example 15

### Introduction of Vector into Cells

Linearization of Vector

The dhfr deficient CHO hprt recombinant basic vector and dhfr deficient CHO hprt recombinant antibody expression vector were purified with Endofree Plasmid Maxi kit (QIAGEN) and were cleaved with Nru I, followed by dissolution in sterilized water to a concentration of 2 g/L, and the solutions were used in the following transfection experiment.

Transfection and Screening

DG44 cells were prepared with a nucleofection T solution (amaxa) to 1×10⁶ cells and were mixed with 2 μg of a linearized plasmid vector. Next, the mixed solution thus obtained and Nuceofector II (amaxa) were provided, and voltage was applied with program U-023. The transfection was performed twice for each vector. The transfected cells were seeded in a 96 well plate at 100 cells/well and cultured in an incubator under conditions of 37° C. and 5% CO₂ (medium: Advanced MEM (GIBCO) supplemented with 5% FBS, 1× Glutamax (Invitrogen), and 1×HT supplement (Invitrogen)).

After the transfection, the HT supplement was removed, and the medium was replaced by a medium supplemented with 6-TG (final concentration; 50 μM) (Wako Pure Chemical Industries, Ltd.), and the culture was continued. After the culture, all the wells were confirmed, and HT supplement non-requiring/6TG resistant colonies were isolated.

As a result, the number of HT supplement non-requiring/6TG resistant clones obtained by the dhfr deficient CHO hprt recombinant basic vector was 4 clones per 2×10⁶ cells.

### Example 16

### Analysis by Genome PCR

Genome DNA was extracted with DNA Isolation kit (Roche) from the HT supplement non-requiring and 6TG resistant clones obtained by the transfection of the dhfr deficient CHO hprt recombinant basic vector. Recombination regiospecific to the target hprt locus was confirmed by the following PCR reaction using the genome DNA as a template.

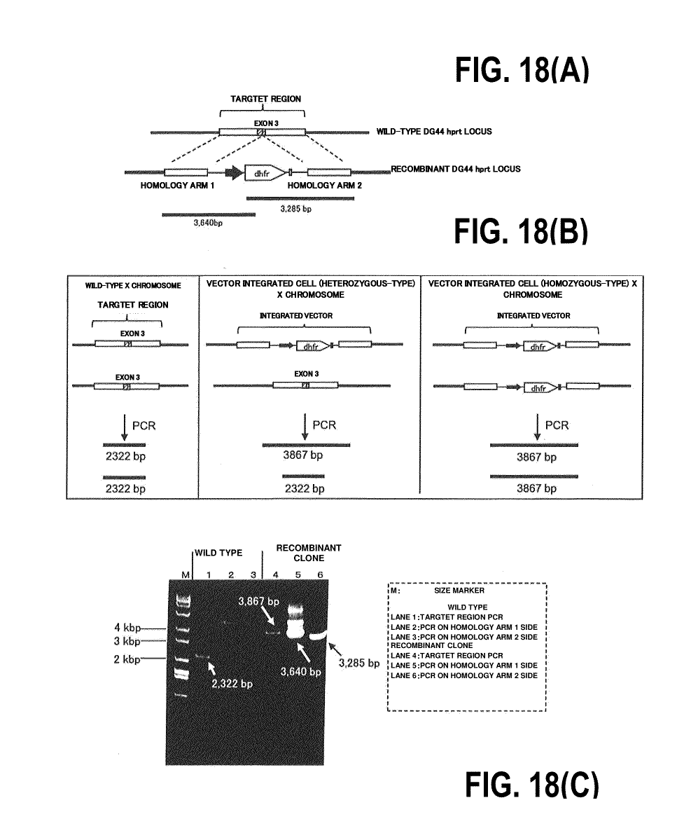

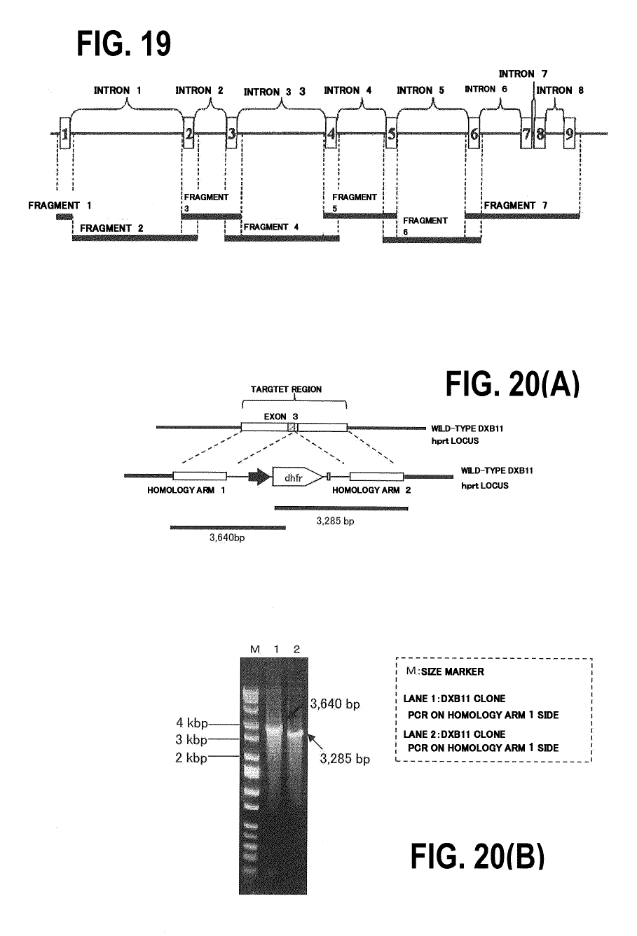

FIG. 18 (A) is a typical diagram for explaining details of an analysis of a homologous recombination reaction by genome PCR. A target region in homologous recombination of the hprt gene is set so as to contain exon 3. The homology arm 1, the homology arm 2, and the dhfr gene expression cassette held between the homology arm 1 and the homology arm 2 in the vector DNA are integrated into this region by homologous recombination.

DNA (3640-bp DNA) containing the homology arm 1 and a part of the dhfr gene expression cassette and DNA (3285-bp DNA) containing the homology arm 2 and a part of the dhfr gene expression cassette can be acquired from the genome only by homologous recombination and thus can be an index for homologous recombination.

Accordingly, a 3640-bp DNA shown in FIG. 18 (A) was set as an index for homologous recombination between the homology arm 1 and the target region, and the DNA was detected by a PCR reaction with primer CHA1seq-s11/dhfr-seq-a1 as shown below. On the other hand, 3285-bp DNA shown in FIG. 18 (A) was set as an index for homologous recombination between the homology arm and the target hprt locus, and the DNA was detected by a PCR reaction with primer NcoI-dhfr-s/CHA2-seq-a4 as shown below.

CHA1seq-s11 primer:
(SEQ ID No. 120)
5′-GACACATGCAGACAGAACAG-3′
CHA2-seq-a4 primer:
(SEQ ID No. 121)
5′-GTTTGCTAACACCCCTTCTC-3′
dhfr-seq-a1 primer:
(SEQ ID No. 122)
5′-ctcaggaatggagaaccagg-3′
NcoI-dhfr-s primer:
(SEQ ID No. 123)
5′-ttaaCCatggttcgaccattgaactg-3′

Next, the PCR amplification products thus obtained were analyzed by electrophoresis on 1.0% agarose gel. The results were as shown in FIG. 18 (C). In FIG. 18 (C), both the 3640-bp and 3285-bp PCR amplification products were confirmed only in the acquired clones, indicating that a homologous recombination reaction took place.

Whether or not a wild-type hprt gene remains was confirmed by the following PCR reaction for genome DNA of the HT supplement non-requiring and 6TG resistant clones obtained by the transfection of the dhfr deficient CHO hprt recombinant basic vector.

FIG. 18 (B) is a typical diagram for explaining details of genome PCR for the confirmation of remained wild-type hprt gene. At the outset, the left drawing in FIG. 18 (B) shows PCR in wild-type DG44 cells having two X chromosomes. A target region in homologous recombination of an hprt gene is set so as to contain exon 3. In vector non-introduced wild-type cells, DNA (2322 bp) of the target region can be amplified by a PCR reaction, and, thus, the remaining of the wild-type hprt gene can be confirmed using this as an index.

The drawing located at the center of FIG. 18 (B) shows PCR in heterozygous-type homologous recombinant cells comprising a vector integrated into an hprt locus of one X chromosome. In one chromosome, vector DNA is integrated into the target region by homologous recombination, and, thus, a 3867-bp PCR amplification product is produced. On the other hand, another chromosome holds a target region, and DNA (2322 bp) in the target region as well can be amplified by a PCR reaction, making it possible to confirm the remains of the wild-type hprt gene.

On the other hand, as shown in the drawing located right in FIG. 18 (B), in homozygous-type homologous recombinant cells comprising a vector integrated into both X chromosomes, only the recombinant DNA-derived 3867-bp PCR amplification product is expressed, and DNA (2322 bp) derived from the wild-type hprt gene is not amplified.

Based on the foregoing understanding, the 2322-bp DNA shown in FIG. 18 (B) was set as the index, and, for detection of the DNA, a PCR reaction was carried out with primer CHPRTs/CHA2-seq-a1.

Next, the PCR amplification product thus obtained was analyzed by electrophoresis on 1.0% agarose gel. The results were as shown in FIG. 18 (C).

CHPRTs primer:
(SEQ ID No. 124)
5′-TGTTCCTGTGCATACTAGGC-3′
CHA2-seq-a1 primer:
(SEQ ID No. 125)
5′-CCAGAGAAATTATTTGCCACCAGC-3′

In FIG. 18 (C), 2322-bp PCR amplification products were confirmed from the wild-type cells, and, thus, the presence of the wild-type hprt gene was confirmed. On the other hand, in the recombinant clones, any 2322-bp PCR amplification product was not confirmed, and only 3867-bp products were confirmed, indicating that the recombinant cells were of a homozygous type in which a vector was integrated into both X chromosomes.

From the results, it was confirmed that the use of the vector according to the present invention allowed recombinant cell clones comprising a target protein gene integrated into both hprt locuses of two X chromosomes to be acquired with high frequency.

### Example 17

### Manufacturing of DXB11 (dhfr Deficient CHO) hprt Recombinant Clone

Introduction of Vector into DXB11 and Screening

The dhfr deficient CHO hprt recombinant basic vector manufactured in Example 14 was purified and linearlized in the same manner as in Example 15 and was transfected into 3×10⁶ DXB11 cells, followed by screening.

As a result, one HT supplement non-requiring/6TG resistant DXB11 clone was acquired.

Confirmation of Integration of Vector into hprt Locus of DXB11 by Genome PCR

Genome DNA was extracted from the HT supplement non-requiring and 6TG resistant DXB11 clones obtained by the transfection of the dhfr deficient CHO hprt recombinant basic vector in the same manner as in Example 16. Recombination regiospecific to the target hprt locus was confirmed by the following PCR reaction using the genome DNA as a template.

FIG. 20 (A) is a typical diagram for explaining details of an analysis of a homologous recombination reaction by genome PCR.

In quite the same manner as in Example 16, a target region in homologous recombination of the hprt gene in DXB11 is set so as to contain exon 3. The homology arm 1, the homology arm 2, and the dhfr gene expression cassette held between the homology arm 1 and the homology arm 2 in the vector DNA are integrated into this region by homologous recombination.

DNA (3640-bp DNA) containing the homology arm 1 and a part of the dhfr gene expression cassette and DNA (3285-bp DNA) containing the homology arm 2 and a part of the dhfr gene expression cassette can be acquired from the genome only by homologous recombination and thus can be an index for homologous recombination. These DNAs were detected by a PCR reaction with the same primer set as that in the Example 16 using the genome DNA of the acquired DXB11 clone as a template.

The PCR amplification products thus obtained were analyzed by electrophoresis on 1.0% agarose gel. The results were as shown in FIG. 20 (B). Both the 3640-bp and 3285-bp PCR amplification products were confirmed in the acquired DXB11 clones, indicating that a homologous recombination reaction took place.

The above results demonstrate that a clone comprising a vector integrated into an hprt gene in DXB11 could be acquired.

### Example 18

### Manufacturing of dhfr Deficient CHO hprt Recombinant Antibody Expression Vector Clone and Antibody Production

Manufacturing of dhfr Deficient CHO hprt Recombinant Antibody expression clone

The dhfr deficient CHO hprt recombinant antibody expression vector manufactured in Example 14 was purified and linearlized in the same manner as in Example 15 and was transfected into 3×10⁶ DG44 cells, followed by screening.

As a result, one HT supplement non-requiring/6TG resistant clone was acquired by the dhfr deficient CHO hprt recombinant antibody expression vector.

Genome DNA was extracted from the acquired clone, and PCR was carried out in the same manner as in Example 15, except that the extracted genome DNA was used as a template. It was confirmed that the dhfr deficient CHO hprt recombinant antibody expression vector was integrated into the hprt locus.

Serum-Free Floating Habituation of dhfr Deficient CHO hprt Recombinant Antibody Expression Clone

The DG44 dhfr deficient CHO hprt recombinant antibody expression vector clone seeded in a 100-mm dish was cultured to 70% confluent and was then peeled off by trypsin treatment.

The cells were precipitated by centrifugation at 1000 rpm for 5 mins. The supernatant was discarded, and the residue was prepared to 3×10⁵ cells/ml with a complete CD OptiCHO medium (CD OptiCHO (Invitrogen) supplemented with 1× Glutamax (Invitrogen)). The prepared cells were transferred to a flask and were subjected to shaking culture in an incubator under conditions of 37° C. and 8% CO₂ at 121 rpm.

When the cell density exceeded 1×10⁶ cells/ml, the cells were diluted with complete CD OptiCHO to 3×10⁵ cells/ml for passage and the culture was then continued.

Habituation to serum-free floating culture was finished when the passage was repeated thrice.

Production of Antibody by dhfr Deficient CHO hprt Recombinant Antibody Expression Clone

The DG44 dhfr deficient CHO hprt recombinant antibody expression vector clone habituated to serum-free floating culture was pelletized by centrifugation at 1000 rpm for 5 mins, and the pellets were suspended in a fresh complete CD OptiCHO medium. The cell suspension was diluted with a complete CD OptiCHO medium to prepare 60 ml of a cell suspension of 3×10⁵ cells/ml, and the cell suspension was transferred to a flask.

The cells were subjected to shaking culture in an incubator under conditions of 37° C. and 8% CO₂ at 121 rpm. The cell density was measured every 24 hrs and was recorded. Further, 1.5 ml of the medium was collected and was centrifuged, and the supernatant was stored at −80° C.

Sampling was carried out for 18 days, and the amount of IgG in the collected medium and the sampled medium was determined by measuring absorbance at 450 nm with Human IgG EIA Kit (precoated) (manufactured by Takara Shuzo Co., Ltd.).

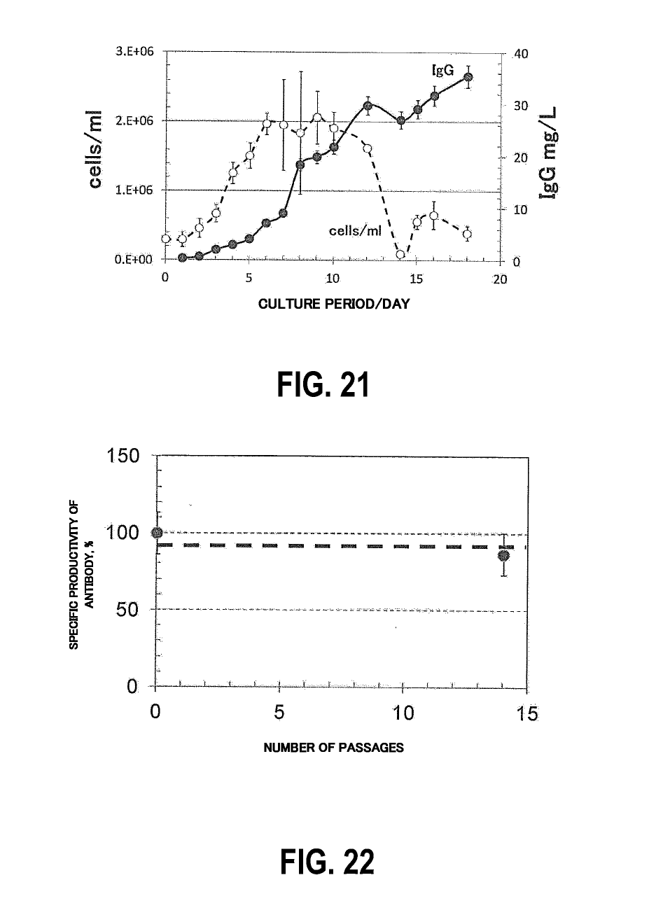

As a result, as shown in FIG. 21, it was found that the production of IgG was 35 mg per L of the supernatant of the medium.

Confirmation of Long-Term Stability of Expression of dhfr Deficient CHO hprt Recombinant Antibody Expression Clone

The DG44 dhfr deficient CHO hprt recombinant antibody expression vector clone habituated to serum-free floating culture was subcultured 14 times for maintenance. The passage was carried out twice per week. Thereafter, a part of the cell population was prepared to 3×10⁵ cells/ml with 60 ml of a fresh complete CD OptiCHO medium. The prepared cells were transferred to a flask, followed by start of culture.

On the 7th day from the start of culture, the density of cells in the medium was measured. The supernatant (1.5 ml) of the medium was collected and was centrifuged, and the supernatant was stored at −80° C. After the elapse of 24 hrs from the start of the storage, the cell density was measured, and 1.5 ml of the supernatant of the medium was stored in the same manner as described just above.

The stored samples of the supernatant of the medium were lysed and were subjected to the determination of the amount of IgG with Human IgG EIA Kit, and the specific productivity (pg/cell/day) was calculated based on the cell density. Further, the DG44 dhfr deficient CHO hprt recombinant antibody expression vector clone just after habituation to serum-free floating culture was also subjected to the determination of the specific productivity in the same manner as described just above.

As can be seen from FIG. 22, after 14 passages, the specific productivity of clone was not lowered. This can be said to be favorable because the maintenance of stability of 50 days (about 7 weeks) that is a minimal stability requirement for actual production is satisfied.

Some preferred embodiments of the present invention are as follows.

(1) A DNA construct comprising, from a 5′ end toward a 3′ end, a first homologous DNA fragment, a target protein gene, a selective marker gene, and a second homologous DNA fragment,

the first and second homologous DNA fragments having homology allowing for homologous recombination with a part of a hypoxanthine-phosphoribosyltransferase enzyme (hprt) locus in a dhfr gene deficient CHO cell genome and having a chain length of not less than 1 kbp, the selective marker gene being a dihydrofolate reductase gene (dhfr).

(2) The DNA construct according to (1), wherein the first and second homologous DNA fragments have a chain length of 1 kbp to 7.5 kbp.

(3) The DNA construct according to (1), wherein the first and second homologous DNA fragments have a chain length of 1 kbp to 5 kbp.

(4) The DNA construct according to any of (1) to (3), wherein the part of the hprt locus is a region including at least a part of introns of the hprt gene.

(5) The DNA construct according to any of (1) to (4), wherein the first or second homologous DNA fragment includes a base sequence described in any of SEQ ID Nos. 35 to 42 or a partial sequence thereof.

(6) The DNA construct according to any of (1) to (5), which comprises a CHEF-1α gene promoter operatively linked to the target protein gene.

(7) A vector comprising the DNA construct according to any of (1) to

(6).

(8) The vector according to (7), wherein the vector is a plasmid vector, a cosmid vector, a phage vector, or an artificial chromosome vector.

(9) A process for generating a recombinant CHO cell, the process comprising: introducing the vector according to (8) into a dhfr gene deficient CHO cells; and screening cells with the vector integrated into the hprt locus using hypoxanthine and thymidine non-requirement and 6-thioguanine (6tg) resistance as an index.

(10) A recombinant CHO cell generated by a process according to (9).

(11) A process for producing a target protein, the process comprising: providing the recombinant CHO cell according to (10); and culturing the cell to produce a target protein.

1. A DNA construct comprising, from a 5′ end toward a 3′ end, a first homologous DNA fragment, a target protein gene, and a second homologous DNA fragment,
wherein the first and second homologous DNA fragments have homology allowing for homologous recombination with a part of a hypoxanthine-phosphoribosyltransferase enzyme (hprt) locus in a CHO cell genome and having a chain length of not less than 1 kbp.

2. The DNA construct according to claim 1, wherein the first and second homologous DNA fragments have a chain length of 1 kbp to 7.5 kbp.

3. The DNA construct according to claim 1, wherein the first and second homologous DNA fragments have a chain length of 1 kbp to 5 kbp.

4. The DNA construct according to claim 1, wherein the part of the hprt locus is a region which includes at least a part of introns of the hprt gene.

5. The DNA construct according to claim 1, wherein the first or second homologous DNA fragment includes a base sequence described in any one of SEQ ID Nos 15 to 22 or a partial sequence thereof.

6. The DNA construct according to claim 1, wherein the target protein is an antibody, an enzyme, a cytokine, a hormone, a coagulation factor, a regulatory protein, or a receptor.

7. The DNA construct according to claim 1, which further comprises a positive selective marker gene.

8. The DNA construct according to claim 7, wherein the positive selective marker gene is a dihydrofolate reductase gene (dhfr).

9. The DNA construct according to claim 1, wherein the CHO cells are dhfr gene deficient CHO cells.

10. A vector comprising the DNA construct according to claim 1.

11. The vector according to claim 10, wherein a nuclear/matrix adhesion region (MAR) derived from a first intron of a human hprt gene is maintained in such a structure that, after the integration of the vector into a chromosome, the MAR per se is transcribed by transcription at the integration site.

12. The vector according to claim 10, which comprises a CHEF-1α gene promoter operatively linked to the target protein gene.

13. The vector according to claim 10, wherein the vector is a plasmid vector, a cosmid vector, a phage vector, or an artificial chromosome vector.

14. A process for generating a recombinant CHO cell, the process comprising introducing the vector according to claim 10 into a CHO cell.

15. A process for generating a recombinant CHO cell, the process comprising: introducing the vector according to claim 10 into dhfr gene deficient CHO cells; and selecting cells with the vector integrated into the hprt locus using hypoxanthine and thymidine non-requirement and 6-thioguanine (6TG) resistance as an index.

16. A recombinant CHO cell comprising an exogenous target protein gene integrated into an hprt locus.

17. The recombinant CHO cell according to claim 16, wherein the function of the hprt gene has been inactivated.

18. A recombinant CHO cells generated by a process according to claim 14.

19. A recombinant CHO cell generated by a process according to claim 15.

20. A process for producing a target protein, the process comprising: providing the recombinant CHO cell according to claim 16; and culturing the cell to produce a target protein.

21. A process for producing a target protein, the process comprising: providing the recombinant CHO cell according to claim 18; and culturing the cell to produce a target protein.
